SEQUENCE LISTING (21 sequences)

SEQ ID NO: 1 - DNA, 1867 bp, Aspergillus nidulans
  source 1..1867: mol_type = genomic DNA
atggccggcgagctcgatgagccaccagcggatatccagacgatgctggatgactttgatgtacaggtcgccgcgctgaagcaggatgccacggcaaccaccacaatgtcgacgtcgacagctctcatgcctgccccagccatctcatctaaagatgctgctcctgctggtgctggtttatcgtggcctgacccaacctggctggatcgccagtggcaggatgtcagcagtaccagcctcgtccctccatcagacctgacagtctcgtcggccactaccctaaccgaccctctcagcttcgaccttttgaacgagactcctcctcctccttctacgacgacaacaacgtcgacgacgaggcgagactcatgtactaaggtcatgttaactgacctcatccgggctgaattgtacactacctaactgatttgtctaccatgacacctgactgacaatgtgcagagaccaactctacttcgaccgggtccacgccttctgccccatcatccaccggcgacggtactttgcgcgggtcgcccgagatagccataccccagcacaggcatgtctgcagttcgccatgcgaacgctcgcagcggcaatgtctgctcactgccatcttagcgagcatctctatgccgagaccaaggccctcttggagacgcacagccagacgcccgccacaccgcgagacaaggtcccgctcgagcacatccaggcctggctgttgttaagccactacgagctgctgcggatcggcgtgcaccaggctatgctcacggctggccgggcctttcgtctcgtgcagatggcacgactgtcagagctggatgccgggtcagatcgacagctctcgccgccgtcttcgtcgccgccgtcttcgctaaccctatctccttcgggggagaatgctgagaacttcgtcgacgccgaagaaggccggcggacgttctggcttgcttattgctttgatcgtttgctttgcttgcagaatgagtggccgttaacgttacaagaagagatggtacgtcgcgcttcttttattctatttacctcagaatttatattcagttattttttattctaaccctgctagatattaacccgcctcccctccctcgaacacaactaccagaacaatctccccgcacgcacgccctttctcactgaagccatggcccagaccgggcagagcacaatgtccccgtttgccgaatgcattatcatggccacccttcacggccgatgtatgacgcaccgccgcttctacgcaaacagcaactcgactgcgtccggctccgagttcgagtctggcgccgcgacgcgagacttctgtatccgccagaattggctgtcgaatgcagtggaccggcgagtccagatgctacagcaggtctcctcgcccgctgttgacagcgacccgatgctgctcttcacgcagacgctcggctaccgcgcgaccatgcacctgagcgataccgtccagcaagtctcctggcgggctctcgccagctcgcccgttgaccagcagctactgagcccgggcgcgacgatgtcgctgtcggccgccgcgtaccaccagatggccagccacgcagccggcgagatcgtccgcctggcgaaggccgtcccctcgctgagtccgttcaaggcgcacccgttcctacccgatacgttggcgtgcgccgccacgttcctctcgacgggcagtcccgatcccacgggcggcgagggggtgcagcatctgctacgagtgttaagcgagctgcgcgatacacacagcctggcgcgggattatttgcaggggttgtcggtgcagacgcaggacgaagatcatagacaggatacgaggtggtattgtacatag

SEQ ID NO: 2 - DNA, 1452 bp, Aspergillus nidulans
  source 1..1452: mol_type = genomic DNA
atggcagaccacgagcaggaacaggaacccctctccatcgccatcataggcggcggcatcatcggccttatgacagccctggggcttctccaccgcaacatcggcaaggtgacaatctacgagcgcgcctcagcttggcccgacattggggccgcatttgccttcaccggaatcgcccgcgagtgcatgcagcgccttgatcccgcaattctatccgctctctccaaagttgcgcagcgcaatccgcatgataaagtgcggtactgggacggattccacccgaaaagcaaagaggaggcacaggatccagagaagagcgtgctgtttgagatcgaggagaagaatatggcgtactgggcttgcttgaggggtgtttttcatgcggagatggcgcggttgctgcccgagagggttgtgaggtttggcaagaggctggtggcatatgaggatggtggggaccagaaagtggtgctgaggttcgaggacggggaggtggaggaggcagatattggttggttttcttccctgagctgggtattggacgtttcataatgctaatgcggtgggtgaagtgatcgcatgcgatggcgtccactccacggcccgcagagtcctcctcggcgcagaacaccctgctgcaaacgccaggtactctcgcaaagctgtctaccgcgctctcgtgcctatgccagcagcaattgatgcgctggggaccgaaaaggcgcatgtccagatcgcacattgcgggccggacgcgcacattgtctcttttccggtaccctaccttacctccaggcctagcatgccgcatctattctgcaagaaggcgctgattgtgcctaggtaaacaacgcccagatctataatgttttcctcttcacgcacgactcgaacgagtggactcacggccacaccatgacagtgcccagctcgaaggaggagatactctcggcggtcgagaactggggcccgcatatcaaggaacttgccagcctcttcccggaacagctaagcaagtacgccatcttcgatcaggcggaccacccgcttccttactatgccgctggccgggtggcgctggccggtgatgccgcgcacgcgtccagtccgttccacggggctggggcctgtatgggcgttgaggacgcgcttgttctcgctgagttgcttgagaaggtccagaacggctccgcgttcaaggagaagaagtctaatattgagcttgccctgaagacttacagcgatgtgaggattgagcgtagccagtggcttgtgaagagctcaagggagatgggagatctctacgaatggcggtatgaagacattgggggtgacggggtaaagtgtaaggctgaatgggagaggaggagcagggtgatttgggatttcgatgtgcagggcatggtcgaccaggcgagagaagcgtatgagcgtgctgtagtgaaggtctag

SEQ ID NO: 3 - DNA, 2526 bp, Aspergillus nidulans
  source 1..2526: mol_type = genomic DNA
atggcagatacgcgccgacgccagaatcatagctgcgatccctgtcgcaagggcaagcgacgctgtgatgccccggtaggttgccgatatcggctccccagcgtgtgcactgacagtcgctgagatgtaacacaggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttgtag

SEQ ID NO: 4 - DNA, 7527 bp, Aspergillus terreus
  source 1..7527: mol_type = genomic DNA
atggcacccatggagccgattgccatcgttggcactgcctgccgatttgccggctcgtcatccactccttccaggctttgggaacttctcttaaaccccaaggacgtggcatcagagccacccgcagatcgattcaatatcgatgctttctatgacccggaaggctccaaccccatggcgaccaatgcccgccaggggtatttcctttctgacaacgtcaaagccttcgatgccccgttcttcaatatctccgcagccgaagcactggcactcgacccacagcagcggatgctgctggaagtcgtctatgaatcactggagactgctggcctgcgcttagacactctccgcggctcctcgacgggggtctactgcggtgtgatgaactccgactgggagggcatattcagcgtctcatgtgcagcaccgcagtatgggagtgttggggttgcccggaataacctcgctaaccgcatctcctacttcttcgactggcaaggcccgtccatgtccatcgataccgcctgctcagcgagcatggtagcattgcatgatgccgtctccgcactcactcgccacgactgcgacatggctgcagctctaggtgccaacctcatgttgtctccccagatgttcatcgctgcatccaatttgcagatgttgtccccaaccagccgcagccgtatgtgggatgcgcaggctgatggttatgcgcgtggcgagggggtcgcatccgtgctcttgaaacggctttcagatgcagtggccgacggcgaccctatcgaatgtgttatccgagctgtcggcgtgaaccatgatggccgtagcatgggtttcaccatgccgtcgagtgatgcacaagtgcaactgatcaggtctacttatgcaaaagccggattggatcctcgctgcgcggaagatcgaccccaatatgtcgaggcccatggtacaggcacgttggcgggtgatccccaggaagcatccgcccttcatcaggccttcttcagttcctcggacgaggacactgtactgcatgtcggttccatcaagacagtggtaggccacgcggaagggactgctggtctcgcgggtctcatcaaggcatccctgtgcattcagcatggcataatacccccgaatcttcttttcaatcgcttgaacccggctctggagccatatgcacggcaattgcgagttccagtagacgtgatcccctggccctcccttcctccaggcgttccccgacgtgtttcagtgaactccttcggctttggtggcaccaatgctcatgttattctggagagctatgaacctgctagagacctcaccaaggacggcttcaatcagaatgcggtgcttccgtttgtcttctctgcggagtcggattatagtcttgggtcggttctggagcagtattccagatatctctccagattttctgacgtggacgtacacgatctggcatggacgctaatcgagcgccgttccgcgctgatgcaccgtgtcgctttttgggcgccagatattgcacacctcaaaagaaggatccaggatgaggtcgccctccggaaagcagggacaccctcgacagtcatctgccggccacatggcaagactaggaagcacattctgggcgtcttcactggtcagggtgcccaatgggcgcagatgggacttgaactaatcaccgcgtccaccattgcgcgaggctggctggatgagctgcaacagtctctcgatactttgccggaggcgtatcgtccagagttctcgctctttcaagagcttgctgcggatccggccgcatcacgactatcggaggcccttctgtcgcagaccctctgcacagcaatgcagattatctgggtgaaggtgctctgggctctgaacatccacttggaagctgtggtcggtcactcatctggcgagattgctgcggcctttgcggctggctttctgacagctgaggatgccattcgcattgcctaccttcgaggtgtgttttgctcggcttcaggcagctcgggggaaggtgcgatgctggccgctggtctttcgatggacgaagcgactgcactctgtgacgacgtatcctcgtctggggggcgaatcaacgtggcagcgtccaactcgcctgaaagcgtcacgctctctggagaccgagatgcaattctgcgagctgagcagcagttgaaggataggggagtctttgcccgtctacttcgtgtcagtaccgcctaccactcccatcacatgcagccatgttcgcagccctatcagaacgcattgagtagttgcaacattcagattcaggccccggtgcccaccaccacctggtattcaagcgtctatgctgggtgccccctggaggagccttcggtcatagagacgctcggtacaggagaatactgggcggaaaatctagtcagtcctgtgttgttctcgcaggcactaacggctgccatatccaccacaaacccttccctggtcgtcgaagttggacctcatccagctctgaaaggacctgccttacagacgatctcaggaataacgtcaggggagatcccttatatcggggtatcagcccggaacaattgtgcacttgagtccatagccacagccattggatctttctggacgcatcttggtccacaagtcatcaatccgcgagggtacctggctcttttccggccgaatgtgaggtcttcagttgtccgtgggctgcctttgtatccctttgaccatcgccaagagcacggttatcagacccgcaaggctaatggttggctgtaccgacggtacacaccacaccctctgctgggttctctgagtgaagacctcggggagggcgagttgcggtggaatcattacctctccccccgacggctcccatggctcgatggccaccgcgtccagggccaaatcgtggtccctgccacagcttatatcgtgatggctctcgaggccgctcgcatactgaccgctgagaaacaaaagagcttgcatctaatccgtatagacgacctagtcatcggtcaagctatctccttccaggatgaacgagatgaggttgagactctgttccacctcgcccctatggtggagaccaaggatgacaacacagcagtcggccggttccgctgtcagatggctgcttccgggggtcacgtcaagacatgtgcggagggcatcctcacggtaacctggggctcgccgctggatgatgtcctcccataccctaggtctccagcgcccgcagggctagcccatgtagccgacatagacgagtactatgcgtcgctccgaagcttgggttacgagtacaccggcgccttccagggaattttttctctctcccggaagatgggtatcgccacgggccaattgtgtaaccctgcattaaatggctttctgatccatccagcagttctcgacactggattacagggtcttctggccgcggtgggggagggacacctcacgagcctacatgttccaacccgcattgatgcattcagcgtaaaccctgcagcctgtagtagcggttcgctagcctttgaggctgccgtgactcggacaggattagacggtctcgtgggcgacgtggagttgtatacggataccaacggccctggtgccgtcttctttgaaggagtgcacgtctccccactagtgccgccatccgcagcggatgatccgtcagtattttgggtgcagcattggacaccccttagcctggatgtcaaccgttccaaatctcgactgtcgccggaatggatggccatgttagaagggtatgagcgccgggcgttccttgcactgaaggacatcctccagcaggtcacaccagagcttcgtgccacttttgactggcatcgtgaaagcgttgtcagttggattgagcacattatggaggaaacccgcgtgggtcggcacgccgtctgcaagcctgagtggctagaccaagagctagagaatctcggacacatatgggggcggccagacgcgcgcattgaggatcgaatgatgtatcgagtttaccggaacctgctacccttcctccgcggggaagcgaagatgctagatgctcttcggcaggacgaattgcttacacagttctatcgcgacgagcacgagctgcgcgatatcaaccgtcgactgggtcagttggttggtgacctagccgtgcgctttccacgtatgaaactccttgaagtcggcgccgggacaggctctgccactcgagaggtactcaaacatgtcggccgggcctaccattcctacacgttcacagacatctcggttggcttttttgaagacatgttggaaacaattcccgagcacgcggaccgtctgctattccagaagctcgatgtcgggcaagacccattgcagcagggctttggtgaacacacttacgatgtaatcatcgccgctaacgtacttcatgccacaccgacgctgcaagagactctgcgaaacgtgcgtcgtctactcaagccaggagggtatctgatcgctctggagatcactaacattgatacaatccgcatcggcttcttgatgtgtgcctttgacggctggtggcttggccgggaggatggccgtccatggggtccggtggtctctgcatcacagtgggatagcctactccgggagacgggattcggtggcatagacactatcactgatcgcgccgctgaccagctcaccatgtactctgtctttgccgcccaagcggtggacgaccagatcactcgatgtcgagaacctctgacgccgctccctcctcaacctcctttctgccggggagtgatcatcggaggctcgcctagtctggtgacaggcataagagtcattattcatcctttcttctcgactgttgaacatgtttctaccatcgagaacctgacggagggagcaccagctgttgtgttgatgttggctgacctgagcgacatcccctgcttcgaaaatctcaccgagtcaagactggccggactcaaagcactggtgcaaatggccgagaagacgctctgggtgaccacgggctctgaagcggacaacccttatctctgcctcagcaagggctttctcacttcgatgaattatgaacatccagctatcttccaatatctgaacatcatcgactcggctgacgtccaacccgtggtcttggccgagcatcttctgcgattggcctataccaaccaaaacaatgacttcgccctcacgaattgcgtccacagcacagagcttgagctgcgtctctaccagggcgggattctgaagttcccacgcattaacgcgagcgatgtcctgaacagtcggtacgcggcagctcggcgcccagtcacccattctgtcaccaacatgcaggacagcgtggttgtacttgaccaaagcccaagtgggaagcttcgactcgtgtttggggaggagcttgcaggtgatcgcgcaaccgtcaccattaacgtccgatactcgacctctcgtgcaatccgcatcaatggtgctggatatctggtccttgttctcgggcaggataaagttaccaaagcgcgtctggtggctctggcaggtcagtctgcgagcgtcgtctcgtcctcctgttattgggaggtcccagcagatatcttcgaggagcaggagcccgcgtatctgtacgccacagcaacagctttgctcgctgccagtttggtgcagtccaacggcaccacaatcctggtacatggcgctgacatggtcctacgccatgcaatcgccatagaggccgcttcacgggtcattcagcctatattcactaccacatctccctccgcagcatcatccgcgggtcttgggaagagcatcctcgtgcatgagaacgacacccggcgacaactggttcatcttctccctcgatatttcacagctgctgtgaatttcgaccctagtgcccgccgactcttcgaccgaatgatgacagtcggtcatcaatcgggtgtcacagaagaacaccttcttaccactttgacagctgccctccctcgtccgtcagcatctctgctgccggcccagcctcaggctgccatggacactcttcgcaaagcctcattgactgcttatcagttcaccgtccagttgacagcaccaggacccatcatcgcaccaatcgccgacatccaatcctgttcacaacagttagcagtcgtagactggaaaccatcttgcggctcggttccagtacacctccaaccagccactgagctggttcgtctctctgctcaaaagacatatctcctggtgggtatgactggtgccctcggccaatccatcacgcaatggctggtcacccgcggcgctcgcaatatcgtcctcaccagccgcaagccatcagtggaccccgcatggatcgcagagatgcagaccacaacaagcgcgcgtgtcctcgttacgccaatggatgtgacaagccgcgactcgatccttgtggtggcacacgccctgaaggccgactggccgccgctcggcggcgtcgtcaacggtgccatggtgctctgggaccgtctcttcgtcgacgcacccctgtccgttctgacgggacagctcgccccaaaagtccaggggagccttctcctcgatgagatttttggccatgaaccgggccttgatttctttatcctcttcggtagcgctatcgccactattggaaatctgggtcagtctgcctacacagccgccagtaacttcatggtcgcgcttgcggcgcaacgccgcgcccgagggcttgtcgcaagcgtcctccagccggcgcaggtcgccggtgccatgggttatctcagggataaagacgacagcttctgggctcggatgtttgatatgattgggcgacatctcgtctccgaaccagatctgcacgaacttttggcccatgctatcttgtcgggtcgtggccctccagctgacgttggatacggaccaggcgaggatgagtgcatcattggcggactccgcgtccaagaccctgctgtatacccagatatcctctggttccgtacgcccaaagtctggccattcatccactatcaccacgagggaactggcccttcatctggggcggctggttcgatatcgctggtcgatcagctgaagtgtgcgactagcttagcccaagttggggacatggtggaagctggcgttgcggccaaactgcaccatcgactccatctcccaggcgaggttggaggcgtcactggcgacacgcgtttgaccgagctgggggtggactcgttaattgcggtggacttgcgtcggtggtttgcgcaggagttggaggttgatattcccgttctgcagatgctgagtgggtgttcagtaaaggagctggctgcttccgcgacggcgttgttgcatccgaaattctatccggaggtggtggccgattctgacgtggggagtgagagggatggttcctcggactcccgtggtgatacctcttcctcctcgtatcagctgatcactccggaggagggggaccatgactga

SEQ ID NO: 5 - DNA, 687 bp, Aspergillus terreus
  source 1..687: mol_type = genomic DNA
atgacctcctaccagctttccgatgccgaaggcgccgacnactactacaatccacttctcttgtggtggtacgacttctgggttcattgggtcagcgcgctctttgcctggaagtgttcgtctaaggacattcttctcccttttttcctgtccaacattggatctcggcactgtgatgttggcgtgggcacgggctactatctctctgctgtgcggaagcgtcggccgtcctggccggagaagctgacgctagtggattttcacatccgatgcctgaggaaggctgccaaccgcgttggagtagcggatcgcacggagtgtgtgctggccaatatcctggagcccatccctatacaacctgaacggcagttcgactccatatcccttatgtacgtcctgcactgtctcccggggacttctaaggacaaaggacgcgtgtttgccaacttgaagccgctcctcaaggacaatggcactctcttcgggtctacgctgctgtgccgtggagttcggcagaattggttcagttggctccttcagcgcatctacaatgctgtggacatgtttcagaatcggtcagactatccagacgactttgtccgtgcgctggaggacgagttcgaggaggtggagagcgtgattattggaacggtccttatgttcaaggcgcggaagcctcgtcgctag

SEQ ID NO: 6 - DNA, 1611 bp, Aspergillus terreus
  source 1..1611: mol_type = genomic DNA
atggaaggaaagcaccctcagttcaaggtcgtcatcgtcggggcatccgtcacgggtctgaccctcgcccactgtctgcatcgggccggaatagactacgtggtgctggagaagcatcacgaagtccatccgccgattggcgccgcggtggccatcctgccgaatggagggcggatcatggagcagctggggatcttccggcacattgaagaccgctgccagccatttcaaagggtgcatctttgctttcaggatgggttctactatgatagcctttcgcctagtgtcgtcttgaagaggtcggtgcttccttggtccccgagagggtggaacaaagtcgtaagaagacgctaacacggcgggtacgcaggtttggcttgaaattcgcgtgcctggaacggactcagctactcgagatcctgtacgcccatttgcctgacaagtccagggtcctcacgagcaaaggcgtcgtccggatcactccccatggcagcaagatgacggtcaccaccgcagatggggacgagttccagggagatctggttgtcggtgcggacggcgtgcacagtgtgacgcggcgagagatgtggcgcattgctaatatagagcagccggggctgattccgctcaaagaacaggccagtatgttgctacactccttatcgattccgtccaatattaatagatgatgctctgcggctaggcatgtccgtcgagttcagctgcgtcttcggcatgtccaaccccatccccggacggaagcgttggcagcatgtgatccgcatcgggcccggtttcacgatcctgatattccctgcgaccggggagagccttttctgggtgctgattgaaaagctaccacacaagtatatctaccctgacgttccgcgattctcgcaggaggacgccattgcgcgctgtgaggcagccgccagccagcctatctgggaggaagtacaattccgggatatctgggctcaacgccggggattccggatggttgcgttggaggagaatctcttccgtacgtggcatcatggccggatcatctgcatcggggatagtatcagcaaggtatgtcttgcatggagcctttttttccccttcatatactgatagctgggactctagatgacccccaacatcggtcaaggcgccaatacagccattgaagcggcggctggactggccaacgtgatatatgcgattgcacaaaaccatgaccagccgtcaagtgacaccattcaccaggcgctcgctgccttcagcgagaaaaaccgcaaacgactggatgctatccatctagagtcgcgctggatcacgcgattggaggcttgccagggacggatggtcacggccttcacgcgatatgtggcgccacactgtggcgatctttttgcgttgggggtggtgcgtaactcatacaacggtgaggtactgcagttcttgccgttgactgaacgttcggggaagtactggccgaagttggagtggtggaatacttggggtctgtccaagtggcaggaatttggggagagggtgatgtattgctctggacttgtgatagttttgtggattagctgggtggtgttcaatgtcaaggaagtatga

SEQ ID NO: 7 - DNA, 1132 bp, Aspergillus terreus
  source 1..1132: mol_type = genomic DNA
atggccctttcagcctacatacttctctgcttgtccgtacttggactagacgccatctatggcttcngattccggaatggcttcctcgagctcatggccaacagctaccgagagcgaaagctatccggcacggcagagcccttgcaaggcaacatcaccggaacgggatttgacgaactcttgggaaatctaattgtcttctactggcccgtgctggacggacgccatcctggtctcagtgtccaggcctttcacttctctggtgcaatcgtggccgtgtgggtggcgatccaggtccagagctggagatcgccaacccgcaccagcgttctgctatcgtgagtgtctctaccctcgtatccctattcttcatctgtcggccatgttccgaaccaaccgtcatagtccaacccttttcgccatgctatcccaggtcgtggccatcgccgtcgtcgtacccctctggtgcgcgatccacatctggtcgtcgtcgtcgtcgtcgtcatcatcatcatcacgcccagtggcgcgcgttatctcggcatcagcagctcattccatacgcctcatccccatcagcatgatcctgggatttggagtcccgactatcgccatggtccttccggaatccgcgcaccaggacttgttcggcaaacagatggccattgccgcatggcagctctggccaatctatgcagctctgtgtcactggggtctacgggcctttttccgaccaagagcgagcacgggtatctccacgaggagtgcatgccggaccgcgtatacctttgcgtttgtatgcgcgattatcccccatgtaatcagctggggtctttccctgaccttggctcctacaaatctcttggcggatgtatccccttggcagtttgctgggggtcgtacggtccaggttcaatccatggcccaaggggcgctgtggttcctacagtgggatcacctcattggaatggggagctttctcctgtgggcactgcatatgcggcggacagttgagaggcagtcgtcattcacacagacttgctatctagcgctgaaagttggaggtctttgtctgatctcagggccctgcggtgcggctgtttggttgctgcgggaggaatctcaattctga

SEQ ID NO: 8 - DNA, 1053 bp, Clitopilus passeckerianus
  source 1..1053: mol_type = genomic DNA
atgagaatacctaacgtctttctctcttacctgcgacaagtcgccgtcgacgccactctgtcatcttgctctggagtgaagtcacgaaagccggtcattgcctatggctttgacgactcgcaagactctcgcgtcgatgagaatgacgaaaaaatattggagccctttggctactatcgtcatcttctgaaaggcaagagcgccaggacggtgttgatgcactgcttcaacgcgttccttggactgcccgaagattgggtcattggcgtaacaaaggccattgaagaccttcataatgcatccctactaattgatgacatcgaagacgagtctgccctccgtcgtggttcaccagctgcccacatgaagtacgggattgcgctcaccatgaacgcggggaatcttgtctacttcacggtccttcaagacgtctatgaccttggcatgaagacaggtggcacacaggttgccaacgcaatggctcgcatctacactgaagagatgattgagctccatcgcggtcagggcatcgaaatctggtggcgtgaccagcggtcccctccctccgtcgatcaatacattcacatgctcgagcagaaaaccggcggcctgctcaggcttggcgtacggctcttgcaatgccatcccggtgtcaatagcagggccgacctctccgacattgcgctccgtattggtgtctactaccaacttcgcgacgactacatcaacctcatgtccacaagctaccacgacgagcgtggatttgctgaggacattaccgaaggaaagtataccttcccgatgttgcactctctcaagaggtcacccgactctggactgcgtgaaatcttggaccttaagccggccgacatcgccctgaaaaagaaagctatcgctatcatgcaagagacaggatcgcttgttgcaacccggaaccttctcggtgcagtcaggaatgatctcagtggattggttgctgaacagcgtggagacgactacgctatgagcgcgggtcttgaacgattcttggaaaagttgtacatcgcagagtag

SEQ ID NO: 9 - DNA, 2880 bp, Clitopilus passeckerianus
  source 1..2880: mol_type = genomic DNA
atgggtctatctgaagatcttcatgcacgcgcccgaaccctcatgcagactctcgagtctgcgctcaatacgccaggttctaggggtattggcaccgcgaatccgactatctacgacactgcttgggtagccatggtctcccgtgagatcgacggcaagcaagtcttcgtcttcccggagaccttcacctacatctacgagcaccaggaggccgacggcagttggtcaggggatgggtcactcatcgactccatcgtcaacactctggcctgccttgtcgctctcaagatgcacgagagcaacgcctcaaaacccgacatacctgcccgtgccagagccgctcaaaattatctcgacgatgccctaaagcgctgggacatcatggagactgagcgtgtcgcgtacgagatgatcgtaccctgcctcctcaaacaactcgatgcctttggcgtatccttcagcttcccccatcatgaccttctgtacaacatgtacgccggaaaactggcgaagcttaactgggaggctatctacgccaagaacagctccttgcttcactgcatggaggcattcgttggtgtctgcgacttcgatcgcatgcctcatctcctacgtgatggtaacttcatggctacgccatctaccaccgctgcatacctcatgaaggccaccaagtgggatgaccgagcggaggattaccttcgccacgttatcgaggtctacgcaccccatggccgagatgttgttcctaacctctggccgatgaccttcttcgagatcgtatggtcgctcagctccctttatgacaacaacctggagtttgcacaaatggatccggaatgcttggatcgcattgccctcaaactacgtgaattccttgtggcaggaaaaggtgtcttaggcttcgttcccggcaccactcacgacgctgacatgagctcgaagaccctgatgctcttgcaagttctcaaccacccatatgcccatgacgaattcgtcacagagtttgaggcacctacctacttccgttgctactctttcgaaaggaacgcaagcgtgaccgtcaactccaactgccttatgtcgctcctccacgcccctgatgtcaacatgtacgaatcccaaatcgtcaagatcgccacctacgtcgccgatgtctggtggacatcagcaggtgtcgtcaaagacaaatggaatgtatcagaatggtactcctctatgctgtcttcacaggcgcttgtccgtctccttttcgagcacggaaagggcaaccttaaatccatatctgaggagcttctgtccagggtgtccatcgcctgcttcacaatgatcagtcgtattctccagagccagaagcccgatggctcttggggatgcgctgaagaaacctcatacgctctcattacactcgccaacgtcgcttctcttcccacttgcgacctcatccgcgaccacctgtacaaagtcattgaatccgcgaaggcatacctcacctccatcttctacgcccgccctgctgccaaaccggaggaccgtgtctggattgacaaggttacatatagcgtcgagtcattccgcgatgcctacctcgtttctgctctcaacgtacccatcccccgcttcgatccatcttccatcagcactcttcctactatctcgcaaaccttgccaaaggaactctctaagttcttcgggcgtcttgacatgttcaagcctgctcccgaatggcgcaagcttacgtggggcattgaggccactctcatgggccccgagctcaaccgtgtcccatcgtccacgttcgccaaggtagagaagggagcggcgggcaaatggttcgagttcttgccatacatgaccatcgctccgagcagcttggaaggcactcctatcagttcacaagggatgctggacgtgctcgttctcatccgcggtctttacaacaccgacgactacctcgatatgaccctcatcaaggccaccaatgacgacttgaacgacctcaagaagaagatccgcgacctgttcgcggatccgaagtcgttctcgaccctcagcgaggtcccggatgaccggatgcctacgcacatcgaggtcattgagcgctttgcctattccctgttgaaccatccccgtgcacagctcgccagcgataacgataaggctctcctccgctccgaaatcgagcactatttcctggcaggtattggtcagtgcgaagaaaacattctccttcgtgaacgtggactcgacaaggagcgcatcggaacctctcactatcgctggacacatgtcgttggcgctgacaacgtcgccgggaccatcgccctcgtcttcgccctttgtcttcttggtcatcagatcaatgaagaacgaggctctcgcgatttggtggacgttttcccctccccagtcctgaagtacttgttcaacgactgcgtcatgcacttcggtacattctcaaggctcgccaacgatcttcacagtatctcccgcgacttcaacgaagtcaatctcaactccatcatgttctccgaattcacaggaccaaagtctggtacagatacagagaaggctcgtgaagctgctctgcttgaattgaccaaattcgaacgcaaggccaccgacgatgggttcgagtacttggtcaagcaactcactccacatgtcggtgccaaacgtgcacgggattatatcaatatcatccgggtcacctacctgcacacggcactctacgatgaccttggtcgtctcactcgcgctgatatcagcaacgccaaccaggaggtttccaaaggtaccaatggggttaagaaagctaatgggtcggcgacaaatgggatcaaggtcacagcaaacgggagcaatggaatccaccattga

SEQ ID NO: 10 - DNA, 1572 bp, Clitopilus passeckerianus
  source 1..1572: mol_type = genomic DNA
atgctgtccgtcgacctcccgtctgttgcgaacttggatcccgtgatcgtggctgctgctgcaggatccgctgttgccgtctataagctccttcagctaggctccagggagaacttcttgcctcccgggccacctaccaagcctgttctcggaaatgctcatctcatgacgaagatgtggcttccaatgcaattgacacagtgggccagggagtatggcgaagtgtactctctcaaattgatgaatcgcactgtgattgttttgaacagtccaaaggctgttcggactattcttgacaagcagggtaatatcacaggagaccggccattttcgcccatgattgcccggtatactgaaggcttgaatctcacggtggaaagcatggacacttctgtatggaagactggtcgcaaaggtatccacaattacctaacgccaagtgccttgagtggctacatagcgcgacaagaagaggaatctgtgaacctcatgcgcgatctattgatggacgctcctaatcggccgatccatattaggcgcgctatgatgtcgctgctcctacacattgtgtatggccagccacgctgcgagagttactatggcacaattatcgagaatgcatacgaagctgccaccagaattggtcaaatcgctcacaatggtgcagcggtcgacgctttccccttcttagactacattcctcgcggtttccccggggccggctggaagactattgtggacgaattcaaggattttcgtaatggtgtctacaattctctcttggacggtgccaagaaggcgatggattccggtgtcaggaccggatgttttgcagagtccgtgattgaccatccggatggtcgtagctggattgagttatcaaaccttagcggtggcttcttggatgccggcgcgaagaccacgatatcgtacatcgaatcgtgtattcttgctcttatcgcccacccggactgccagcgcaagatacaggacgagctggataatgttttggggaccgaaaccatgccgtgcttcaatgatttagagcggttgccttatctcaaggcgttcctacaggaggtccttcggattcggccagtcggccctgtagcccttccccacgtctcgcgggagagcttgtcttatggcggttacgtactaccagagggaagtatgatcttcatgaacatctggggaatgggccacgaccccgagctcttcgacgaacctgaggccttcaagcctgaacgctatttcttgtcgccaaacggcacgaagccaggcttatctgaagacgtcaatcccgatttccttttcggtgctggacgtagagtctgcccaggcgataagctcgcgaaaagatcaactggtctcttcatcatgaggctctgttgggcattcaatttttacccagattcttcaaacaaggacactgtgaagaatatgaacatggaggactgttacgacaagtcggtttctcttgagactcttccacttccgttcgcatgcaaaattgaacctcgagataagatgaaggaagacttgattaaggaagcgttcgctgcgttgtag

SEQ ID NO: 11 - DNA, 1578 bp, Clitopilus passeckerianus
  source 1..1578: mol_type = genomic DNA
atgaatctttctgctctgaaggctgctctgcttgacagcaacatgatcgcacctgtggccatccctttggcatgctacttggtctacaagctgcttcgtatggggtcgagggagaagacgttacctcctgggccacctacgaagccggtgttgggtaatctccaccagatgccagcaatggacgacatgcaccttcagcttagccgatgggcacaagaatatggaggaatatacagcttgaagatcttcttcaagaacgttatcgtcctaacagactcagcctccgttactggcattcttgacaagctgaatgccaagactgctgaaagacccactggtttcctccctgctcctatcaaagacgaccgtttccttcctatcgcctcctacaaatccgacgaattccgaatcaaccacaaggcctttaagttgctcattagcaacgacagtattgatcgatatgcagagaacattgagacggagaccatcgtgctgatgaaggagctgttggctgagcccaaggaattctttaggcatctcgtccgcaccagcatgtccagtattgttgctatcgcttatggtgaacgcgtcctcacctcctcagacccattcattccctaccacgaagaatatcttcacgacttcgaaaacatgatgggtctccgaggtgttcacttcaccgctctaattccttggctcgccaagtggcttcctgatagtctggccggctggagggtcatggctcaaggtatcaaggacaagcaacttggtatctttaatgatttcctcggaagggttgagaagagaatggaagctggcgtcttcgacgggtctcacatgcagaccattcttcagaggaaggatgagtttggattcaaggatagggatcttattgcctatcacggaggcgtcatgattgacggaggaactgataccctcgctatgttcactcgtgtcttcgtgctcatgatgacgatgcaccccgaatgccagcagaagattcgtgatgagctgaaggaggtcatgggcgatgaatacgactcgcgtttgccaacttatcaagatgcattgaagatgaaatacttcaattgcgtcgtcagagaggtaactcgcatctggcctccgagtcccatcgtaccgcctcattactcgacagaggatttcgaatacaatggctacttcatcccgaagggtaccgtcatcgtgatgaacctttatggcatccaacgagacccaaatgttttcgaggccccagacgatttccgccccgaacggtacatggagtctgaatttggcacaaaaccaagcgttgacctgactggctaccgtcataccttcactttcggcgctgggcgcaggctctgtcctggactcaagatggctgaaattttcaagcgcactgtatctttgaacatcatctggggattcgacatcaagcccctgcctaacagccccaagtcaatgaaggacgatgtcgttgtacccggtccggtctcgatgccaaaaccgtttgaatgcgagatggtaccacgtagtcagtcagttgtgcaggtgatccacgatgttgcagactattag

SEQ ID NO: 12 - DNA, 762 bp, Clitopilus passeckerianus
  source 1..762: mol_type = genomic DNA
atggaaggcaaggtcgcaatcgtcactggcgcatccaatggtattggactcgccaccgtcaatctcctcctcgcagcaggagcgtctgtctttggtgtagacctcgctccagcaccgccctcggtgacctccgagaaattcaaattcctacaactcaacatctgcgacaaggatgcacccgctaggatcgtatccggctccaaagaggcctttggcatcgagaggattgatgccctcttgaatgtcgctggtatttcggactacttccagactgcgttgaccttcgaggacgatgtatgggaccgagtcctcgatgtcaacctggctgcacaagtgaggttgatgagagaggtattaaaggtcatgaaggtgcagaaatcggggagtatcgtgaatgtcgtcagcaagctggccctcagcggtgcttgtggtggtgttgcatacgttgcgagtaaacatgccttgcttggcgtgacgaagaacacagcctggatgttcaaggatgacggcattcgatgcaatgcagtcgcacctggttcgactgacaccaacatccgaaacacgacagacccgtccaaaatagattacgacgccttctctcgagccatgcctgttatcggcgtacactgcaacttgcaaacaggtgagggcatgatgagccctgagcctgcagcccaagcgatcttcttcctagcttcagacttgagtaagggcacgaacggtgtcgttattccagtcgataacgggtggagtgtcatttag

SEQ ID NO: 13 - DNA, 1569 bp, Clitopilus passeckerianus
  source 1..1569: mol_type = genomic DNA
atggctccgtcaacggaacgtgctctaccagtccttgtaatatggactgctataggcttggcctactggatagattctcagaagaagaaaaagcagcacctgccgcctgggccaaagaaacttccaattattggcaacgtcatggacctaccagcgaaggtcgaatgggaaacctatgctcgctggggtaaagagtacaactctgatatcatacatgttagcgccatgggaacctcgatcgtaatactgaattctgccaacgccgccaatgacttgttgctgaagaggtcggcgatctactcgagcagaccacacagcacgatgcaccacgagctgtcaggatggggctttacgtgggccttaatgccatacggcgagtcatggcgggctggtcgaagaagcttcaccaagcacttcaactcttcaaaccccggtataaaccaacctcgtgagttgcgatatgtgaaacggttcctcaagcagctttacgagaagcccgacgacgttctcgatcatgtacggaacttggtcggctctacgacgctttcaatgacctatggccttgagactgaaccttataacgacccctatgttgacctggtcgagaaagctgtccttgcagcgtctgagattatgacgtctggcgcctttcttgttgacatcatccctgcgatgaaacacattcctccatgggtcccagggactatcttccatcaaaaggctgccttaatgcgaggtcatgcgtactatgttcgtgaacagccattcaaagttgcccaggagatgattaaaactggcgattatgagccctcctttgnatctgacgctctccgagatcttcagaactcggaaaaccaggaggcagatttggagcacctcaaggatgttgctggtcaagtctacattgctggtgctgatacgactgcatccgccttggggactttcttcctcgccatggtctgtttccccgaagtacagaagaaagcacaacgagaattagatagtgttctcaatggaaggatgcccgagcacgccgacttcccctctttcccatacctcaacgctgtgatcaaggaggtttaccgctggagacctgtgactcctatgggcgtacctcatcaaaccatctcagatgacgtttacagggaataccacatccctaagggatccatcgtgtttgccaaccaatgggcgatgtccaacgacgagaccgattacccccagccagacgaattccggcctgagcgatacttgaccgaagacggtaagcctaacaaggctgtcagagacccctttgatatcgcattcggcttcggtagaagaatttgcgctggtcgttacctcgctcattccaccatcaccttggctgcggcctctgttctgtcgctgtttgatctcttaaaagcagttgacgaaaatggcaaagaaattgagcctactagagagtatcaccaggctatgatctcacgtccactagatttcccttgccgcatcaagccaagaagtaaggaagctgaggaggtcatccgtgcttgcccgttgacgttcacgaagcctgctagtggctag

SEQ ID NO: 14 - DNA, 1134 bp, Clitopilus passeckerianus
  source 1..1134: mol_type = genomic DNA
atgaagcccttctcaccagaacttctggttctatctttcattctattggtactatcttgtgccatccggcctgctagaggacgatgggttctctgggtcattattgttgggctcaacacctacctcaccctgactccgaccggcgattcgaccttggattatgacattgccaataacctcttcgttattaccctcacggccacagattatattctcttgacggacgtccagagagagttacaattccgcaaccagaaaggtgtcgagcaagcctcgttgcttgaacgcatcaagtgggcgacctggctggtgcaaagtcggcgtggtgtgggctggaattgggagccgaagattttcgtccacaagtttgacccaaagacttcacgcctttcattcctcctccagcaactcgtcacaggttttcggcattaccttatttgcgatctagtctcgctatatagccgcagtccagtcgccttcatcgaacctcttgcttctcgccctctgatctggcggtgtgcagatattaccgcatggctcctgttcacgacgaaccaagtatcaattcttcttacggcattgagtgtcatgcaagttctctcaggttactcagaaccacaggactgggtccccgtgtttggccgctggagagatgcttataccgttaggcggttctggggtcgatcgtggcatcaattggttcgcagatgcctatcagccccaggaaaacatctttccacgaagattctaggcttgaagtctggctctaacccggcgctttacgtacaactgtacaccgcattcttcctctcgggagttttgcatgcgattggggacttcaaggttcacgcagattggtacaaagccgggactatggagttcttctgtgttcaagcggcgatcatacagatggaggatggggttctctgggtcggaaggaagcttggtatcaagccgacttcgtactggaaggcccttggacatctttggactgtggcatggttcgtctacagctgcccgaattggctgggggcaactgtctcgggaaggggaaaggcctcaatgtcgttggagagtagtctcattcttggtctgtaccggggggaatggaatccccctcgtgtagcacagtag

SEQ ID NO: 15 - DNA, 1704 bp, Aspergillus nidulans
  source 1..1704: mol_type = genomic DNA
atgggaacagatttacatctcctgcagccggcagctttagacttaccgttgcggccctcccagtatgctgctggtcctattcccagtctgaaagattttcaggcacagtggaccgcatgggatatagtcaccaaagcaatgatcccacaagaagagctgctttcaaagccgattaaactacgaaattccctgatattttacctaggacatattcccacgttcgctggtaagttctatcgcagacggacttcaattgggctattgcttactcaagctttcagatattcacttgacccgtgctttgggtggcaagcctacagaccctaaatcatatcaactgatatttgagcgcggcatcgaccccgacgttgacgatccggaaaagtgccattctcacagtgaaattccggacgagtggccatctcgttcagaaatcctagattaccaagaccgggtcagaaacaggatacgatccacactcgagatacccggcctgtccaagaacagaacgctgggtgaagccctttggatcgggtttgagcacgaggcaatgcacttggagaccttcctctacatgctgatccagagtgagagagtccttcctccgccgggtatccaggcgccggatttcaagaagatatttcttgacgcaagagaaaatgcgaagccgaacgaatggttcgctataccagaacaaaccctatctatagggcttgactcttcagaggacttgatgccaacgtcaacattcggttgggataacgaaatacctaagcgcaatgtaacggtacaaccatttgaagcgcaagggaggcccgttaccaatggtgaatatgcccggtaccttcagaagaaccaaattcacaggtggcctgcctcctggattatgaccaatcaagcagatggagttgcgaacggtagcaccgcaactgaggattttctaactgacttctctgttcgcacggtcttcggccccgttccacttgagctagctcaggactggccagtcattgcatcctacgacgagcttcaacaatacgcttcatggatgaattgccgcctgccgacgttcgaggaggccaaaagcatctatgcacactcagctcgtctaaagaaaactcttacagacggctccgcggatgcagctagcaacggactcaaaaacggctaccacaaaccgcatcttagaaacggcaccgtcaaaattgctccttctacagcccaacccgtcctcccgccctctgcatccgcatcctctccacctgtttttctcgacctcacgacttgcaatgtcggtttcaagaactggcatcccatgcccgtgatccagaatggcgataaactggctggccacgcagagctcggcggcgtttgggagtggacttcgtcacctttggagccccatgatgggttcaaagccatggagatttacccaggctatacttgttagttttccttctaggcacactcgtctgcatcattcactgacttattctcttcctagccgacttcttcgacggaaagcataatataatcctcggtggttcgtgggcgactcatccgcggattgttgggaggacgacgttgtaagtcccattatcgtattcagttcctggaaaaggctaatacagatgtagtgtgaactggtatcaacgaaattatccttacgcatgggctggagcgcgacttgtacgggacgtttga

SEQ ID NO: 16 - DNA, 1520 bp, Aspergillus nidulans
  source 1..1520: mol_type = genomic DNA
atggcctctccaactccgtttggcgcccccatgaaagcgcacttcctcctcgatcctaattacaacaatctcaaccacggtctttcctctccttcaacgtcgcttacagcaattgctgacacaaccaggctctttcggcacctacccttcccaggtccttgaaaagcaacagtccatccagaaatctctcgaatctaggcccgacatcttcatccgttacattcagcccggccttatcgacacttctcgggccgccctcgcgcccctccttaacgtccccgtctcagacctcgttttggtcaaaaatgccacgacaggtgtcaacactgtcctgcacaatctcgcactgacccgtactctgactgcagatgacgttatcttctactttgacaccgtctacggcgccgtcgagcgcgccctctttgccctaaaggaatcctggggcgtcaagctgaggaaggttaagtatgttttcccccttgaagaggggggaatggtcaagaggtttagggaggcactgaagagcgtgagaaaagagggcttgacgccgaagctcgcggtctttgagacggttgtgtcaaacccaggcatcaggtttccctttgaagaaattacgagggcttgtaaggaagagggtgtgctgagtcttattgatggagcacatgcggttggtatgatcaagctggatctggccgcgttaggggtggacttcttcacgagtaattgtcataagtatgccctgtctctcccttttcattaccctagcgcaggcttgatggtttatgcttaggtggctctacactccgcgctcctgcgcagtcttatacgttcccgagcgcaaccagaagttcatccggacaagtctccctacgtcatgggggtatgtgccgccacaggttccaccttctgaatcaggcgaagacaaggatatccctccgtccacgctcccaaatactggaaaatccccgttcgtcgcgctctttgagttcacagggacaacggacgacagcgcgtatgcgtgtgtccctgcagcactcaacttcagagatgaggtctgcggtggcgaggagaggatctatgcttaccttgaacgactcgctggggaggcgggtgaactccttgcgtctgcattagggacggatgtgttgagggctgtgaaagatgcaagcggtgcagggaaaggtggaaaggggctgggttgctcaatggtcaatgtgcggttgcctattcggatcactaatctggacttaaattctggaacgaaggataaggcgataaatgtacggccggaggacgtcagtcccctgtcgcactggctacacgagcagttgatagccagagggacattcgtaccgtgttttccgcatgggcagtggatgtttgtcagactcagcgcgcaagtttacctggaaaggagtgattttgtttggttgggtgatgtcttcaagcatataatggagggtgtaccgggctttttggaggcgaacaagaatggaactcaggccaaagcgaagatttag

SEQ ID NO: 17 - DNA, 2890 bp, Aspergillus fumigatus
  source 1..2890: mol_type = genomic DNA
atgtccccgttgccgtgtccttcgaaaaaggtcgagattgtcgatatacatcgaaatgacgtggaattctcgctcgtgaatgagattcgcaagggcctcaaccctcccgagggtactccaagatcattgcccaccatgctgctctacgacgctcaagggcttaagctcttcgaggagatcacctacgtggatgagtactacctgaccaacgcggaaatcgaagtgttgcagaaccactccaagaagattgtggaacgcgtccccgagaacgcacaattattggagctgggtagtgggtatgatctctcctgaagaatgctctgacggttctgtatctaatcatccgtgcaggaaccttcgtaagatcaagatccttcttcaagagttcgagcggacaggaaagcacgtcgactactatgcgttggatctatccctgtcagagctacagcggacctttgtcgaggtatcatctgacgagtactctcacgttgatctccacggtcttcacgggacctatgatgatgcccttgcctggctgagcaatccccagaacctacagcgtcccactgtggttatgtctatgggatcctcaattgggaatttcagtcgtgaaggtgcagcagagtttcttgcccaatttgctcggctgctcaagccatccgatctgatgatcattgggctagatgcttgtacagacccggacaaggtctacaaggcatacaacgattcaaagggcattactcagaggttctacgagaatgggctattacatgcgaatgcagtccttggatacgaggcattccaactttcggaatgggaggtggtgacagactacgacgttgccgggggccgacatcgagcattctactcaccgaagcagaatgttacaatcgatggcgtgcttctgcaaaaaggcgagaaattggtttttgaagaggcaacaaagtatagccctcaacaacgggagcaactctggcgggatgcaaacctcgtcctttgtgaggagcttggaattagctctgaggaataccgtaagttggtccttgcctcccgcagcgctgttgtagcagagatgtatgggagaagcgtcgactaatgaacgacccttcactgacagatatccatctgctgtcaccgccaaccctcagccttcccagtcaaccgtccgaatatgctgccaatccagttccgagcttcaaagaattccagtcattgtggaccgcttgggacactgtgacaaaggccatggtgccccgcgaagaactcctcgcgaaacctatcaagcttcgcaatgcattgatcttctactttggacacatccccacattcacgggtcagtaatccagagacttttattcaacgccgtgagcatgatactgatgctctattagacatccacttgacacgggctcttggtggctctccaacagaaccacgaaactatcgacagattttcgagcgtgggattgaccccgacgtagacaatcccgagcactgtcactcccatagtgagattccggatgaatggcctccccttgctgagatactggattatcaggatagagtcagaagtagggttgattcggttttgcagagggatgacataacacagaaccgctgcctgggggaagcactctggattggcttcgaacacgaagcgatgcacctagagacctttctctatatgcttttgcagagtgacaagaccctcccaccgccccttgcagacagaccagattttgagaagttgttccatcaggccagagcgaacgcgaaaccaaatgaatggtttgctataccagaacagacattatccatcggttttgacgatactgacgaacaatctttgcctgatgtgtcttttggatgggacaatgagaagcctcagcgaacgatcacagttcgtgctttcgaagcacaggctcatgctataacaaacggcgaatatgccaaatatctgcaagcgactcgtcagcgccggcggccagagtcgtgggttctaactcattcggatgagaattatccaatctccaagggggtgactctggagagcagccaagccacaaaggatttcatggacaacttcgctgtccgcaccgtctttggacctgttccgcttgaattcgcgcaggattggcctgttatggcttcctatgacgaactggccctatacgccgaatgggtgggctgcagacttcccacttatgaggaggtcaagagtatctacaactactccgctcagctgaaggagacaagacaacatgagccatcagatcacgagaggttcgtaaattcccctcggtctttaagtttagctatgtctgacagaaggcagcaacggtgtgaagggcattaaccgtgatatggtgacgaacgggcactcgaaggttcaccaggataagccccggaccccagaacgtcagccaatccaacctccttcccaaagcacaatgccagtatttgttgacctccacggttgcaacgttgggttcaagcactggcaccctaccccggtcatccagaacggcgatcgactcgccggtcacggtgagctgggaggagtctgggaatggaccagcacgccactgacgccacatgatggcttcaaggccatggatatttatccaggttatacaggtaagctgttgacctacttggtggatttctataaacccaactaaccagcaatagcggacttctttgatggaaagcacaacattgtcctgggtggttcatgggccacgcatcctagaattgcgggacggacaacattgtaagttatcacctgtgatgtcgacctgaatatgcactaactgtctcaagcgtcaactggtatcagcacaactatccttacacttgggcgggagcacgtctcgtgcgcagccagtag

SEQ ID NO: 18 - DNA, 7537 bp, Aspergillus fumigatus
  source 1..7537: mol_type = genomic DNA
atggggaggctccagctctcaagcgggctgaaggccatagccctgctcacattcgcagcgacagcaacatgctggccatacgacgagtccctcgttgactacaacgtcaacacgaataagtcggccactaaccccgccgactactggggagaatggtcggatcacaagtaccatccgtcgccagagaactggcggtttccgttttacacactcttcatggacagattcgtgaatggggatccaacaaatgataacatcaacgggaccacgtttgagcacgatctcaattcaaatcagatgcgtcatggcggtgatgttgctgggctggttgatacgctggattacttgcaagggatggggatcaaggtgcgttgccgctattattcgtcaatttctggtaaagatatagttgatgctaacgatgcgatagggactgtaccttgctggaactccgctcatgaaccagccctggggctcggatgggtattcggcgctcgacaccacccttctcgaccagcactttggcacgatccaggtctggcgcgacgccatcaccgaaatccacaaacgggggatgtacgttctgttcgacaatacaatcgctacgtgagtacctcgcctccctgcgttttgccatcggtttgtaaaaaacttgggcctgacactagattactgcagcctcggtgatctcattggttttgaaggtcatctcaacgacaccacgcccttctccgagaaagaacacaaggccctttggaagtccaacagacggtacgtcgatttcgatattggcaacacctacaacgccacctgtgattatccgcgcttctggtacgaggacgggatgccagtcaatgagtccctgaccgcgggcctggtcgggtgttatgatagtgactttgaccagtacggtgacatcgaagcgttcggtgtctggccagactggaagcgtcaactggcgaagtttgcgtccgtccaggatcggttgagagaatggtacccacctgtacgggaacggctgatccgacacacatgcatgatcattgcctcctttgatatcgacggtattcggtacgacaaggcgacccaggcgactgtcgatgcgttgggggacatgtccaaagcgtatcgggaatgcgcgcgggctgtcggcaaggagaactttttcattgcgggtgagatcacaggaggcaatacttttgggtctatctatctcggacgaggacggcagtcgaaccaggtcgattcggtggggaatatctacgacgccatgaaactgacaaacgagtcggatccgcagcttttcctgcgcgaggtcggccacgaggctatcgacgccggtgccttccattactcgacttaccgtgccctgacccgcttcctgggaatggacgggcagctggaggccggttatgacgtccctcttgactgggtgcaggcatggggaaacatgaccgtgaccaacgacctgattaacgccaacacgggcaagttcgatccccggcacatgtacggcgtgacaaaccaggatgttttccgttggccggcaatcgagtggggtgttgagaggcagatgctgggctcgtttatcaccacgctgatgctgccgggcatcccgctgctgctttggggagaggagcaagcgttctacgtgctcgatgcgacggcgtcaaactacatatatggacgacaggccatgtcgtctgcgaccgcgtggaagacgcacggctgcttctcactcgaatcgagccagtactaccagtggcccctggtggccgcactcgacggttgcaacgacgagacggtcacctacgaccaccgggacccgagccacccggtgcgcaacatcatcaagcacatgtaccagatgcgcgagcagtacccaatgctcaacgatgggttcatcatcgagaccctttctaaccagacggaacctgtctactaccccggttccaacgggaccgagaccgagacaggcatgtggtcggtccgccgtgaccggaacgaagagacccaggactttggctcgagtgacgacaacgaacccatctggctagtctatagcaacatgaaccgcacgcacgactacacatttgattgctctgacaatgagacggcactcattgccgccttcccctcaggcaccaaggtcaggaacctctttcacccgtacgacacgctgaccctaggcgacgggccaaaggaaatggtctatggcaactcgactgagctggtaggctgcttaccgaacctgacgcttagccggtacgagttccgtgcctatgtcaagaacgagctctggaaaaagccccggccgatgatcaccaagttccagcccggcgacgatgaggccaacggccacgacagcccgctgcgttcgaccgtcgcaccagatgcgtccgagacggtgcgactgacgctgcagttttctgaggcgatgggatgcgattctgtcacagactccatctcatttaactcttccacggaaaccggcaagattccatccatcgacgcctccaccgtccagtgtgggaacatcactgaagtcgccaacagcaacgctaccgggcacatccccggtaagtggcagtgggctgcagatctgagaggggtgtacaacggcatccaccgggtcactgtcaacaacgccagcaacgccgacggggacgactccacccacgcagtcgaccacttcctcttccggattgggcagattgacaaccccatggtctttacctctgccaactactccagcagtttgctgcatgagaaggaggacggcacgttctatatccaacaccacgctgccggtgctgacaagtaccgctattcgacgaattggggcaccacgttctctgactggaaaacgtacaagggaggtaacgacacgattaccatgcttccatggaatggcactaaggcgcaggaatgggagggacatcacatccgcgtcgagtactggtcccgctggactggcagcagtagccacgtccaggaaggcgattccggctggaagtacaagacgccacgtcgcttcccacatgcctttttcaatgggccctacaaccagtacgggtacgacggcggtctggacaaccagatcaagctggacgcgggcgccggcggcgacggatactggaagtaccatttcacctcggagtggcctgccgtcgggcaggtgaatgtctggggcatcaatccggatggcgagcccgatcagagctgggtgatgggcgacgtggacggggacaaggtcctcgaccgcatgccaccaagcgcgctctctgcaacgctgattaacatcactgatcacccaacgcacccttatatctcgtggaagctgtacataaacgacgcgacgatgcggtactatctcatccccgctggccaccagagcgggcagatcgccatgttcgtcctcttctggatcatccctctcctgtccggctccgcctgcgtctatatcttcatgaagtccttctacaaggtcaaattcaacgaaatcggcgctgccggcgcaagcacggaaatgaagtcacttgtcccgcttgctctccgtcgacgcatgaagcagctcgcctctgggaatggcaagaacggtccgtcattcaacccgctcatgcgccttgctgaaaagtccggctttatgcagagcacgactgcgttggccggtgcggcctcgggcaagagacgcatggtcctgatcgcgaccatggaatacgatattgaagactgggggatcaagatcaagatcggtggtctgggagtgatggcccagctgatgggtaagacactcggccaccaggaccttatctgggtggtgccttgtgtcgggggcgtggactatcctgttgatacccctgcggaacccatgacggtcactatcctgggtcaggcataccaggtgaacgtgcagtaccacgttctgaagaacatcacgtacgtcttgcttgacgcgcccgtcttccgccagcagaccaagtcggagccgtacccagcgcgcatggatgacttggactctgcagtgtattactccgcatggaatcagtgtatcgcagaggcgatcaaacggttccctgttgatttgtaccatattaacgattaccacggctctgtcgcgccgctgtacctcctgccaggtaccatccccgcctgtctgtcacttcacaatgccgagtttcagggtctatggccgatgcgaacacagaaggagaaagaggaggtctgttccgtgttcaacctggatgttgaggttgtccgcaactacgtccagtttggtgaggtgttcaacctcctccacgcaggagcgagctatctccgcgttcaccaacagggctttggtgccgtcggtgtctcgaagaagtacggcaagcgttcatatgctcggtacccgatcttctggggcctgcgcaagatcggaaacctgccgaaccccgatccgtctgatgtgggcgagtggacgaaggaggacagcctgatcaaggacgaggatatcaaggtggatccagagtttgaagcaggccgcgctgagctcaagagacaggcgcaggagtgggctgggctggaccagaaccctgatgctgacctcctggttttcgtcggtcgttggtccatgcagaagggtgttgaccttatcgcagacgttatgcccgcagtccttgaagcacgaccaaacgtacagctcatctgtgttggacctgttatcgatctctatggccgctttgcggcgctcaagctcgaccgcatgatgaaggtgtatcccggccgcgtcttctcacgccctgaattcactgccctgccgccgtatatcttctccggcgcggaattcgcgcttattccctcccgtgacgaaccctttggtcttgtcgccgtcgagtttggccgcaaaggtgcgctcggcattggagcccgtgttggtggactgggacagatgcctggatggtggtacaatgtcgaatcggtctcgacatcccatctcctcatgcagttcaagctcgctattgaggctgccctttcgtcaaagactgagacacgtgctatgatgcgtgctcgctctgctaaacaacgcttccccgttgcgcagtgggtggaagatctggagatcctacagtcgactgctatccaggttcatgagaaggaggtttcccgtggtcatgcaggtggccgccccatgacaccgatgacgccctctggagctacgacgcccagcggaatgatgacgcctactacaggatcccgtggtcttaagcctctgtcccagggcgtcggcatgggcctctcggtgccacactcccgggagagcagctattcgaacctcaaccgattgagcgagtacgttgcgcaaaagactccgggcgagtctcaaccgcgagaatcgtccggtctgcagcgctcgctctcgctcggtgtccggtccggtcctggccaccgtggtcgtgcgcgcaagcagaaacccggcgcggacaacatcccggagggcaacgaggacggcagcagcagtgatactgagtctatccctgactactacgacgacgagtacaccctcaccccagctcagattgaagaaagcagacgggctcaggccacccgttccatatccttcagccccgagacactccagccacctcgatctcccctgcccgcacctcccatgagtcccgggacgccaccgtcggttgagcagactctgctgcctccaccaaagcccttcgctgccgctgacgctggaaacagacttagtagtgcatcagtgttatccctagactctgtcgtgggtgggaagaaagacttcaagctccaaaaggtcgaccccttctttactgacagcaacggcgagtatgctcggaacttcgaacagcagctcgagaacctcaacggctccaactccgagtcacagctgtgtatcgaggagttccttgtcaaatcggaacgccgctggttcaacaagttccgcgatgcaaggctgggtcgtctgcgctcgcccacaccgtctgtcttccgcgacaaccacagccacggccggggctcgcccgatggatctatgtatgtggatgaggcaggccaccgtaatagtggcgatgccgtgcacgacaacggctcagatgacacagacgatgagttcctcctaggcaaagactacgtgcctcccacgggcctgaagaaatggatgcagatcaagatcggcgactggccggtgtacacgctcttcctcgcactgggccagatcatcgctgcaaactcctaccagatcacgctgctgacaggcgaagtcggccaaacagccgagaaactgtacggaattgccacaacctatgccataacctcagccctctggtggctcgtcttccggtacttcaaatccatcgtctgcctctccaccccctggttcttgtatggaattgcgttcctcttcattggctccgcgcactttgagtccgactcattcacccgcggctggatccagaatgtcggcagcggcttctatgctgcggcttcctccagcggatccatcttctttgcgctgaacttcggtgatgagggcggtgcaccagtgagcaagtggattttccgtgcatgtgtgattcagggcatccagcaggtctatgtcattgtgctctggtattggggttccacgatggcacaccagtccagccagggtctgctcacggcggataatacgatctcaaatacgtggaagatgacgtacgtccaaaacgaccctaaattacccccaattaatatacagaactcactaacaaactgatacagcgccatttgctatccgatcgccatgttactgtgggccatcggcctcctgctgatcttcggcctaccaaactactaccgccagaaacccggcaaagtaccctccttctacaaatcccttttcagacgcaagatcgtgctctggaactttgtcgccgtcatcctacagaacttcttcctgtcggctccctatggccgcaactggagtttcctctggacatcctcccacacaaagccctggcagattgtcatcctgtgtgtcatattctttggtcttctgtggtgcgcgttcctttacattgtcgctgttctttcgaagcagcactcctggtttttgccagtcttcgcatgcgggctcggcgccccccgcttcctgcaaatctggtggggtgtttctggcatcggacactaccttccctgggttgccggcgggtatactggcggcgcactagttagtcggagtatctggctgtggctaggtgtgctagattcaatccaggggttagggttcggaatcattctcctccaaaccctgacccggatgcatatgtgctttacgttgatcgtcagccaagtcctgggatctatagcaaccatcgttgcgagggcatgtgctccaaataatgttggtccgggacctgtgagtccagatatcacgaagggagcgggcgaactggcgaacgcctggttctgggttgcattgttctgtcagttgcttgtctgtgctgggttcttactgtttttccgcaaggagcaacttgcgaagccttaa

SEQ ID NO: 19 - DNA, 3463 bp, Neurospora crassa
  source 1..3463: mol_type = genomic DNA
atgccgagtgccgaatccatgaccccaagcagtgccctcggacagctcaaagcaactggacaacatgtgctatccaagcttcagcagcagacatcaaacgccgatatcatcgacatccgccgcgttgctgtagagatcaacctcaagaccgagataacctccatgttccgacctaaagatggccctagacagctacccaccttgcttctctacaacgagagaggcctgcagctgttcgagcgtgtaagtttatccaagttgtgggactgacatgctggttcatactgacccttatcagatcacataccttgaagagtactatcttaccaatgacgagatcaaaatcctcaccaaacatgcgaccgaaatggctagcttcatcccgtcaggtgccatgatcattgagctcggaagcgggtaaacatctgacaattcgacaatggggtcctggatttcgaagagctgaccatatccccagaaatctgcgcaaagtaaaccttctattggaagccctagacaacgccggcaaggcaattgactattatgcccttgacctgtctcgggaggagctggagcgcactctcgctcaggtaccatcctacaagcacgtcaagtgccacggtcttctgggtacatatgacgatggacgtgactggctcaaggccccagagaacatcaataaacagaaatgcatcttgcacctcgggtcaagcattggtaagcttgaatacgtattgaattggtcaaagaaaatcctgacatgatcgtgtctctaggcaactttaaccgcagtgacgccgctacctttctcaagggctttacggacgtccttggacccaatgacaagatgctcattggggttgacgcttgcaatgacccggcgagggtatagtacgtgttcgacaaacatcttgggacgtcatcaggaacattgctaaccatatactagccacgcttacaacggtttgtgcttactcatgtgacatggtataagaccaccttcaagctgacaataaccaaacagacaaggttggtattactcacgagtatgtcatacccctttttctgtcgctcatggcggctcccgctttccagcccattactgatactgatgattcaggttcatcttgaatggtcttcgcaacgccaatgaaattatcggagagacggccttcatcgagggcgattggagagtcattggcgaatatgtgtatgacgaagagggcggcagacaccaggccttttacgcccccactcgcgacaccatggttatgggggagttgattaggtcacacgacaggatccagatcgaacagagcctaaagtactcgaaagaggagtcagagaggctctggagcacggcgggattggaacaagtctcggaatggacgtacggcaacgaatatggtaagttgctttcacagcttttccttgcttgaggtttccagctttttctgagactacccgtttgagccgtccggctggtgttaccacaccttccccacaccctacggaccagtcgtatgatgatatgatgttgcggtgtctcctgatgggcacagctcctacactggcacaagtttaccaatgtgtaggctggtgttgggctgcagtgttcgggtagcgcgcacgggctggggaggctcgaatatttgcgccgtcaaaaccattgacacatgctgacatccctccgtcgtactgcaggactccatctgcttgccaagtcaaggatgtctttcagtctcatcccttcggtgtacgctcgcagcgcactcccaactctggacgactgggaggccctttgggcgacatgggatgtcgtcacacgtcagatgcttccccaggaagagcttctggagaagcccatcaagctccgaaacgcctgcatcttttacctcggtcacatcccgaccttcctcgacatccagctcacaaagaccaccaagcaggctccgtcagagcccgctcacttttgcaagatcttcgagcgaggcattgatcctgatgtcgacaacccggagctgtgtcatgcgcactcggagattcctgatgaatggccgccggtggaagaaatcctgacctaccaggagacggtacggtcccggttacgcggcctctatgcgcatggcatcgcgaatattccgcggaatgtgggtcgggccatttgggttgggtttgagcacgagcttatgcatatcgagacgctgttgtacatgatgctacagagcgacaagacgctgatcccaacccatattccacggcccgactttgacaagctcgcgaggaaggcagagtccgagagggttcccaatcagtggtttaagattccggcacaggagatcaccatcggtttggatgatcctgaggatggatctgatatcaacaagcattatggctggtaagtggaccttttttccgtcaaagtcgacggtggattaacttgcaaatagggacaacgagaagcctccaaggcgcgttcaagttgctgcctttcaggctcaagggaggccgatcaccaacgaagaggtacgtttgcctagcaactattattgcacgatactaacaacaaccagtacgcgcaatatctgcttgaaaagaacatcgacaagctccctgcctcttgggcccgcctggacaacgagaacattagcaatggaacaacaaacagcgtgagcggtcaccacagcaacagaacctccaagcagcagctcccttcatctttcctcgagaagacagcagtccgcacagtctacggtctcgtgcctctcaagcacgctctcgactggcccgtgtttgcctcttacgacgaacttgccggttgcgcagcttacatgggcggccgtattcccaccttcgaagagacccggagcatttacgcttacgccgatgctctcaagaagaagaaggaagctgagagacaattgggaaggacggttccggctgttaatgcgtatgtcccttcgacttactctcccgtgtccagctcgcactaacaatatctttcccattgataaacagccacctaaccaacaacggcgtggaaatcactcccccatcctctccctcttccgagacccccgccgagtcttcctccccctccgacagcaacaccaccctcatcaccaccgaagacctcttctctgacctagacggtgccaatgtcggttttcacaactggcaccctatgcccatcacctccaaaggcaacacccttgtcgggcaaggcgagctcggcggcgtgtgggaatggacttcatcggtcctccgcaagtgggaggggttcgagccgatggagctgtaccccggctatacggcggattttttcgatgagaagcacaacattgtgctgggagggagctgggctacgcatccgaggattgcggggaggaagagctttgtgaattggtaccagaggaattatccttatgcttgggtgggggcgagagttgttagggatttgtga

SEQ ID NO: 20 - DNA, 1476 bp, Neurospora crassa
  source 1..1476: mol_type = genomic DNA
atggtcgccaccaccgtcgagctgcctctgcagcaaaaggccgacgccgcccaaactgttactggccccctcccattcggcaattccctcctcaaggaattcgtcctcgaccctgcctaccggaacctcaaccatggtctgtacaacatcccccatgcatgaccccaatgtaatcactaacgaaaagcaggctccttcggcaccatcccctccgccatccaacaaaaactccgcagttaccaaaccgccgccgaagcccgcccctgccccttcctccgctaccaaacccccgtactcctcgacgaatcccgcgccgccgtcgccaacctcctcaaagtccccgtcgaaaccgtcgtcttcgtcgccaacgccactatgggcgtcaacactgtcctgcgcaacatcgtctggtccgccgacggcaaggacgagatcctctacttcgacaccatctacggcgcctgcggcaagaccatcgactacgtcatcgaagacaagcgagggatcgtttcttctcgctgtatcccattgatctaccccgccgaagacgacgatgtcgtcgctgccttccgggacgccatcaagaagagccgcgaagaaggcaagcgaccccgtctggctgttatcgacgtcgtctcctccatgcctggcgtacggttcccgttcgaggacatcgtcaagatctgcaaagaagaagagatcatctcgtgcgtggacggcgcccaaggcatcggcatggtggacctcaagatcaccgagaccgacccggattttttgattagtaactgccacaagtggctgtttactccgcgcggatgtgccgtgttctacgtgcctgtgcgtaaccagcacttgatccgctcgacgctgcctactagccatgggttcgtgccgcaggtcgggaataggttcaacccgctggtgccggcggggaacaagtcagcgtttgttagcaactttgagtttgtgggcacggtggataactcgccgttcttttgtgttaaggacgcgatcaagtggcgcgaggaggtgctcggtggggaggagaggatcatggagtacatgactaaattggcgagggaaggtggacagaaagtggcggagattctggggacgagggtgttggagaatagcacgggaacgctgatcaggtgcgccatggtcaatattgcgttgccgttcgttgtgggagaggatcccaaggcgccggtcaagttgacggagaaggaggagaaggatgttgaagggttgtatgagattccccatgaggaggcaaacatggcgttcaagtggatgtacaacgtgctgcaggacgagtttaacacgtttgtacccatgaccttccacaggaggaggttctgggccagattgagcgcgcaggtgtatttggagatgagcgatttcgagtgggcggggaagacgttgaaggagttgtgtgagagggtggctaagggggagtacaaggagagcgcctga

SEQ ID NO: 21 - DNA, 7537 bp, Aspergillus nidulans; identical to SEQ ID NO: 18
  source 1..7537: mol_type = genomic DNA

(19) United States
(12) Patent Application Publication
(10) Pub. No.: US 2024/0401095 A9
(43) Pub. Date: Dec. 5, 2024

(54) CONVERSION OF WASTE PLASTICS TO HIGH-VALUE METABOLITES

(71) Applicants: Berl OAKLEY, Lawrence, KS (US); Travis J. WILLIAMS, Los Angeles, CA (US); Yi-Ming CHIANG, Los Angeles, CA (US); Clay C. WANG, Los Angeles, CA (US); Yuhao CHEN, Los Angeles, CA (US); Swati BIJLANI, Los Angeles, CA (US); C. Elizabeth OAKLEY, Lawrence, KS (US); Christian Anthony RABOT, Los Angeles, CA (US)

(72) Inventors: Berl OAKLEY, Lawrence, KS (US); Travis J. WILLIAMS, Los Angeles, CA (US); Yi-Ming CHIANG, Los Angeles, CA (US); Clay C. WANG, Los Angeles, CA (US); Yuhao CHEN, Los Angeles, CA (US); Swati BIJLANI, Los Angeles, CA (US); C. Elizabeth OAKLEY, Lawrence, KS (US); Christian Anthony RABOT, Los Angeles, CA (US)

(73) Assignees: UNIVERSITY OF SOUTHERN CALIFORNIA, Los Angeles, CA (US); UNIVERSITY OF KANSAS, Lawrence, KS (US)

(21) Appl. No.: 18/380,447

(22) Filed: Oct. 16, 2023

Related U.S. Application Data
(60) Provisional application No. 63/416,085, filed on Oct. 14, 2022.
(60) Provisional application No. 63/436,303, filed on Dec. 30, 2022.

(51) Int. Cl. C12P 17/16; B01J 27/24; B01J 31/02; B01J 31/22; C12N 9/10; C12N 15/52; C12P 7/26

(52) U.S. Cl. CPC C12P 17/162; B01J 27/24; B01J 31/0244; B01J 31/0247; B01J 31/2208; C12N 9/1051; C12N 15/52; C12P 7/26; C12Y 204/01183; B01J 2231/70; B01J 2531/72; B01J 2531/842; B01J 2531/845

(57) ABSTRACT

A non-human organism for upgrading intermediate oxidation products formed by catalytic degradation of alkanes or polystyrenes is provided. The non-human organism is genetically modified to convert the intermediate oxidation products to secondary metabolites, and in particular to include a positive feedback loop construction in the promotor system. A method includes steps of catalytically degrading alkanes or polystyrene in an oxidizing environment to form intermediate products with one or more catalysts and contacting the intermediate products with the non-human organism such that intermediate oxidation products are converted to secondary metabolites.

### CROSS-REFERENCE TO RELATED APPLICATIONS

This application claims the benefit of U.S. provisional application Ser. No. 63/416,085 filed Oct. 14, 2022, the disclosure of which is hereby incorporated in its entirety by reference herein.

### STATEMENT REGARDING FEDERALLY SPONSORED RESEARCH OR DEVELOPMENT

This invention was made with government support under Grant No. CHE-1856395 awarded by the National Science Foundation (NSF) and Contract No. DE-AC05-76RL01830 awarded by the U.S. Department of Energy (DOE). The Government has certain rights in the invention.

### SEQUENCE LISTING

The XML file USC0336PUSP Sequences.xml of size 72,592 created Oct. 15, 2023, filed herewith, is hereby incorporated by reference.

### TECHNICAL FIELD

In at least one aspect, the present invention is related to the conversion of waste plastics to high-value metabolites.

### BACKGROUND

Plastic production is currently accelerating at a rate faster than any other material on the planet [1, 2], and is estimated to reach a global production volume of 1.1 billion tons annually by 2040. Only 9% of plastics were recycled as of 2015 (3). Millions of tons of plastics, in the form of trillions of plastic particles [4] leak from waste management systems into the environment, posing increasing threats to the food supply and ecosystems [5]. Polyesters are frequently recycled (ca. 30% of polyethylene terephthalate (PET)), unlike polyolefins (ca. 6% of low-density polyethylene (LDPE)) [2, 3]. Due to their robust microstructures and excellent physicochemical properties, polyethylenes have been utilized to deliver countless improvements to quality of life and health. Polyethylenes will remain ubiquitous and thus it is desirable master reclamation of the value embedded in these materials at the end of their lives.

The same physicochemical properties that make plastics useful also prevent their degradation and recycling. Further exacerbating this problem are the additives that necessarily accompany any post-consumer waste stream, e.g. colorings and plasticizers. Unlike polyesters and nylons, the chemical methods known to recycle or remanufacture polyethylenes are usually harsh [6, 7, 8]. Separately, oxidant-free, catalytic approaches are emerging for polyethylene upcycling [9, 10]. While these have modest yields and require energy-intensive conditions, they avoid the potential uncontrolled reactions that can result from heating organics with O₂. Still, oxidative conditions have an important advantage of tolerance to impurities associated with post-consumer polymer waste. These concerns, particularly salts, are exacerbated in samples recovered from the oceans or recycling centers.

Chemical approaches to polyethylene degradation generate a diverse distribution of products, because there are no functional handles in their pure hydrocarbon structures to direct a from cleavage either limits the value of these products or creates a challenge of separating them. Thus, there is growing interest in employing biological systems to break down plastics. Enzymatic degradation [11, 12] and microbial cell factories [13] have both shown moderate success in the degradation of certain plastics, but these efforts focus on degradation rather than value reclamation. Routes to upcycle plastics to secondary metabolites are not known, despite the enormous value creation that such a path would enable. Robust synthetic biology approaches have not yet been effectively developed to overcome existing limitations in plastic upcycling.

Accordingly, there is a need for improved methods for converting plastics to high-value metabolites.

### SUMMARY

In at least one aspect, a non-human organism for upgrading intermediate oxidation products formed by catalytic degradation of polyethylenes, polypropylenes, or polystyrenes is provided. The non-human organism is genetically modified to the intermediate oxidation products to secondary metabolites, and in particular to include a positive feedback loop construction in the promotor system.

In another aspect, a method for upgrading intermediate oxidation products formed by catalytic degradation of alkanes or polystyrenes is provided. The method includes steps of catalytically degrading linear and/or branched alkanes or polypropylene in an oxidizing environment to form intermediate oxidation products with a catalyst system that includes one or more catalysts and contacting the intermediate oxidation products with a non-human organism. Advantageously, the non-human organism is genetically modified to convert the intermediate oxidation products to secondary metabolites.

In another aspect, a method for making a biocontrol agent is provided. The method includes steps of culturing a strain of Aspergillus flavus with benzoic acid in a culture medium and collecting spores from the strain of Aspergillus flavus therefrom.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Scheme 1: The upcycling of polyethylenes to SMs. Polyethylenes are chemically degraded using metal catalysts and pressurized oxygen to generate a distribution of diacids, which are metabolized by fungi to rapidly produce structurally diverse SMs.

FIG. 1B. The distribution of diacid products after post-consumer polyethylene waste degradation using the optimized reaction.

FIG. 2. Overview of the novel promoter system driving production of asperbenzaldehyde in strain LO10050. The constitutive promoter gpdA(p) drives expression of afoA, encoding the AfoA transcription factor. AfoA binds to the promoter regions of genes afoG, afoE, and afoC within the asperbenzaldehyde BGC, leading to their expression and subsequent asperbenzaldehyde production. AfoA also binds to the afoE promoter (afoE(p)) controlling a second copy of afoA inserted elsewhere in the genome, driving additional AfoA production. This results in a positive feedback loop that generates high levels of both AfoA and asperbenzaldehyde. Note that afoD is deleted, halting conversion of asperbenzaldehyde to downstream metabolites. Other genes responsible for conversion of further downstream products to asperfuranone, the final product of the pathway, are not shown.

FIGS. 3A and 3B.(A) Paired extracted ion chromatograms generated via HPLC-DAD-MS. Asperbenzaldehyde production in (I) GMM and (II) PMM; citreoviridin production in (III) GMM and (IV) PMM; mutilin production in (V) GMM and (VI) PMM. Intensities are normalized for metabolites in each condition (B) SM yields produced by engineered fungal strains when grown in PMM and GMM liquid media. Bars represent means±SD (n=3). *P≤0.05; **P≤0.01; ***P≤0.005.

FIG. 4. The correlation between carbon count of the methanol-esterified product and corresponding GC-MS integration factor.

FIG. 5. The distribution of diacid products after model LDPE degradation under different catalytic conditions. (Referring to entries in Table 1.3, see below).

FIGS. 6A and 6B. Paired HPLC-DAD-MS traces of (A) crude polyethylene digest and (B) polyethylene digest following pH-controlled liquid-liquid extraction. Diacid and phthalic acid peaks are annotated.

FIGS. 7A and B. Mass spectra of (A) crude polyethylene digest and (B) polyethylene digest following pH-controlled liquid-liquid extraction. Differences of m/z=14 indicate consecutive additions of methylene groups. The peak representing phthalic acid is highlighted in blue.

FIG. 8. Comparative asperbenzaldehyde mass recoveries of strains LO2955, LO8355, and LO10050 cultured in lactose minimal media (LMM). Yields were 1.27, 1.85, and 4.30 g L⁻¹ from 15 g L⁻¹ of carbon source for LO2955, LO8355, and LO10050, respectively.

FIG. 9. Standard curve of asperbenzaldehyde generated via HPLC-DAD.

FIG. 10. Standard curve of citreoviridin generated via HPLC-DAD.

FIG. 11. Standard curve of mutilin generated via HPLC-DAD-MS.

FIG. 12A. Scheme 1: Upcycling PS into structurally diverse SMs and spores of biocontrol agents. Post-consumer PS was collected and subjected to catalytic, oxidative cleavage to generate BA in high yield. This BA is then utilized as a sole carbon source by engineered strains of A. nidulans to generate the SMs ergothioneine, pleuromutilin, and mutilin. Furthermore, this BA is also used to generate high quantities of spores of the atoxigenic biocontrol agent A. flavus Af36. Importantly, current approaches to bio-logically upcycle PS deliver simple, low-value compounds; thus, methods that fully exploit fungal biosynthesis rapidly to up-cycle PS have potential to create disproportionate value in the portfolio of polymer recyclates.

FIG. 12B. Mass recoveries corresponding to various PS sources. (I) Styrofoam cold box; (II) Styrofoam plate; (III) Catalina Island waste; (IV) Coffee lid; (V) Red drink cup.

FIGS. 13A, 13B, and 13C. Strategy to enable heterologous ergothioneine production in A. nidulans. (A) Top: genetic architecture of the native afo regulon in A. nidulans. AN1029 (AfoA) encodes a TF that regulates expression of each gene in the BGC, leading to the production of asperfuranone, the final product of the pathway. Bottom: replacement of the coding regions of various genes in the afo regulon with endogenous (AN7620 and AN6227 from A. nidulans) and exogenous (Afu2g15650 and Afu2g13295 from A. fumigatus, NCU04343 and NCU11365 from N. crassa) ergothioneine biosynthetic genes. Expression of AfoA is driven to high levels by alcA(p), which then binds to the native promoter regions of genes within the afo regulon, leading to their expression. The egt1 and egt2 genes from A. nidulans, A. fumigatus, and N. crassa are shown in gold, green, and purple, respectively; (B) the biosynthetic pathway of ergothioneine in N. crassa; (C) relative ergothioneine yields from strains YM267, YM812, YM820, and YM847. Bars represent means and error bars represent SDs. ****p≤0.0001; ***p≤0.0005.

FIGS. 14A and 14B. Strategy to enable heterologous mutilin and pleuromutilin production in A. nidulans. (A) Top: genetic architecture of the native afo regulon in A. nidulans. Middle: replacement of the coding regions of various genes in the afo regulon with exogenous mutilin biosynthetic genes. Expression of AfoA is driven to high levels by alcA(p), which then binds to the native promoter regions of genes within the afo regulon, leading to their expression. In total, five genes (orange) from C. passeckerianus were incorporated into the afo regulon to enable mutilin biosynthesis. Bottom: heterologous expression of two additional genes (gold) from C. passeckerianus into the afo regulon enables total reconstitution of the pleuromutilin biosynthetic pathway; (B) the biosynthetic pathway of mutilin and pleuromutilin. Dimethylallyl pyrophosphate (DMAPP) and isopentenyl pyrophosphate (IPP) first condense head-to-tail to form geranyl pyrophosphate (GPP). An additional IPP subunit condenses with GPP to form farnesyl pyrophosphate (FPP). Geranylgeranyl pyrophosphate synthase (Pl-ggs) condenses a final IPP subunit to form geranylgeranyl pyrophosphate (GGPP). GGPP undergoes intramolecular cyclization catalyzed by the gene product of Pl-cyc encoding a terpene cyclase. Two cytochrome p450s encoded by Pl-p450-1 and Pl-p450-2 catalyze the installation of two hydroxyl groups. The hydroxyl group bound to the cyclopentane ring is then oxidized to a ketone by the gene product of Pl-sdr, encoding a short-chain dehydrogenase/reductase, to form mutilin. An additional hydroxyl group bound to the cyclooctane ring is acetylated by the acetyltransferase encoded by Pl-atf. Finally, a cytochrome P450 encoded by Pl-p450-3 hydroxylates the primary carbon within the acetyl group to yield pleuromutilin, the final product of the pathway.^{57,59}

FIG. 15. Comparative metabolomics of engineered strains of A. nidulans when cultured in GMM vs. PSMM. 1.0×10⁷ spores of YM847, YM343, or YM283 were cultured in liquid GMM or PSMM. Relative SM levels were quantified using HPLC-DAD (for ergothioneine) or HPLC-DAD-MS (for pleuromutilin and mutilin). Bars represent means and error bars represent SDs. ns, not significant; ** p≤0.01.

FIG. 16. The generation of spores of A. flavus Af36 from PS-derived BA. A. flavus Af36 is an atoxigenic strain that lacks the ability to produce aflatoxins. It is currently used agriculturally by inoculation onto crops at various stages of their development. Following inoculation, it outcompetes toxigenic strains of A. flavus, thereby mitigating aflatoxin levels. Bars represent means and error bars represent SD. **** p<0.0001.

FIG. 17. Asperbenzaldehyde production in GMM compared to MINI supplemented with increasing concentrations of BA. 3.0×107 spores of LO10050 were inoculated into 125 mL Erlenmeyer flasks containing 30 mL of media. Each condition was cultured in triplicate. Cultures were incubated for six days at 37° C. with shaking at 180 rpm. Following incubation, 30 mL Me0H was added to each culture flask, which were then sonicated for one hour. 10 μL aliquots of extracts were then analyzed via HPLCDAD-MS. Extracted ion chromatograms corresponding to asperbenzaldehyde were measured for each condition.

FIG. 18. Standard curve of ergothioneine generated via HPLC-DAD.

FIG. 19. Standard curve of pleuromutilin generated via HPLC-DAD-MS.

FIG. 20. Standard curve of mutilin generated via HPLC-DAD-MS.

FIGS. 21A and 21B. Composition of Motor Oil.

FIGS. 22A and 21B. Motor Oil Digest products.

FIG. 23. Proton Distribution +/−Porphyrin Additive.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

The term “promoter” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect:10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

Throughout this application, where publications are referenced, the disclosures of these publications in their entireties are hereby incorporated by reference into this application to more fully describe the state of the art to which this invention pertains.

### ABBREVIATIONS

“AUG” means area under the curve.

“BGC” means biosynthetic gene clusters.

“DMAPP” means dimethylallyl pyrophosphate.

“FPP” means farnesyl pyrophosphate.

“FRP” means fiber-reinforced polymer.

“GMM” means glucose minimal media.

“GPP” means geranyl pyrophosphate.

“HDPE” means low-density polyethylene.

“IPP” means isopentenyl pyrophosphate.

“LDPE” means low-density polyethylene.

“LEL” means lower explosivity limit.

“NHPI” means N-hydroxyphthalimide.

“NO” means nitric oxide.

“PHA” means polyhydroxyalkanoate.

“PMM” means polyethylene minimal media.

“PPMM” means polypropylene minimal medium.

“PP” means polypropylene.

“PS” means polystyrene.

“PSMM” means PS-derived BA.

“SM” means secondary metabolite.

“TPP” means tetraphenylporphyrin.

“UEL” means upper explosivity limit.

The following sequences are provided with respect to the modified fungi set forth below: SEQ ID NO: 1 (afoA (AN1029) (Aspergillus nidulans)); SEQ ID NO: 2 (afoD (AN1033) (Aspergillus nidulans)); SEQ ID NO: 3 (alcR (AN8978) (Aspergillus nidulans); SEQ ID NO: 4 (ctvA (Aspergillus terreus)); SEQ ID NO: 5 (ctvB (Aspergillus terreus)); SEQ ID NO: 6 (ctvC (Aspergillus terreus); SEQ ID NO: 7 (ctvD (Aspergillus terreus)); SEQ ID NO: 8 (pl-ggs (Clitopilus passeckerianus)); SEQ ID NO: 9 (pl-cyc (Clitopilus passeckerianus)); SEQ ID NO: 10 (pl-p450-1 (Clitopilus passeckerianus)); SEQ ID NO: 11 (pl-p450-2 (Clitopilus passeckerianus)); SEQ ID NO: 12 (pl-sdr (Clitopilus passeckerianus)); SEQ ID NO: 13 (Pl-p450-3 (Clitopilus passeckerianus)); SEQ ID NO: 14 (Pl-atf (Clitopilus passeckerianus)); SEQ ID NO: 15 (AN7620 (egt1) (Aspergillus nidulans)); SEQ ID NO: 16 (AN6227 (egt2) (Aspergillus nidulans)); SEQ ID NO: 17 (Afu2g15650 (Aspergillus fumigatus)); SEQ ID NO: 18 (Afu2g13295 (Aspergillus fumigatus)); SEQ ID NO: 19 (NCU04343 (Neurospora crassa)); SEQ ID NO: 20 (NCU11365 (Neurospora crassa)) and SEQ ID NO: 21 (AN3307 (agsB) (Aspergillus nidulans)). In some refinements, nucleotide sequences having at least (in increasing order of preference) 70, 80, 90, 95, 97 or 99 percent identity to SEQ ID NOS 1-10 can be used.

In an embodiment, a non-human organism for upgrading intermediate oxidation products formed by catalytic degradation of alkanes or polystyrenes is provided. Characteristically, the non-human organism is genetically modified to convert alkanes and/or polystyrene oxidation products to secondary metabolites.

In another aspect, the alkanes are linear and/or branched alkanes. In a refinement, the alkanes are C₈₋₅₀ linear and/or branched alkanes. In a refinement, linear and/or branched alkanes, are polyethylenes or polypropylenes, or mixtures thereof. In another refinement, the linear and/or branched alkanes are provided as new or used motor oil-based materials.

In another aspect, the alkanes are polyethylene. In a refinement, the alkane is a low-density polyethylene having a weight average molecular weight from about 20,000 to 100,000 g/mol. In another refinement, the alkane is a high-density polyethylene having a weight average molecular weight from about 100,000 to 1,000,000 g/mol. In another refinement, the alkane is a linear low-density polyethylene having a weight average molecular weight from about 30,000 to 500,000 g/mol.

In another aspect, the alkanes are polypropylene. In a refinement, the alkane is a low-density polypropylene having a weight average molecular weight from about 50,000 to 500,000 g/mol.

In another aspect, the polystyrene has a weight average molecular weight from about 100,000 to 300,000 g/mol.

In a variation, the intermediate oxidation products include C₁₀₋₂₅ aldehydes, C₁₀₋₂₅ esters, C₁₀₋₂₅ compounds having ring (i.e., fused or unfused) systems, C₈₋₅₀ linear and/or branched alkanes, C₄₋₂₀ Carboxylic acids or dicarboxylic acids, or combinations thereof In a refinement, the secondary metabolites include ergothionine. In a further refinement, the secondary metabolites include asperbenzaldehyde. In another refinement, the secondary metabolites include citreoviridin and mutilin.

In a variation, the non-human organism is a fungus. In a refinement, the non-human organism is Aspergillus nidulans or Aspergillus flavus.

In a variation, the non-human organism is genetically modified by removing the afoD gene. Details of nucleotide sequences for modified (i.e., knockout) Aspergillus nidulans are found in U.S. Pat. No. 10,118,945; the entire disclosure of which is hereby incorporated by reference.

In another variation, the non-human organism is genetically modified by replacing the promoter of the afoA promoter with the alcA promoter (alcA(p)) in the nuclear genome.

In another variation, the non-human organism is genetically modified by replacing the promoter of the alcR gene with the constitutive gpdA promoter in the nuclear genome.

In another variation, the non-human organism is genetically modified by removing the afoD gene, replacing the promoter of the afoA gene with the gpdA promoter and inserting an additional copy of the afoA gene under control of the afoE promoter in the nuclear genome. This creates a positive feedback loop that generates high levels of both AfoA (protein) and asperbenzaldehyde.

In another variation, the non-human organism is genetically modified by deleting the entire sterigmatocystin biosynthetic gene cluster (genes stcA-stcW) and the emericellamide biosynthetic gene cluster (genes easA-easD) in the nuclear genome. (see, SEQ ID NO: 1 from U.S. Pat. No. 10,118,945; the entire disclosure of which is hereby incorporated by reference).

In another variation, each heterologously expressed gene in the non-human organism is placed under control of alcA(p) in the nuclear genome.

In another variation, the non-human organism is genetically modified by amplifying genes AN7620 and AN6227 using A. nidulans genomic DNA and inserting AN7620 and AN6227 replacing the coding regions of afoG and afoF, respectively and replacing the native promoter of afoA with alcA(p) to create strain YM267 and wherein maintenance of the native promoters of each of these genes allows for the protein AfoA to bind to native promoters and drive expression of the genes AN7620 and AN6227. In a refinement, the coding regions of afoE and afoD are replaced with the A. fumigatus egt1 (Afu2g15650) and egt2 (Afu2g13295) homologs, respectively, to yield strain YM812. In a further refinement, a third pair of ergothioneine biosynthetic genes are inserted into the regulon by replacing the coding regions of afoC and afoB with the N. crassa egt1 (NCU04343) and egt2 (NCU11365) genes to yield strain YM4820. In a further refinement, deleting the agsB gene encoding an α-1,3-glucan synthase is deleted to create strain YM4847.

In another embodiment, a method for upgrading intermediate oxidation products formed by catalytic degradation of degrading linear and/or branched alkanes or polystyrenes is provided. The method includes steps of catalytically degrading linear and/or branched alkanes or polypropylene in an oxidizing environment to form intermediate products with a catalyst system that includes one or more catalysts, and contacting the intermediate products with a non-human organism. Advantageously, the non-human organism is genetically modified to convert the intermediate oxidation products to secondary metabolites.

In a variation, the one or more catalysts include a transition metal-containing catalyst. The one or more catalysts may include MeReO₃ and oxides and halides of Co, Mn, Cu, and Re (e.g., Co(II), Mn(II), Cu(I), Re(VII), Re(V), and Re(III)). In a refinement, the one or more catalysts include a hydroxylated amine (e.g. N-hydroxyphthalimide). In another refinement, the one or more catalysts include NO. In another refinement, the one or more catalysts include Fe(acac)₂ or Fe(acac)₃. It should be appreciated that any combination of these catalysts can be used.

In another aspect of the method, the catalyst system includes a cocatalyst. In a refinement, the cocatalyst includes hydroxylated amines. For example, the cocatalyst can include hydroxysuccinamide (NHS) or hydroxylamine. The co-catalyst can also include N-hydroxyphthalimide (NHPI). In another refinement, the co-catalyst includes include NO. In a refinement, the linear and/or branched alkanes, are polyethylenes or polypropylenes, or mixtures thereof. In a particularly useful example, branched alkanes are provided as motor oil.

In another aspect of the method, the intermediate oxidation products include C₁₀₋₂₅ aldehydes, C₁₀₋₂₅ esters, C₁₀₋₂₅ compounds having imbedded ring systems, benzoic acid, C₄₋₂₀ carboxylic acids or dicarboxylic acids, thereof.

In another aspect of the method as set forth above, the secondary metabolites include ergothionine. In a refinement, the secondary metabolites include asperbenzaldehyde. In a refinement, the secondary metabolites include citreoviridin and mutilin.

In another aspect of the method as set forth above, the non-human organism a fungus, and in particular, a genetically modified fungus. Details of the genetically modified fungus are set forth above and in the experimental section below.

In another aspect, a method for making a biocontrol agent is provided. The method includes steps of culturing a strain of Aspergillus flavus with benzoic acid in a culture medium; and collecting spores of the strain of Aspergillus flavus therefrom. In a refinement, the strain of Aspergillus flavus is A. flavus Af36.

In another aspect, the benzoic acid is formed by catalytically degrading a polystyrene in an oxidizing environment to form intermediate products with a catalyst system that includes one or more catalysts. Details of the catalyst system including the one or more catalysts and the co-catalysts are set forth above.

In another aspect, a method for making a biocontrol agent is provided. The method includes steps of culturing B. bassiana GHA in a culture medium that includes polypropylene digestion products and collecting spores from the B. bassiana GHA therefrom. The polypropylene digestion products can be formed by catalytically degrading a polypropylene in an oxidizing environment to form intermediate products with a catalyst system that includes one or more catalysts. Details of the catalyst system including the one or more catalysts and the co-catalysts are set forth above. In a refinement, the culture medium that includes polypropylene digestion products can be formed by adding these digestion products to a MM. In a refinement, GMM, and MM can be used as controls.

Additional details of the embodiments set forth herein are found in C. Rabot et al. Conversion of Polyethylenes into Fungal Secondary Metabolites (First published: 3 Nov. 2022), Volume 62, Issue 4, Jan. 23, 2023 https://doi.org/10.1002/anie.202214609; Am. Chem. Soc. 2023, 145, 9, 5222-5230, Feb. 13, 2023, https://doi.org/10.1021/jacs.2c12285 and supplemental materials; C. Rabot et al., Polystyrene Upcycling into Fungal Natural Products and a Biocontrol Agent, J. Am. Chem. Soc. 2023, 145, 9, 5222-5230, Publication Date: Feb. 13, 2023 https://doi.org/10.1021/jacs.2c12285 and supplemental materials; and in U.S. Pat. No. 10,118,945; the entire disclosures of which are hereby incorporated by reference in their entireties.

The following examples illustrate the various embodiments of the present invention. Those skilled in the art will recognize many variations that are within the spirit of the present invention and scope of the claims.

### 1. Conversion of Polyethylenes Into Fungal Secondary Metabolites

### 1.1 Introduction

In this section, we aimed to exploit fungi, which produce products worth billions of dollars each year[14] to biologically upcycle polyethylenes. Their biosynthetic products include medically valuable secondary metabolites (SMs) including antibiotics, the cholesterol-lowering statins, immunosuppressants, and antifungals. [15] Because they have been reported to use diacids as carbon sources,[16,17] we sought to generate structurally diverse and pharmacologically active SMs directly from polyethylene-derived substrates.

It is shown here that post-consumer polyethylenes can be rapidly degraded to generate substrates that are suitable for upgrading by fungal metabolism. As a proof of principle, we demonstrate that these plastic-derived substrates can be used to produce the diverse SMs asperbenzaldehyde, citreoviridin, and mutilin in useful yields (Scheme 1). We also demonstrate robust genetic engineering strategies that permit the expression of biosynthetic gene clusters (BGCs) from many different organisms. Thus, in principle, this method expands the catalog of products to which polyethylenes can be upcycled to thousands of SMs.

### 1.2 Results and Discussion

### 1.2.1 Optimization of Polyethylene Digestion

By adapting conditions for the conversion of cyclohexane to adipic acid, [18] we were able to optimize an initial system for polymer cleavage. Using O₂ consumption and ¹H NMR integration for indicative signals as our characterization handles (by ¹H NMR), we eventually found conditions based on cobalt and manganese salts and a phthalamide-based NO source[8] that give useful oxidative cleavage results (Table 1.1). The distribution of α,ω-diacid products that are produced by the oxidative chemistry was further quantified by GCMS.

TABLE 1.1.
Screening of different catalytic systems for LDPE degradation: (a) In a
pressurized 300-mL Parr reactor, model LDPE (5 g) was reacted with molecular oxygen (16 bars) with
the presence of N-oxide catalyst (0.5 g) and metal catalyst (0.5 g of each) in acetic acid (75 mL) at
150° C. The reaction was manually terminated when no more oxygen consumption happened (b)
NHS = N-Hydroxysuccinimide; NHPI = N-Hydroxyphthalimide (c) Based on the relative NMR peak
intensity of alpha, beta and gamma protons: short (C4-C6), mid-short (C7-C8), medium (C9-C10),
mid-long (C11-C12) and long (C12+) (d) No reaction.

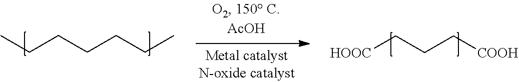

| | O₂ | | | |
|---|---|---|---|---|
| | Consumption | N-oxide | Metal | Est. Average |
| Entry^{a} | (mol) | Catalyst^{b} | Catalyst | Product Size^{c} |
| 1 | 0.110 | NH₂OH | Co(acac)₂ | Long |
| 2 | 0.093 | NHS | Co(acac)₂ | Mid-Long |
| 3 | 0.093 | TEMPO | Co(acac)₂ | Long |
| 4 | 0.223 | NHPI | Co(acac)₂ | Medium |
| 5 | 0.148 | NHPI | Mn(acac)₂ | Long |
| 6 | 0.141 | NHPI | Mn(acac)₃ | Long |
| 7 | 0.167 | NHPI | Cu(acac)₂ | Long |
| 8 | 0.167 | NHPI | Co(NO₂)₃ | Medium |
| 9 | 0.121 | NHPI | Co(NO₂)₃ + Mn(NO₂)₃ | Mid-Short |
| 10^{d} | / | NHPI | Co(acac)₂ + Mn(acac)₂ | / |
| 11 | 0.130 | TEMPO | Co(NO₂)₃ + Mn(NO₂)₃ | Long |

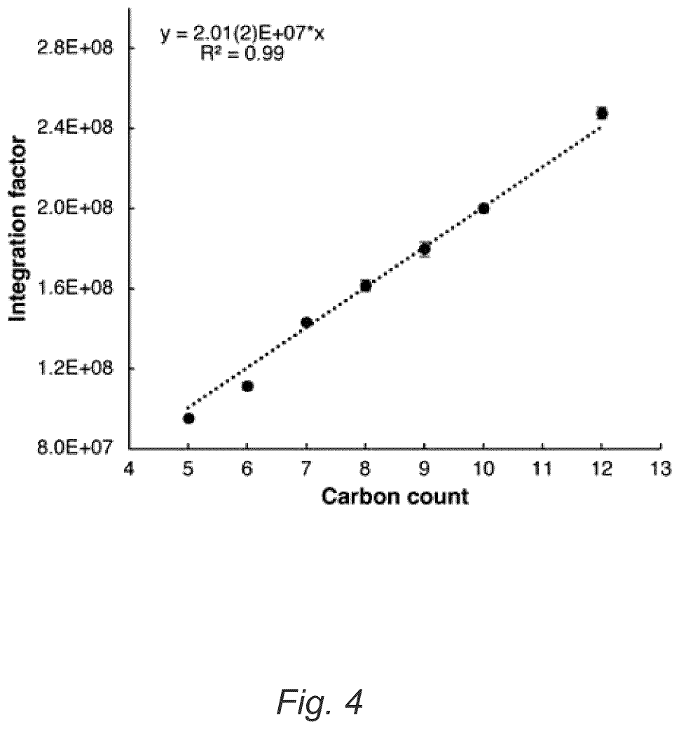

TABLE 1.2
GCMS integration factors of corresponding methanol-esterified
products: (a) Integration factors of C11, 13-18 diesters are
calculated based on the correlation curve (also see FIG. 4).

| Carbon chain | Integration Factor | | | |
|---|---|---|---|---|
| number | (vs mM) | R square | Standard Error | |
| 5 | 95082214.5 | 0.99 | 788410.3 | (0.8%) |
| 6 | 111304407.5 | 0.99 | 1899257.2 | (1.7%) |
| 7 | 143192867.3 | 0.99 | 1050684 | (0.7%) |
| 8 | 161441288.4 | 0.99 | 2807188.8 | (1.7%) |
| 9 | 179732978.1 | 0.99 | 3675148.2 | (2.0%) |
| 10 | 199947948.7 | 0.99 | 1879567.0 | (0.9%) |
| 12 | 247589859.8 | 0.99 | 2972327.8 | (1.2%) |

Prediction Curve: y = 20,090,725.4 x, S.E. = 235212 (1.2%)

| | |
|---|---|
| 11 | 220997890.5 |
| 13 | 261179431.5 |
| 14 | 281207157.0 |
| 15 | 301360882.5 |
| 16 | 321451697.0 |
| 17 | 341542333.5 |
| 18 | 361633059.0 |

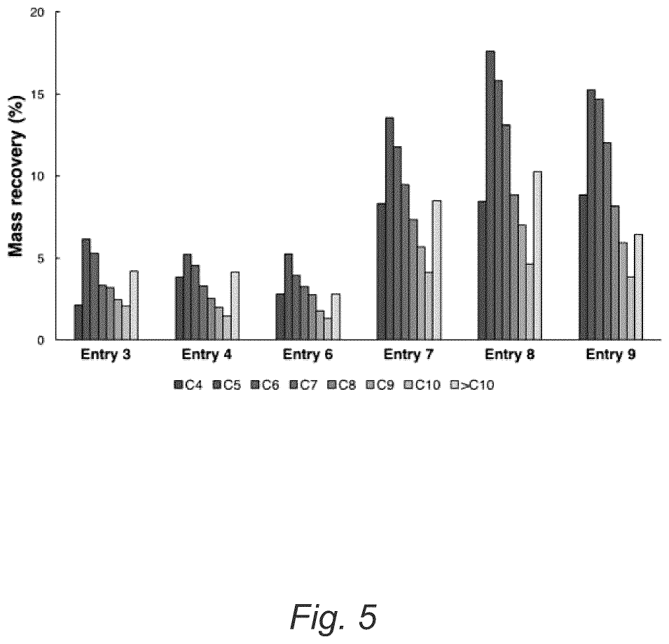

We observed that re-charging our reactor with additional O₂ did not restart the polymer cleavage reaction and hypothesized that N-hydroxyphthalimide (NHPI) serves as a source of NO, which is vented from the reactor headspace upon O₂ recharge. We see rapid hydrolysis of NHPI to phthalic acid upon reaction initiation. We further observed that our metallic catalysts lost reactivity in the recharge process (Table 1.3, entries 4-6), and that a better result was obtained when metal salts were added portion-wise along with O₂ and NHPI at recharge (compare entries 6-7). Under conditions optimized for full polymer conversion to relatively small diacid products (FIG. 5), we observed 86 wt % mass recovery (entry 8) from a 5 g sample of clean polymer. Note that branched products were not tabulated in this yield, because they could not be unambiguously identified by identity to an authentic sample. Further, addition of oxygen to the polymer adds weight, so the molar yield of carbon atoms was 52% to the named products.

TABLE 1.3A.
The optimization of reaction condition with reduced O₂ and catalysts
loading: (a) Reaction conditions unless specified mentioned otherwise: A mixture of model LDPE
(5 g), catalysts, and acetic acid (75 mL) was stirred in a 300-mL, oxygen-pressurized Parr reactor (with
active refilling) at 150° C. (b) Mass Recovery Yield (wt %) = [(Mass of diacid products)/(Mass of
starting LDPE)] × 100% (c) Molar Yield (%) = [(Mass of carbon in diacid products)/(Mass of carbon
in starting LDPE)] × 100% (d) Extra 2 wt % of each metal catalyst and 10 wt % of NHPI was added
after 6th oxygen delivery cycle (e) Additional 4 wt % of each metal catalyst and 10 wt % of NHPI were
added after 6th oxygen delivery cycle (f) Extra 4 wt % of each metal catalyst and 20 wt % of NHPI
were added after 6th oxygen delivery cycle.

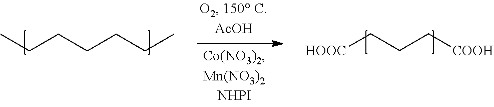

| | O₂ | O₂ | O₂ | | | Catalysts | | Mass | Molar |
|---|---|---|---|---|---|---|---|---|---|
| | Loading | Delivery | Consum. | Time | | (wt %) | | Recy. | Yield |
| Entry^{a} | (bars) | cycle | (mol) | (hrs) | Co | Mn | NHPI | (wt %)^{b} | (%)^{c} |
| 1 | 18 | 1 | 0.127 | 4 | 10 | 10 | 10 | 36.0 | 22.4 |
| 2 | 6 | 3 | 0.109 | 6.5 | 10 | 10 | 10 | 29.0 | 17.8 |
| 3 | 2 | 5 | 0.077 | 7.5 | 10 | 10 | 10 | 28.8 | 17.5 |
| 4 | 2 | 6 | 0.086 | 9.5 | 10 | 10 | 10 | 27.0 | 16.2 |
| 5 | 2 | 6 | 0.099 | 8 | 5 | 5 | 10 | 26.8 | 16.8 |
| 6 | 2 | 6 | 0.095 | 8 | 2 | 2 | 10 | 23.8 | 14.3 |
| 7^{d} | 2 | 9 | 0.150 | 10.5 | 4 | 4 | 20 | 68.8 | 41.3 |
| | cycle 1-6 | | 0.095 | 7 | 2 | 2 | 10 | | |
| | cycle 7-9 | | 0.045 | 3.5 | 2 | 2 | 10 | | |
| 8^{e} | 2 | 10 | 0.172 | 11.5 | 6 | 6 | 20 | 85.7 | 51.6 |
| | cycle 1-6 | | 0.099 | 7 | 2 | 2 | 10 | | |
| | cycle 7-10 | | 0.073 | 4.5 | 4 | 4 | 10 | | |
| 9^{f} | 2 | 12 | 0.204 | 14 | 6 | 6 | 30 | 75.1 | 44.7 |
| | cycle 1-6 | | 0.095 | 7 | 2 | 2 | 10 | | |
| | cycle 7-12 | | | | 4 | 4 | 20 | | |

TABLE 1.3b
Yield of diacid products after model LDPE degradation under
different catalytic conditions: (a) All entries are referred
to entries in Table 1.3a (b) “Heavy” portion =
a mixture of diacids comprises linear products of mass > C10.

| | Quantitative yield of diacids | | | | | | | | |
|---|---|---|---|---|---|---|---|---|---|
| | recovered from 5 grams of model LDPE | | | | | | | | |
| En- | (unit: g) | | | | | | | | |
| try^{a} | C4 | C5 | C6 | C7 | C8 | C9 | C10 | Heavy^{b} | SUM |
| 1 | 0.198 | 0.283 | 0.250 | 0.211 | 0.170 | 0.151 | 0.122 | 0.411 | 1.80 |
| 2 | 0.155 | 0.306 | 0.241 | 0.197 | 0.135 | 0.092 | 0.074 | 0.254 | 1.45 |
| 3 | 0.106 | 0.308 | 0.263 | 0.166 | 0.159 | 0.123 | 0.103 | 0.210 | 1.43 |
| 4 | 0.191 | 0.261 | 0.227 | 0.164 | 0.126 | 0.099 | 0.072 | 0.208 | 1.35 |
| 5 | 0.132 | 0.228 | 0.189 | 0.153 | 0.150 | 0.095 | 0.074 | 0.322 | 1.34 |
| 6 | 0.139 | 0.262 | 0.197 | 0.162 | 0.137 | 0.088 | 0.066 | 0.139 | 1.19 |
| 7 | 0.416 | 0.677 | 0.589 | 0.474 | 0.367 | 0.284 | 0.206 | 0.425 | 3.44 |
| 8 | 0.423 | 0.879 | 0.791 | 0.656 | 0.442 | 0.350 | 0.232 | 0.513 | 4.29 |
| 9 | 0.442 | 0.762 | 0.734 | 0.601 | 0.409 | 0.296 | 0.192 | 0.322 | 3.76 |

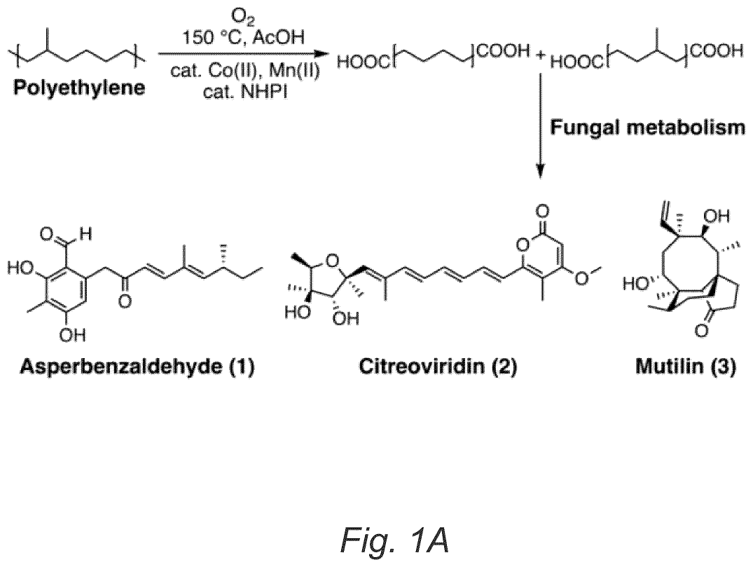

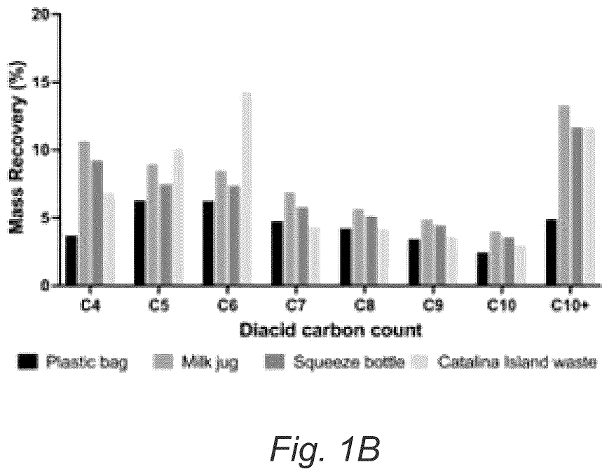

Its highly tolerant O₂-based conditions give this method the critical and distinguishing feature of tolerance of post-consumer wastes. We demonstrate that feature here with four examples (FIG. 1B & Table 1.4). Note that plasticizers and branched fragments from this LDPE film were omitted from the product accounting, although a large portion of these products are suitable for fungal digestion. A plastic grocery bag was homogenized into diacids of length C4-C12 with 34 wt % mass recovery, with an additional 2% of longer diacids (Table 1.4, entry 1). The balance of material comprises branched diacids derived from polymer branches, which are suitable for fungal metabolism. The bag must also contain colorings and plasticizers, which we account as non-products. A plastic milk jug and laboratory squeeze bottle (entries 2-3) were homogenized, respectively in 63 and 54 wt % mass recovery. These gave a distribution of products generally of higher mass than the grocery bag as shown in FIG. 1B. The higher and lower recoveries are explained by the difference of HDPE versus LDPE: the HDPE milk jug does not have polymer branches that are omitted from the recovery calculation.

TABLE 1.4a
Degradation of post-consumer PE waste using our optimized condition
(entry 8 in table 1.3): (a) Various of post-consumer PE waste
was investigated using our optimized protocol (entry 8 in table
1.3) (b) LDPE Plastic grocery tag was used (c) HDPE Milk jug
was used (d) LDPE Laboratory squeeze bottle was used (e) Plastic
waste collected from Santa Catalina Island was used.

| | | O₂ | O₂ | O₂ | Mass | Molar |
|---|---|---|---|---|---|---|
| | Time | Loading | Delivery | Consumption | Recovery | Yield |
| Entry^{a} | (hrs) | (bars) | cycle | (mol) | (wt %) | (%) |
| 1^{b} | 10.5 | 2 | 10 | 0.172 | 35.9 | 21.8 |
| 2^{c} | 11.5 | 2 | 11 | 0.199 | 62.8 | 38.5 |
| 3^{d} | 11 | 2 | 10 | 0.168 | 54.6 | 33.6 |
| 4^{e} | 10.5 | 2 | 10 | 0.182 | 58.2 | 35.5 |

TABLE 1.4b
Yield of diacid products after post-consumer PE waste
degradation using our optimized condition: (a) All
entries are referred to entries in Table 1.4a.

| | | Quantitative yield of diacids | | | | | | | |
|---|---|---|---|---|---|---|---|---|---|
| | | recovered from 5 grams of PE waste | | | | | | | |
| En- | | (unit: g) | | | | | | | |
| try^{a} | C4 | C5 | C6 | C7 | C8 | C9 | C10 | Heavy | SUM |
| 1 | 0.183 | 0.313 | 0.311 | 0.237 | 0.210 | 0.171 | 0.123 | 0.244 | 1.79 |
| 2 | 0.533 | 0.447 | 0.423 | 0.345 | 0.285 | 0.245 | 0.199 | 0.665 | 3.14 |
| 3 | 0.461 | 0.374 | 0.369 | 0.290 | 0.256 | 0.222 | 0.178 | 0.582 | 2.73 |
| 4 | 0.344 | 0.506 | 0.715 | 0.218 | 0.210 | 0.180 | 0.150 | 0.584 | 2.91 |

### 1.1.2 Fungal Upgrading of Polyethylene Digestion Products

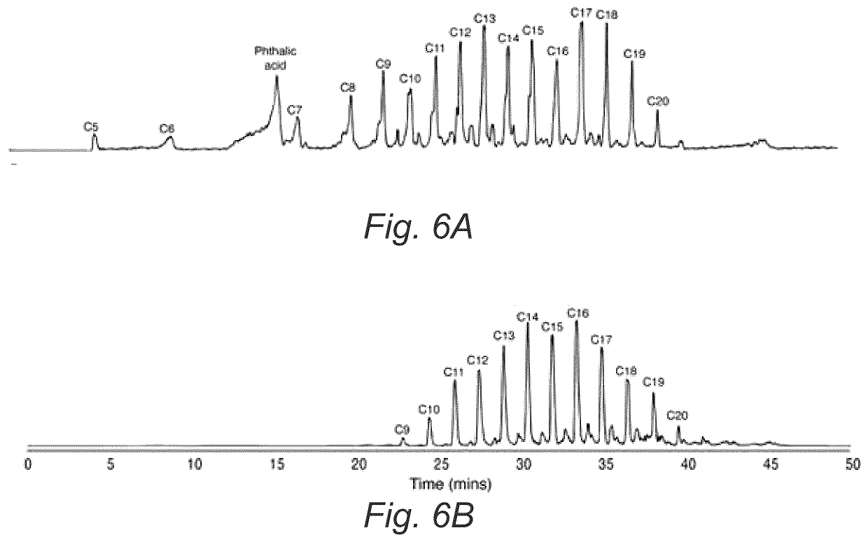

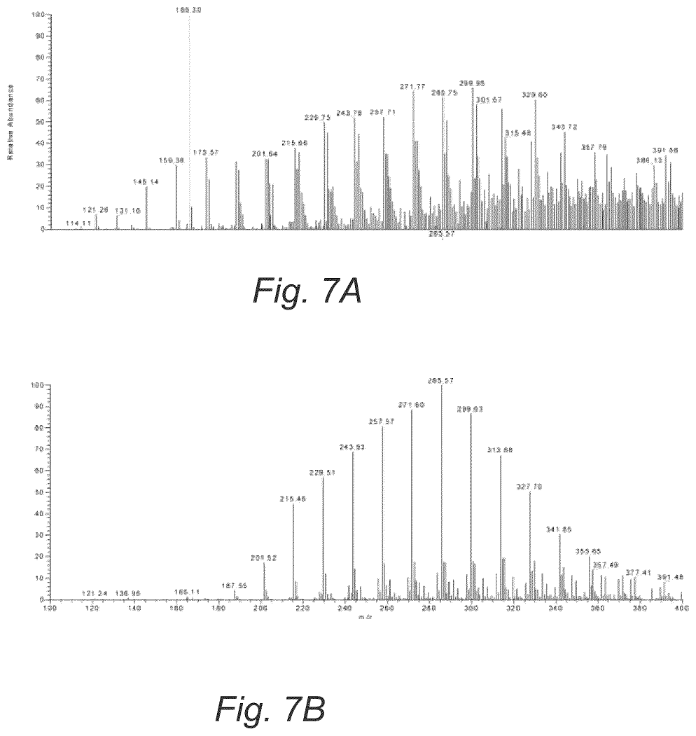

Fungi represent attractive candidates for diacid upgrading due to their robust growth capabilities, inexpensive cultivation requirements, engineerable metabolic pathways, and potential to synthesize metabolites with potent and diverse bioactivities. Short chain diacids, however, have been reported to inhibit fungal growth. [19] We confirmed that C4-C8 (photographs not shown) were toxic to the model filamentous fungus A. nidulans (strain FGSC A4) even when glucose was present as a carbon source. We found, however, that A. nidulans utilizes C10 and C12 diacids as sole carbon sources (photographs not shown) without signs of toxicity. We thus devised a system to separate polyethylene digestion products of ≥10 carbons from those <10 carbons. A series of pH-controlled liquid-liquid extractions permitted the rapid separation of C10+ diacids from light diacids and metal salts (FIGS. 6-9).

In a representative example (vide supra), 27 wt % of ocean-sourced polyethylenes were converted to diacids that were discretely identifiable using authentic standards. It should be noted that light diacids are not waste products. They may be used in large-market applications such as in the synthesis of PBCx, a biodegradable plastic emerging in agricultural applications. [20] Our data also suggest that these light diacids possess antifungal properties (photographs not shown) that may be exploited.

For attempts to produce SMs from polyethylene-derived diacids, the heavy diacid extract was added to liquid minimal media at a concentration of 10 g L−1. Liquid cultures were inoculated with fungal strains and incubated for several days (see ESI for a full extraction protocol, culture conditions, and media recipes). SMs were analyzed and quantified from culture extracts via HPLC-DAD and HPLC-DAD-MS.

Initial attempts to elicit SM production from various wild-type fungal strains resulted in only small amounts of SMs as detected via HPLC-DAD-MS. We consequently genetically engineered A. nidulans to overexpress SM biosynthetic genes or biosynthetic gene clusters (BGCs) and this proved effective, allowing robust and efficient SM production.

In order to determine the versatility of this system, we attempted to engineer fungal strains to produce various SMs using several BGC activation/expression approaches (Table 1.5). The SM used as a readout for the first of these systems was asperbenzaldehyde, a major polyketide intermediate in asperfuranone biosynthesis. [21] Asperbenzaldehyde and its derivatives disassemble tau filaments, inhibit lipoxygenases, and inhibit the interactions of the oncogenic RNA-binding proteins HuR and Musashi-1 with their target mRNAs. [22-24] We chose to target a biosynthetic intermediate because it can serve as a discovery platform that can easily be synthetically modified.

TABLE 1.5
Names, genotypes, genetic engineering techniques, and SM products of strains used in this study.

| | | Engineering | |
|---|---|---|---|
| Strain | Genotype | technique | Product |
| LO2955 | pyrG89; pyroA4, nkuAΔ::argB; riboB2, stcJΔ::AfriboB; | alcA(p) promoter | Asperbenzaldehyde |
| | afoAΔ::AfpyrG-alcA(p)-afoA; afoDΔ::AfpyroA | replacement | |
| LO8355 | pyrG89; pyroA4, nkuAΔ::argB; riboB2, stcJΔ::AfriboB; | alcA(p) promoter | Asperbenzaldehyde |
| | AfpyrG-alcA(p)-afoA, ptrA^{Res}-gpdA(p)alcR, afoDΔ::AfpyroA | replacement with | |
| | | added AlcR positive | |
| | | regulator | |
| LO10050 | pyrG89; pyroA4, nkuAΔ::argB; riboB2, afoDΔ::AfriboB, | Positive feedback loop | Asperbenzaldehyde |
| | AfpyroA-gpdA(p)-afoA, yAΔ::AtpyrG-afoE(p)-afoA, | | |
| | stcA-stcWΔ, easA-easDΔ | | |
| YM192 | pyrG89; pyroA4; nkuA::argB; riboB2; stcA-stcWΔ; | Heterologous | Citreoviridin |
| | afoA::alcA(p)-afoA; afoC-afoGΔ::ctvA-ctvB-ctvC-ctvD-AfpyrG | expression (A. ferreus | |
| | | var. aureus origin) | |
| YM283 | pyrG89, pyroA4; nkuA::argB; riboB2, stcA-stcWΔ; | Heterologous | Mutilin |
| | afoA::alcA(p)-afoA; | expression (Clitopilus | |
| | afoB-afoGΔ::pl_ggs-cyc-p450_1-p450_2-sdr-AfpyroA | passeckerianus origin) | |

We developed three strains with different systems for driving asperbenzaldehyde production: LO2955, LO8355, and LO10050. All molecular genetic modifications were executed using previously described fusion PCR-based construct generation and transformation protocols. [25] In strain LO2955, the afoD gene was deleted, blocking asperfuranone biosynthesis such that asperbenzaldehyde, its biosynthetic precursor, accumulates. Further, the promoter of the afoA gene that encodes the transcription factor (AfoA) that drives expression of the asperfuranone BGC was replaced with the alcA promoter (alcA(p)), which is highly inducible with a variety of alcohols and ketones, including methyl ethyl ketone. [26] To increase expression of AfoA, we next replaced the promoter of the alcR gene with the strong, constitutive gpdA promoter[27] in LO2955, creating strain LO8355. The alcR sequence encodes a transcription factor that drives expression of alcA. [28]

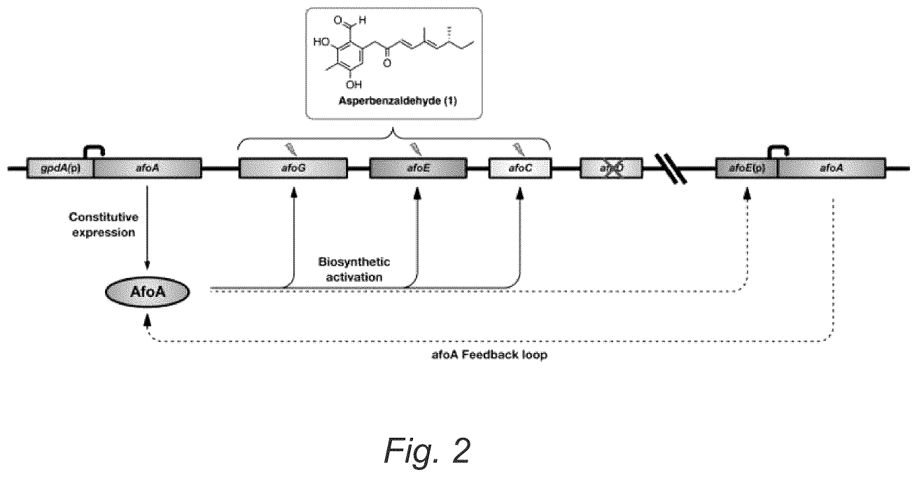

In addition, we developed a new, strong constitutive promoter system that employs a positive feedback loop (FIG. 2) and incorporated it in strain LO10050. This system requires no induction and should drive strong expression on any carbon source, whereas the AlcA system is repressed by a number of sugars including glucose. The positive feedback system is designed to drive very high levels of transcription. In addition to the new promoter system and the deletion of afoD, LO10050 also carries deletions of the entire sterigmatocystin BGC (genes AN7804-AN7825) and the emericellamide BGC (genes AN2545-AN2549). (see, SEQ ID NOS: 1 and 5 from U.S. Pat. No. 10,118,945; the entire disclosure of which is hereby incorporated by reference). Deletion of these highly expressed BGCs increases the pool of SM precursors, which are then free to feed into asperbenzaldehyde biosynthesis.

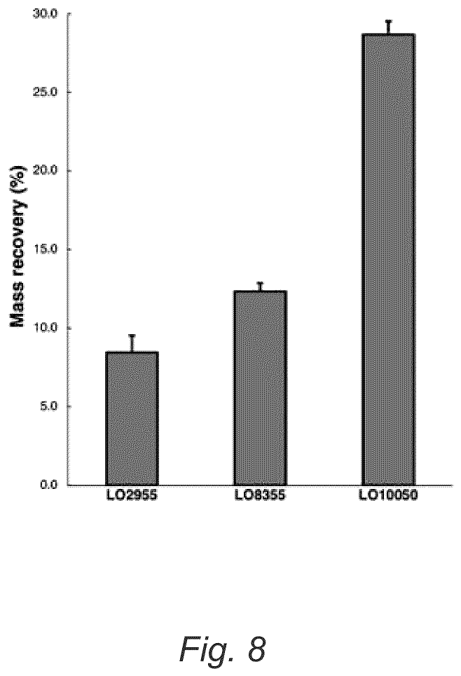

Yields of each strain grown in liquid lactose minimal media (LMM) were quantified via HPLC-DAD (FIG. 8). Each strain gave substantial yields but yields from LO10050 were the highest (4.3 g L−1 from 15 g L−1 lactose, or 29% mass conversion of lactose to asperbenzaldehyde). We therefore selected this strain to assay for asperbenzaldehyde production on polyethylene digest.

To determine the general utility of the system, we also attempted to express the diterpene antibiotic platform mutilin from the basidiomycete Clitopilus passeckerianus and the F1-ATPase β-subunit inhibitor citreoviridin from A. terreus var. aureus. [29] Mutilin is an intermediate in the biosynthetic pathway for pleuromutilin, which binds to the peptidyl transferase center of the bacterial ribosome, thus halting protein synthesis. [30] Mutilin is therefore an attractive platform for medicinal discovery efforts toward overcoming bacterial antibiotic resistance. Further, basidiomycetes are phylogenetically distant from ascomycetes such as A. nidulans and the ability to produce mutilin would indicate that this system works for BGCs from very diverse fungi. Citreoviridin is a potent mycotoxin that uncompetitively and noncompetitively inhibits ATP hydrolysis and ATP synthesis, respectively, by binding to the β-subunit of F1-ATPase. [31] Compounds in this class of mycotoxins have been investigated for the treatment of cancer. [32] In total, four genes from A. terreus var. aureus and five genes from C. passeckerianus were transferred into an A. nidulans recipient strain and placed under control of alcA(p) to generate robust producers of citreoviridin and mutilin, respectively.

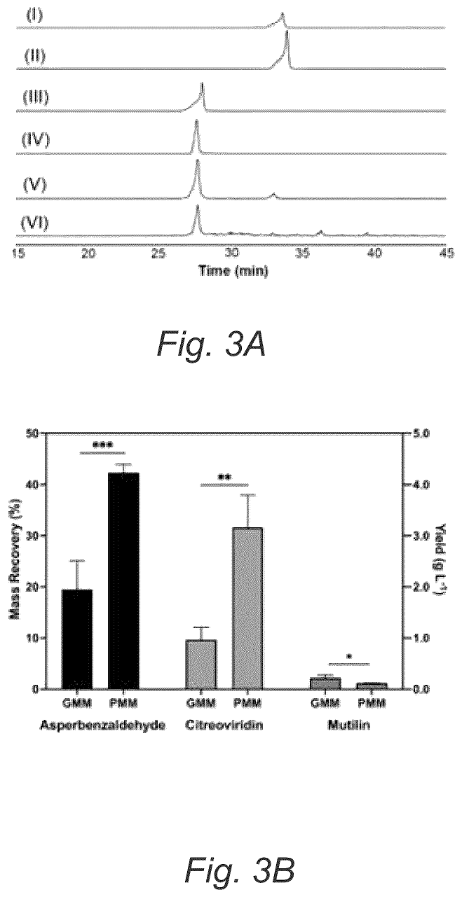

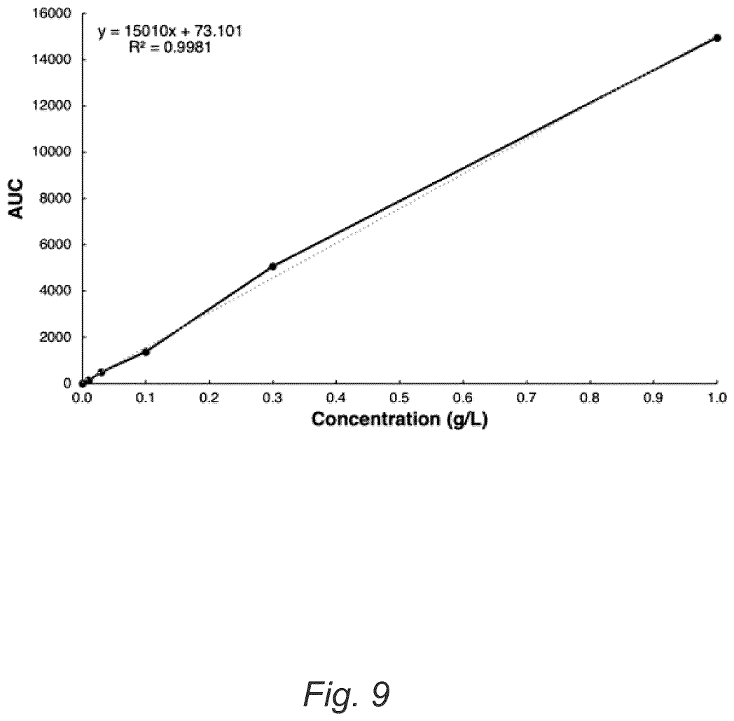

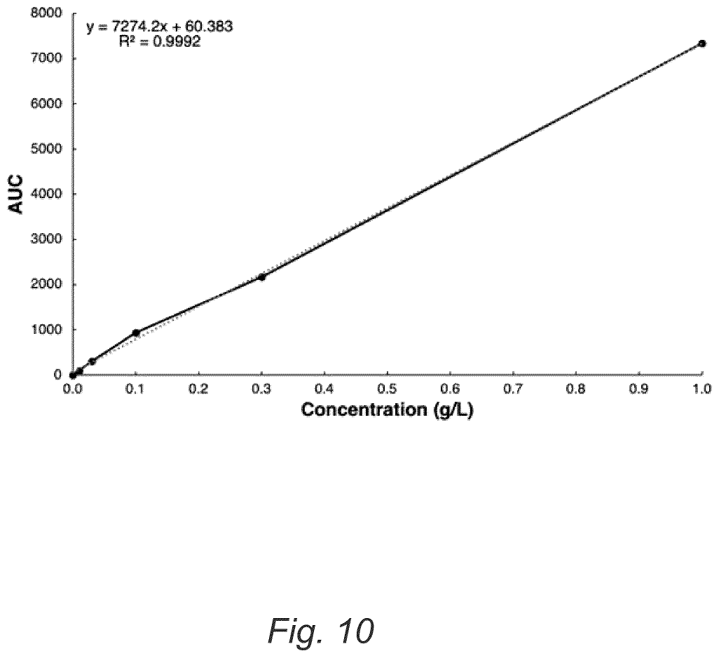

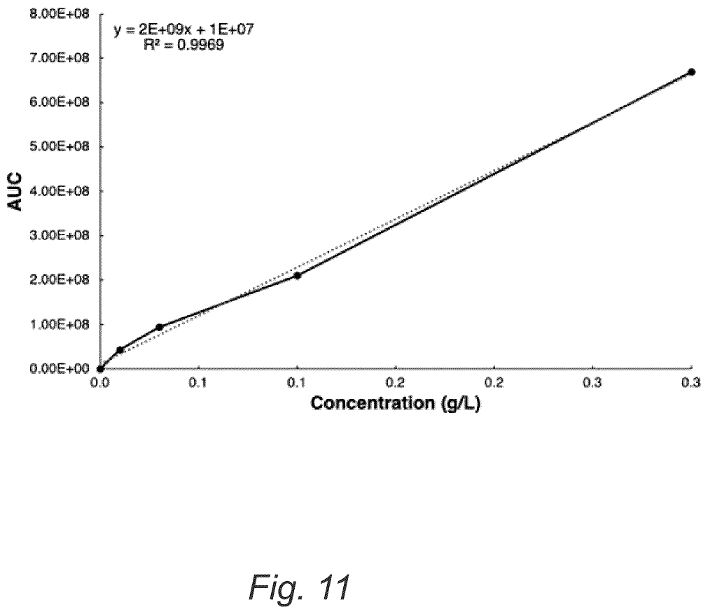

Engineered fungal strains were incubated in liquid minimal media supplemented with 10 g L⁻¹ polyethylene digest extracts (MINI, polyethylene minimal media). Culture media and/or mycelia were extracted with appropriate organic solvents, which were then analyzed via HPLC-DAD or HPLC-DAD-MS (FIG. 3). Standard curves were generated (FIGS. 9-11) using purified standards to quantify SM yields in MINI relative to glucose minimal media (GMM) or minimal media controls. SMs were purified from polyethylene digest cultures and confirmed via 1H NMR and tandem MS.

FIG. 3A provides paired extracted ion chromatograms generated via HPLC-DAD-MS. Asperbenzaldehyde production in (I) GMM and (II) PMM; citreoviridin production in (III) GMM and (IV) PMM; mutilin production in (V) GMM and (VI) PMM. In FIG. 3B, intensities are normalized for metabolites in each condition. SM yields are produced by engineered fungal strains when grown in PMM and GMM liquid media.

Our results indicate that engineered fungal strains can efficiently produce useful quantities of each target SM in under one week. Interestingly, microscopic examination of LO10050 when cultured in liquid PMM revealed initial stunted growth relative to GMM controls (by Phase contrast micrographs of asperbenzaldehyde-producing strain LO10050 in GMM and PMM, not shown). However, we observed ample hyphal growth after 48 hours and abundant asperbenzaldehyde crystals after 72 hours of incubation in PMM, which is consistent with our findings regarding asperbenzaldehyde titers.

These yields are in contrast to other metabolic engineering efforts; while high-yielding strains have been reported following extensive engineering, [33] ample SM production typically requires much larger quantities of carbon source(s) to achieve comparable yields. [34-36] It is also noteworthy that our yields were obtained from shake flasks with minimal optimization. Alteration of other culture parameters known to influence fermentation titers (e.g. culture length, media components, etc.) should permit significantly higher yields. Use of the strong constitutive promoter system may increase the production of citreoviridin and mutilin and codon optimization may further increase mutilin production.

We further note that it was not necessary to employ metabolic engineering strategies to confer the ability to metabolize polymer-derived diacids to the fungi; rather, simple extraction protocols selectively isolated diacids suitable for fungal metabolism. Finally, it is quite likely that polyethylene degradation products can be used as a carbon source in the production of other SMs. The BGCs that we have expressed are from diverse fungi and the approaches we have developed should permit the expression of BGCs from many sources. The combination of the catalytic degradation of polyethylenes with the genetic engineering of filamentous fungi represents a promising strategy for plastic upcycling.

### 1.4 Conclusion

We present a method to rapidly upcycle post-consumer polyethylenes into structurally diverse and medically useful SMs. We degrade these polyethylenes using oxidative catalysis to generate a distribution of diacids. These diacids are rapidly isolated and upgraded by engineered strains of A. nidulans to synthesize bioactive SMs. Taken together, this two-step process dramatically expands the catalog of products to which polyethylenes can be upcycled to thousands of SMs.

### 2. Polystyrene Upcycling Into Fungal Natural Products and a Bio-Control Agent

### 2.1 Catalytic PS Degradation Conditions

We initiated our investigation of conditions for PS degradation using Styrofoam insulated boxes (Table 2.1) that we shredded by hand. We monitored BA generation via 1H NMR integration as a readout for the optimization of our catalytic conditions. Low conversion efficiencies were observed with manganese(II) acetoacetonate, while the corresponding nitrate salt afforded a more reactive cleavage system (entries 1-3). Interestingly, we found that the introduction of cobalt together with manganese synergistically promoted BA generation. For example, 5 wt % each of two nitrate salts afforded a 26% yield of BA after four hours, but 10 wt % of either catalyst alone could not permit a comparable conversion (entries 3-5).

TABLE 2.1.
Screening of different catalytic conditions for PS degradation (PS source:
waste Styrofoam cold box). (a) In a 300-mL Parr reactor, a mixture of PS (5 g), metal catalyst (10
wt %), N-Hydroxyphthalimide (NHPI, 10 wt %) and acetic acid (75 mL) was stirred with molecular
oxygen (4 bars, with active refilling) at 150° C. (b) Equivalents of 02 per monomer unit in PS (c) Either
0.5 g of one catalyst or 0.25 g of two catalysts (d) Mass recovery yield (wt %) = [ (mass of benzoic
acid products)/(mass of starting PS)] x 100% (e) molar yield (%) = [(mass of carbon in
BAproducts)/(mass of carbon in starting PS)] × 100%.

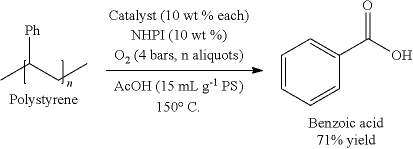

| | | O₂ | | | Mass | Molar |
|---|---|---|---|---|---|---|
| | O₂ | Consumed | Time | Metal | Recovery | Yield |
| Entry^{a} | Aliquots | Equiv^{b} | (hrs) | Catalyst^{c} | (wt %)^{d} | (%)^{e} |
| 1 | 1 | 0.75 | 4 | Mn(acac)₃ | 3.4 | 2.8 |
| 2 | 1 | 0.67 | 4 | Mn(acac)₂ | 4.5 | 3.8 |
| 3 | 1 | 0.67 | 4 | Mn(NO₃)₂ | 14.1 | 12.0 |
| 4 | 1 | 0.75 | 4 | Co(NO₃)₂ | 6.0 | 5.1 |
| 5 | 1 | 0.75 | 4 | Co(NO₃)₂+ Mn(NO₃)₂ | 30.7 | 26.2 |
| 6 | 2 | 1.50 | 8 | Co(NO₃)₂ + Mn(NO₃)₂ | 54.6 | 46.5 |
| 7 | 3 | 2.06 | 12 | Co(NO₃)₂ + Mn(NO₃)₂ | 84.1 | 71.4 |
| 8 | 1 | 0.75 | 3 | Co(NO₃)₂ + Mn(NO₃)₂ | 19.2 | 16.3 |
| 9 | 2 | 1.50 | 6 | Co(NO₃)₂ + Mn(NO₃)₂ | 47.2 | 40.1 |
| 10 | 3 | 1.98 | 9 | Co(NO₃)₂ + Mn(NO₃)₂ | 72.5 | 61.5 |

Based on these results, we hypothesize that manganese is likely to act as a Lewis acid in catalyzing electron-transfer oxidation from tertiary carbons in the polymer backbone, and cobalt tends to catalyze the β-scission for C—C bond cleavage[37]. Furthermore, under optimal conditions with portion-wise recharging of O₂, we observed the complete degradation of PS with up to 71% of starting polymer recovered as BA and 84% total mass recovery (entries 5-7). The added balance comprises incompletely digested oligomers. We also note that a reduction in reaction time did not afford increased product yield, even under optimized conditions (entries 8-10).

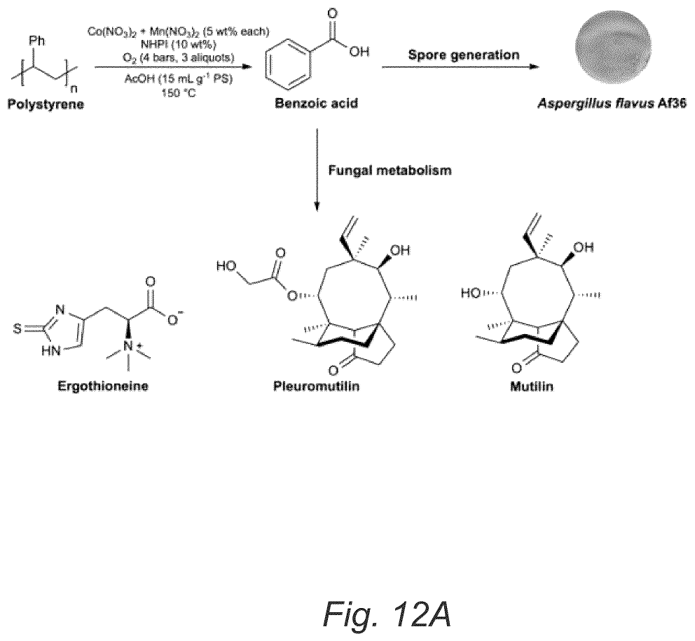

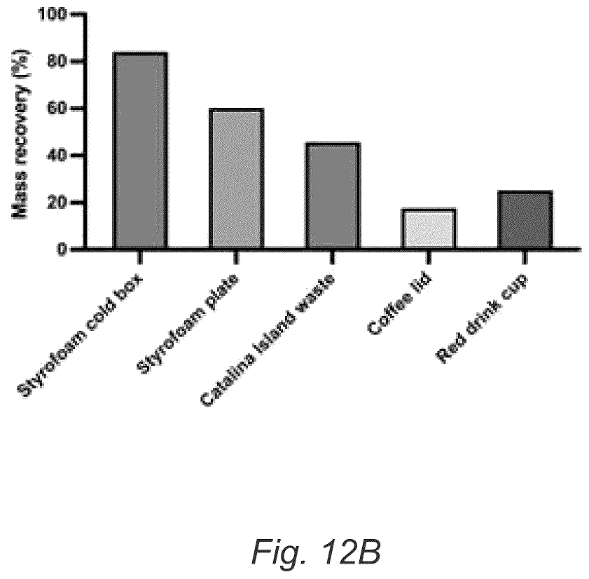

We evaluated our conditions on four additional post-consumer PS sources: a Styrofoam plate, waste collected from Santa Catalina Island, CA, a disposable coffee cup lid, and a red drink cup (FIG. 12). The Styrofoam plate and Catalina Island waste (Table 2.2, entries 2-3) were efficiently homogenized into BA, with 51 and 39% molar yield, respectively. The coffee lid and red drink cup (entries 4-5) were also degraded into BA, with 15 and 21% molar yield, respectively. The differences in recoveries can be explained by the presence of additives, such as free radical scavengers and composite polymers, which may inhibit catalytic oxidation. Trash was collected specifically from Catalina Harbor, because the harbor's unique geography accumulates pieces of the Great Pacific Garbage Patch that wash up from the North Pacific Gyre. This enables us to demonstrate our method as an approach to recycle the garbage patch itself.

TABLE 2.2
The production of benzoic acid from the degradation
of various post-consumer PS waste sources under
optimized conditions (entry 7 in table 2.1).

| | | O₂ | | | |
|---|---|---|---|---|---|
| | O₂ | Consumed | Time | Mass | Molar |
| Entry | Aliquots | Equiv. | (hrs) | Recovery (wt %) | Yield (%) |
| 1^{a} | 3 | 2.06 | 12 | 84.1 | 71.4 |
| 2^{b} | 3 | 1.80 | 12 | 60.2 | 51.1 |
| 3^{c} | 3 | 1.89 | 12 | 45.7 | 38.8 |
| 4^{d} | 2 | 1.23 | 8 | 17.6 | 15.0 |
| 5^{e} | 2 | 1.32 | 8 | 25.2 | 21.4 |

^{a}Styrofoam cold box;
^{b}Styrofoam plate;
^{c}Catalina Island waste;
^{d}Coffee lid;
^{e}Red drink cup.

Following catalysis optimization, we developed a simple procedure to isolate polymer digest for downstream fungal upgrading. A series of liquid-liquid extractions followed by recrystallization afforded BA in high purity as indicated by ¹H NMR. This isolated BA was then used for downstream metabolomics experiments. For a detailed extraction protocol, see Materials & Methods.

### 2.2 Fungal Metabolism of BA

We first confirmed that fungi can utilize BA as a sole carbon source using the model filamentous fungus A. nidulans FGSC A4 (photographs, not shown). We observed a slight discoloration in the presence of BA relative to glucose minimal media (GMM) positive controls, indicating some degree of BA-induced toxicity. We separately determined that BA was not significantly toxic to the fungus. We repeated the above experiments with phthalic acid, the hydrolysis product of the NHPI catalyst, to confirm that it is both unable to be metabolized and is nontoxic to the fungus.

For initial metabolomics experiments, we utilized the strain A. nidulans LO10050 to determine if SMs can be generated from a BA standard as a carbon source. As reported previously, [38] LO10050 has been engineered to express certain genes from the asperfuranone biosynthetic gene cluster (BGC). The incorporation of a positive feedback promoter system into this strain drives the production of the biosynthetic intermediate asperbenzaldehyde to very high levels. The high production yield of asperbenzaldehyde allowed us to easily compare initial culture conditions permissive of SM production from BA.

We took advantage of this robust asperbenzaldehyde production system to determine preliminary culture conditions for the fungal metabolism of PS-derived BA. We noticed that incubation of fungal strains in the presence of a BA standard affected the morphology of the strain; spherical mycelia characteristic of filamentous fungi cultured in shake flasks were not observed in these culture conditions. Nevertheless, a microscopic examination of the culture medium revealed the presence of extensive hyphae, revealing that growth attributable to BA was still occurring. We therefore chose to use asperbenzaldehyde production as a surrogate endpoint for fungal growth during these initial experiments.

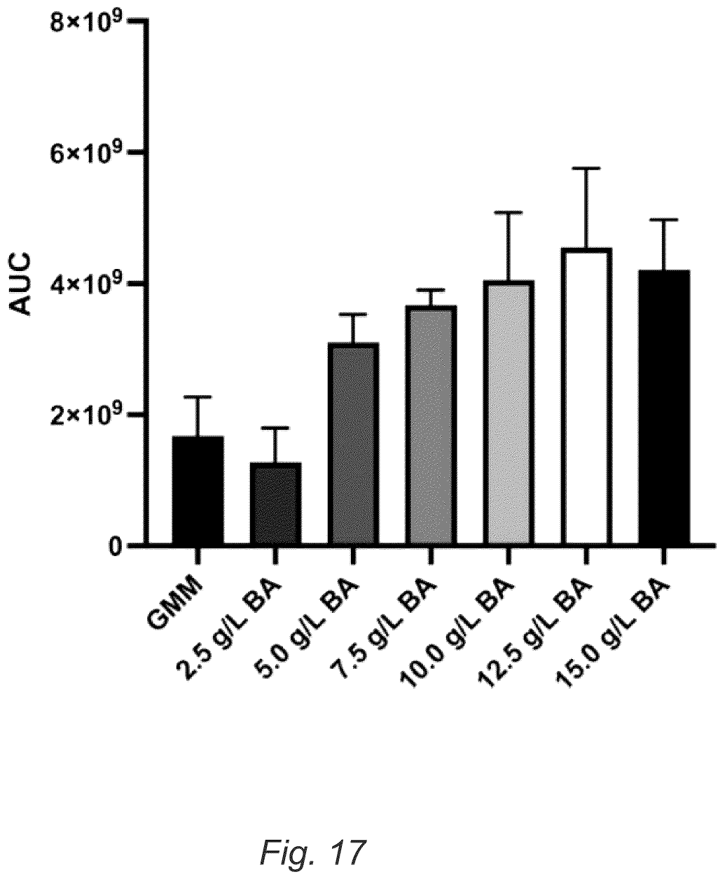

Due to its reported toxicity to fungi [39], we next determined the concentration of a BA standard that permitted the highest yield of asperbenzaldehyde in liquid cultures. We found that LO10050 could dose-dependently utilize BA as a sole carbon source to generate asperbenzaldehyde (FIG. 17): production plateaued when LO10050 was cultured in MM supplemented with 12.5 g L⁻¹ BA, with areas under the curve (AUCs) reaching ca. 270% that of GMM controls. We eventually observed a decrease in asperbenzaldehyde yield when cultures were supplemented with 15.0 g L⁻¹ BA, due to BA toxicity.

### 2.3 Strain Engineering—Ergothioneine

To promote the efficient bioconversion of waste-derived BA in high yield, several genetic engineering strategies were employed to generate three medically—and industrially relevant SMs: ergothioneine, pleuromutilin, and mutilin. The first SM that we aimed to generate from BA was ergothioneine, an unusual thio-histidine betaine amino acid. [40] Ergothioneine is a natural antioxidant that can be microbially synthesized by certain species of fungi and actinobacteria. It has been reported to exhibit anti-inflammatory and cytoprotective properties, leading to its growing application in the pharmaceutical and cosmetic industries. [41]

Although discovered more than a century ago, there has recently been exponential growth in publications related to ergothioneine. [42] The genetic basis of its biosynthesis has been elucidated both in fungi and actinobacteria. In the latter, five genes (egtABCDE) direct its biosynthesis from histidine and cysteine. [43] However, fungi can synthesize ergothioneine using only two genes: egt1 and egt2.48,49 To engineer a strain to produce ergothioneine in useful quantities, we took advantage of the robust A. nidulans afo regulon[44] to express ergothioneine biosynthetic genes. This regulon natively governs the production of the polyketide SM asperfuranone; overexpression of afoA, encoding a cluster-specific transcription factor, has been shown strongly to activate all genes within the BGC, leading to the production of very high levels of products of the cluster. To exploit the robust expression profile of this regulon, we replaced the coding regions of genes within the BGC with genes involved in ergothioneine biosynthesis.

First, BLASTp was used to identify putative A. nidulans homologs using the Neurospora crassa egt1 and egt2 as queries. This recovered two sequences bearing moderate homology to egt1 and egt2: AN7620 (63% similarity/50% identity) and AN6227 (58% similarity/43% identity). AN7620 and AN6227 were then amplified using A. nidulans genomic DNA and inserted into the coding regions of afoG and afoF, respectively. Maintenance of the native promoters of each of these genes allows for afoA to bind to them and drive their expression. We additionally replaced the native promoter of afoA with alcA(p) to create strain YM267.

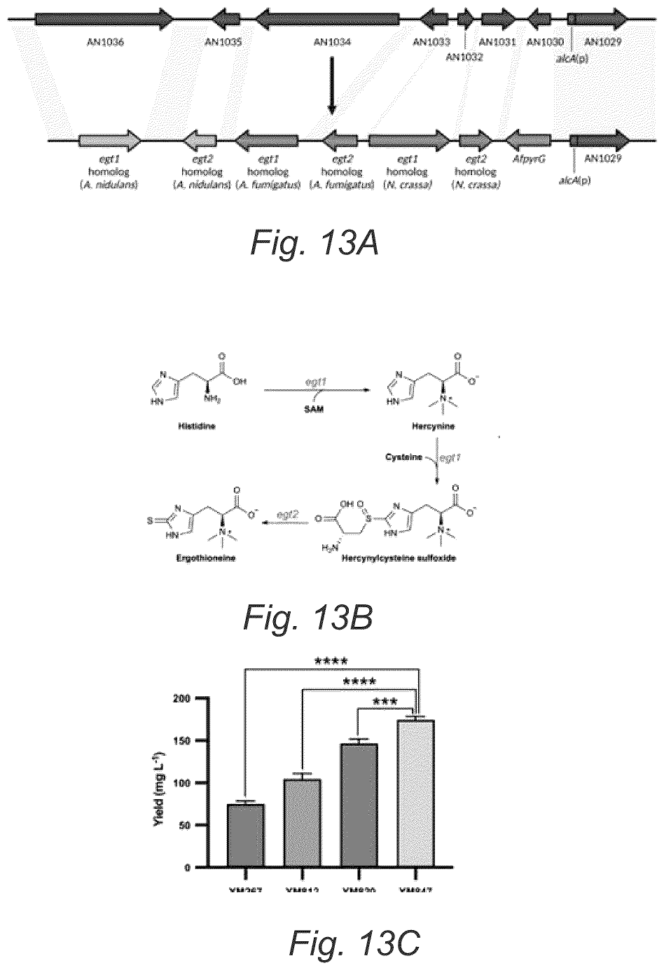

To further increase yields, we replaced the coding regions of afoE and afoD with the A. fumigatus egt1 (Afu2g15650) and egt2 (Afu2g13295) homologs, respectively, to yield strain YM812. We inserted a third pair of ergothioneine biosynthetic genes into the regulon by replacing the coding regions of afoC and afoB with the N. crassa egt1 (NCU04343) and egt2 (NCU11365) genes to yield strain YM820. Finally, we deleted the agsB gene encoding an α-1,3-glucan synthase[45] to create strain YM847. Deletion of genes encoding α-1,3-glucan synthases has been shown to improve fermentation titers by reducing hyphal clumping when grown in liquid cultures. [46] Collectively, these genetic engineering strategies enabled the generation of a strain expressing an inducible system controlling three pairs of ergothioneine biosynthetic genes (FIG. 13A). The full biosynthetic pathway for ergothioneine in N. crassa is shown in FIG. 13B. All strains described above were cultured in triplicate to determine their relative ergothioneine titers (FIG. 13C). Gratifyingly, YM847 was able to produce over 170 mg L⁻¹ ergothioneine and was therefore selected for subsequent upgrading of PS-derived BA.

### 2.4 Strain Engineering—Pleuromutilin and Mutilin

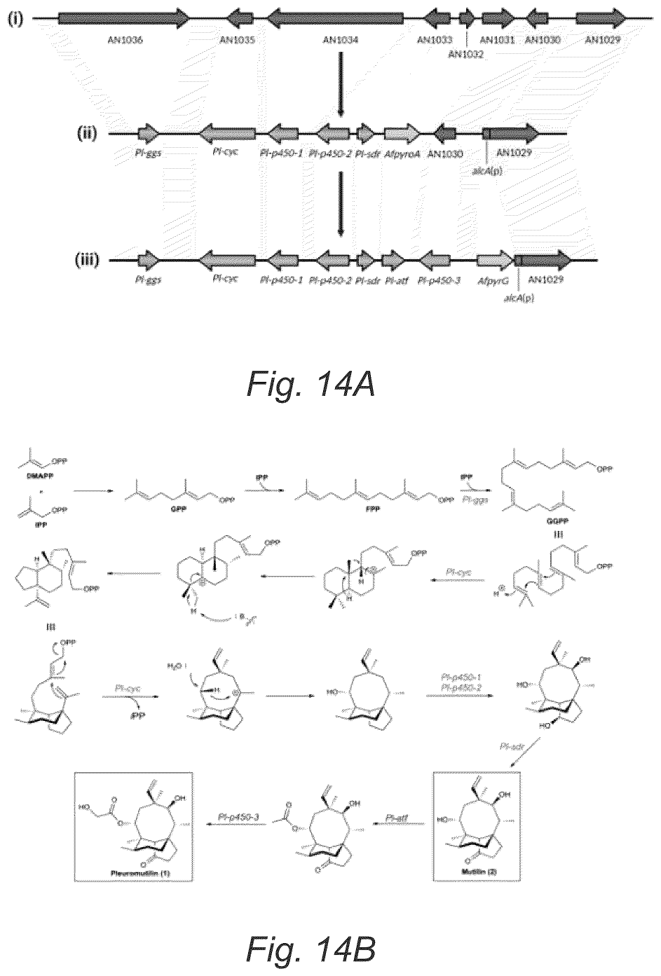

We next utilized strains of A. nidulans engineered to synthesize the SMs pleuromutilin and mutilin from BA. Pleuromutilin, a diterpene natural product produced by the basidiomycete Clitopilus passeckerianus, was first discovered in 1950[47]. It and its derivatives function by selectively inhibiting bacterial translation by binding to the peptidyl transferase center of the bacterial ribosome. [48, 49] Recently, the semisynthetic pleuromutilin derivative lefamulin was approved by the FDA for the treatment of community-acquired bacterial pneumonia. [50] Its biosynthetic pathway, involving seven genes in total, was elucidated in 2017. [51] We utilized strain YM343, reported recently by our group, [52] that was engineered to reconstitute the entire pleuromutilin biosynthetic pathway to produce pleuromutilin from PS-derived BA. Each gene of interest was placed under control of the afo regulon to drive expression to very high levels. We also utilized strain YM283, which expresses only five of the seven genes within the pleuromutilin BGC, to produce its precursor, mutilin. Production of a biosynthetic precursor of the final product in the pathway should enable late-stage synthetic derivatization. Details regarding the biosynthetic pathway of mutilin and pleuromutilin are shown in FIG. 14.

### 2.5 Comparative Metabolomics

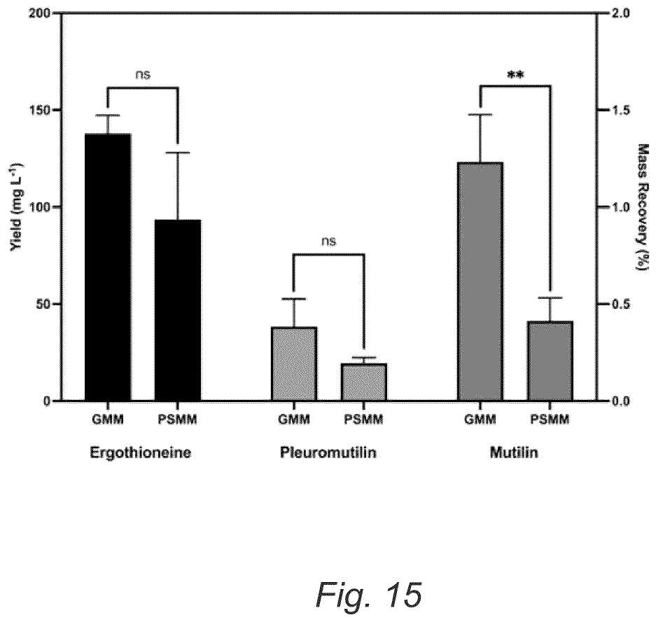

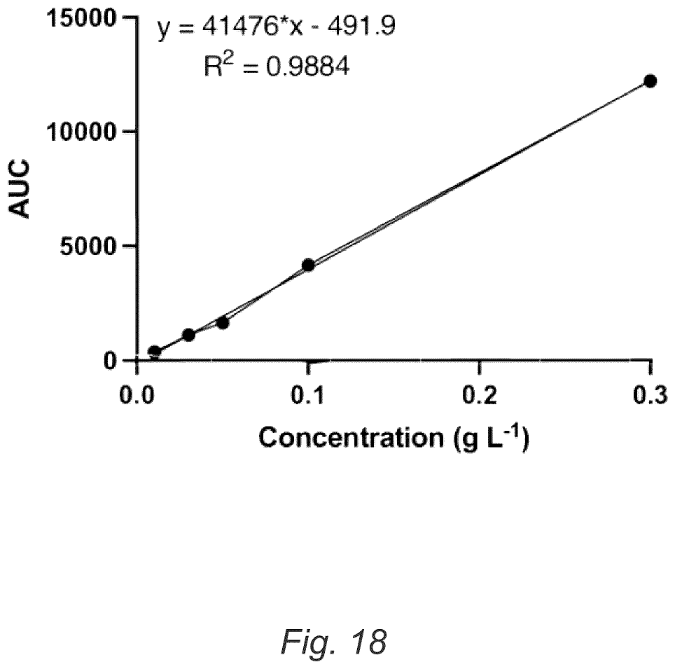

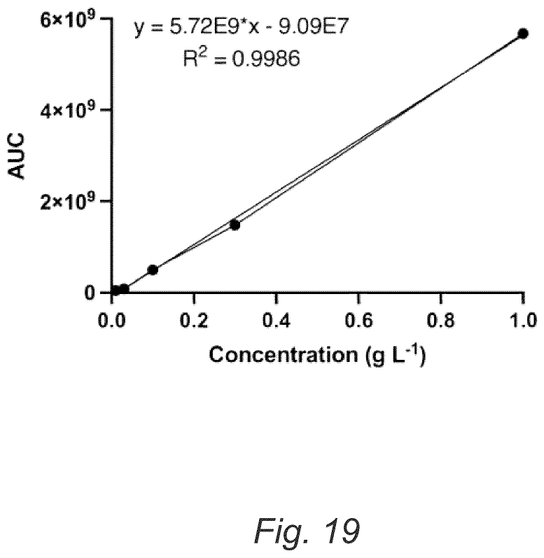

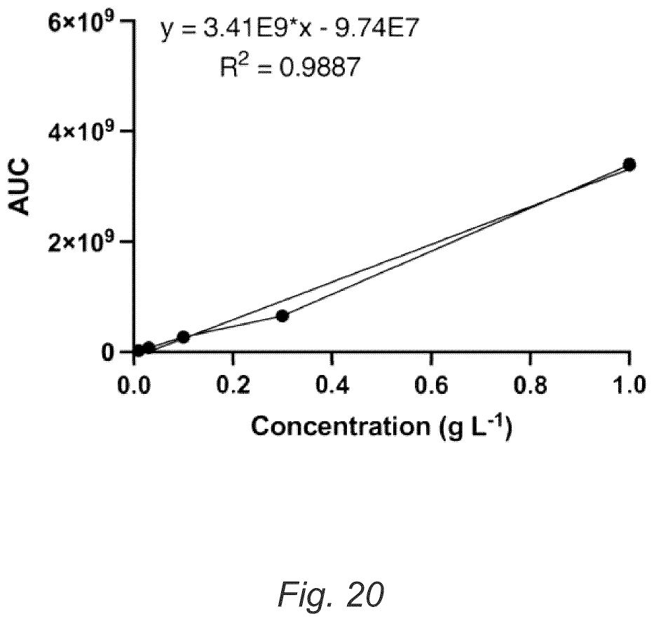

We next determined if engineered fungal strains can biosynthesize SMs from PS-derived BA. Metabolic profiling of strains YM847, YM283, and YM343 revealed that all three metabolites can be produced in useful quantities from engineered strains of A. nidulans when grown in minimal media with PS-derived BA (PSMM) as a carbon source (FIG. 15). To quantify SM yields, standard curves for each SM were generated via HPLC-DAD or HPLC-DAD-MS (FIGS. 18-20). Further, we isolated each of these SMs from large-scale cultures and confirmed their structures via 1H NMR. We note that, although SM yields in GMM are higher than in PSMM, the generation of non-trivial quantities of these valuable SMs from post-consumer PS still represents a transformative approach to PS upcycling. We further note that these strains were able to produce these SMs with minimal optimization in shake flasks. Optimization studies along with alteration of culture parameters should easily enable higher SM yields.

### 2.6 Biocontrol Agent Spore Generation

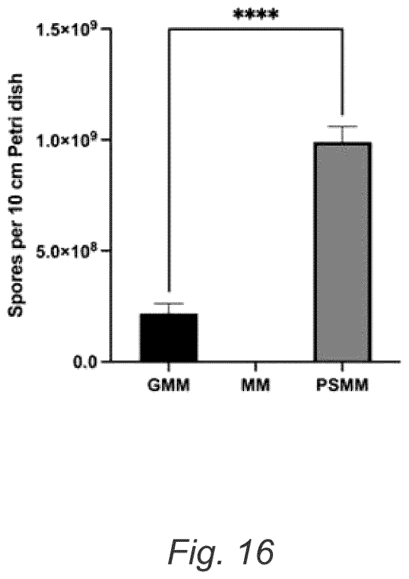

Finally, we sought to determine if spores of an agriculturally-relevant biocontrol agent can be generated from PS-derived BA. This is of particular importance to the field of plastics upcycling, because the biocatalytic products produced to date, while more valuable than the parent polymer, are not frequently used on scales that approach the quantity of plastic that will need to be reclaimed. Thus, it is valuable to add widely-used agrichemicals to our product portfolio. To determine feasibility of this application, we cultured A. flavus Af36 on solid GMM and PSMM agar plates and quantified spore generation after a seven-day incubation period. Our results indicate that spores of A. flavus Af36 can readily be generated using PS-derived BA (FIG. 16), with yields being 5.2-fold higher in PSMM relative to GMM.

### 2.7 Conclusions

Building out a robust and diversified portfolio of methods to reclaim value embedded in waste plastics, including PS, remains an evergreen challenge. Here, we couple catalytic oxidation with genetic engineering to develop a route to convert PS to fungal SMs within one week. In principle, any SM for which the biosynthetic pathway has been elucidated should be able to be generated from these PS-derived substrates, provided a robust expression system is established. We also demonstrate that we can generate spores of the agriculturally relevant biocontrol agent A. flavus Af36 from this PS-derived substrate. This adds important depth to the biocatalytic plastics upcycling portfolio because it shows a route that would consume upcycled plastics on an agrichemical scale.

Further, the enzymatic nature of biosynthesis coupled with high SM yields described here and previously[53] also implies high protein levels. Thus, this approach should also enable the production of proteins themselves from these plastic-derived substrates. Lastly, the production of other fungal fermentation products, such as organic acids, dyes, biofuels, and biopolymers should be available by these same methods. Thus, this platform effectively expands the catalog of products derived from PS from a few, structurally simple compounds to, in principle, thousands of natural products. It opens the path to apply material upcycled from ocean-sourced waste to the promotion of human health and protection of the global food supply.

### 3. A Hybrid Chemical/Biocatalysis Approach to Valorize Used Motor Oil

### 3.1 Overview

The following experiments show that (1) used motor oil can be valorized through a route analogous to the route introduced for LDPE, and that (2) ligated complexes of cobalt, manganese, iron, or copper, derived from the salts that were used on LDPE, will affect the analogous cleavage of hydrocarbon waste materials with some selectivity for larger molecular weight cleavage fragments than observed for the parent salts. This is possible because the bulk of a supporting ligand will direct oxidation by the metal to peripheral regions of a folded hydrocarbon molecule, enabling valorization of lower molecular weight waste hydrocarbons like used motor oil.

### 3.2 Chemical Valorization of Used Motor Oil

It is demonstrated that the methods described above can process motor oil into substrates that can be recycled using fungal metabolism, establishing a baseline efficiency for our method.

### 3.3. Motor Oil

The polymer cleavage chemistry set forth above can be adapted to smaller molecular weight hydrocarbon feedstocks, particularly used motor oil, which requires the development of a more end-chain-selective oxidation catalyst. To explain the problem, this section will provide background on what motor oil is and what our new chemistry must accomplish.

Engine oils and light lubricants primarily comprise C18-050 alkanes of irregular branching. [54] Some oils (American Petroleum Institute (API) Groups I-III) are natural distillates used directly. Some oils and additives comprise upgraded (usually hydrotreated) natural oils (Group IV) and synthetic poly(alpha-olefins) (Group V). Commercial engine oils also contain pH buffers, polyols, detergents, and even sulfur (1%), zinc (700 ppm), and phosphorus compounds. At the point of disposal, used engine oil contains all of these materials, plus metal particles, soot, PCBs (1 ppm), lead (16 ppm), and thermally- and oxidatively-degraded fragments from the oil itself. [55] Thus, our chemical system must tolerate all of these materials and separate or destroy any that are toxic to fungi.

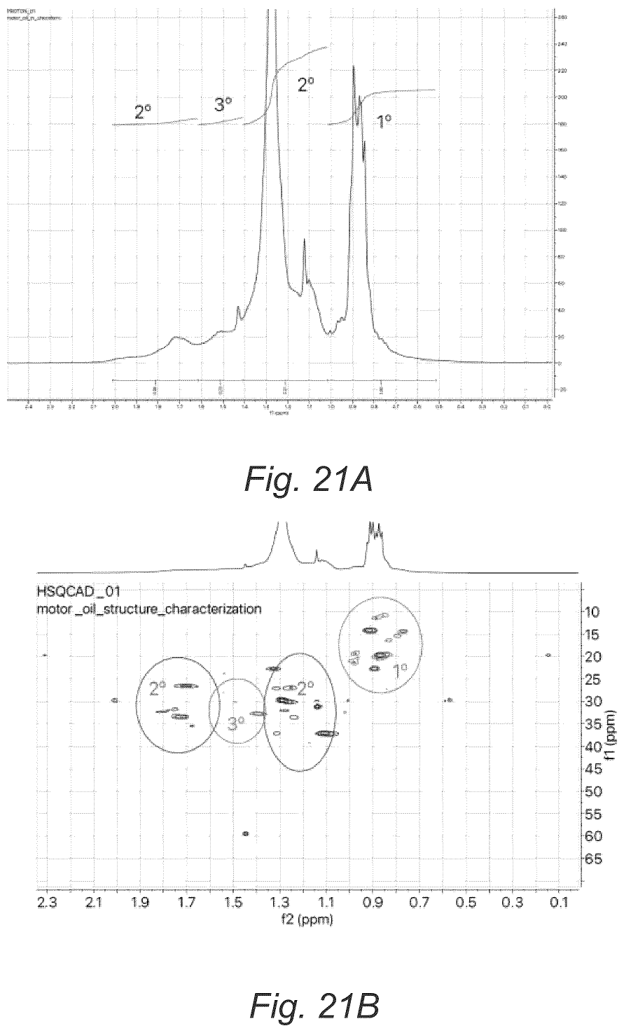

FIG. 21 shows ¹H and HSQC NMR for a semi-synthetic ASE grade 5W-30 motor oil (API Groups I-IV, blended). Some trivial (baseline) aromatic peaks are omitted, but all integrable signals are shown between 0.5 and 2.1 ppm. Phase-gated HSQC enables assignment of these as primary (methyl), secondary, or tertiary. Assuming that these are alkyl and normalizing for integration, the oil has roughly 7% methyl and 5% tertiary methine carbons with 88% secondary centers. Assuming no quaternary centers, these statistics fit a model of about 8 methylene group segments that terminate in methyl groups or methine branches. Each alkane in the oil mixture, ca 18-50 carbons, should contain 2 to 5 of these segments. It is likely that bulkier, ligated catalyst will cleave these somewhere on the peripheral chains, near the terminating methyl groups, while cleaving fewer of the internal segments.

When the polyethylene methodology was designed, it was envisioned that a linear polyethylene would be stochastically cleaved into linear diacids, and these would be recognized by fungi as if they were fatty acids. It was observed that some of our polymer digest materials are inert (will not support or inhibit growth, like unfunctionalized alkanes) or toxic (prevent growth in glucose media) to the primary host organism, Aspergillus nidulans. It was confirmed that C4-C8 (studied individually at 1/5/10 g/L)² are toxic to A. nidulans strain FGSC A4 with glucose present as a carbon source. However, A. nidulans utilizes C10 and C12 diacids as sole carbon sources without signs of toxicity.

### 3.3.1. Starting Points

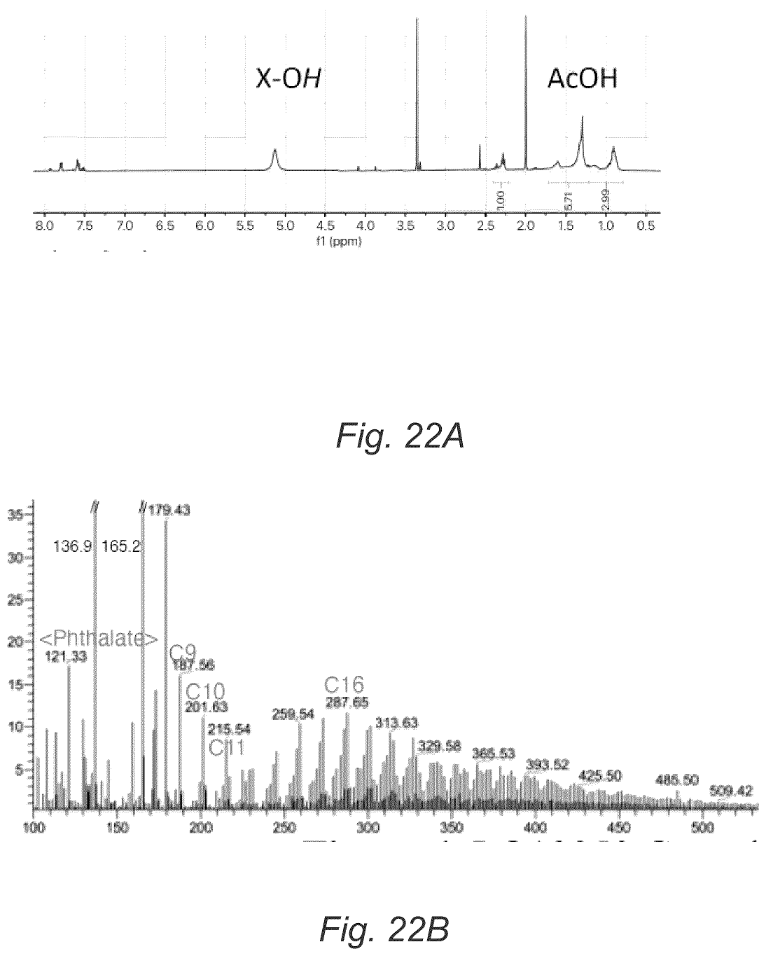

The synthetic blend (Groups I-IV) new motor oil of FIG. 21 was chemically digested with conditions optimized for the oxidation of LDPE (5 g oil, 6 bar O₂ (767 mmol consumed), 5 wt % each Co(NO₃)₂ and Mn(NO₃)₂, 10 wt % NHPI) to yield 1.8 g of acid/diacid products after extraction from pH 8 water into ethyl acetate. This is a faster reaction than those of LDPE; this is attributed to the slow reaction of insoluble, solid LDPE and a faster reaction of semi-soluble liquid oil. ¹H NMR data (500 MHz, MeOH-d₄, FIG. 22, top) for digested oil reveal a diversity of materials: the sample contains phthalic acid residual from our NHPI reagent and other aromatics. These were scantly visible in virgin oil. Signals at 2.3-2.4 are appropriate for methylene groups adjacent to carboxylic acids. Comparing these integrations to methylene and methyl clusters, respectively between 1.2-1.7 and 0.8-1.2 ppm (assigned by phase-gated HSQC, as in FIG. 21) to alpha-carboxylate methylenes (2.2-2.4) shows that there are approximately 3 CH₂ groups and 2 methyls for every carboxylate. This is consistent with methyl-terminated carboxylic acids of ca. 6 carbons and branched diacids of ca. 12 carbons. Such materials rival the minimum size appropriate for fungal upgrading but are tolerated by A. nidulans at 3 g/L (vide infra). Mass spec data are better fit to larger fragments (FIG. 22, bottom): we see large portions of C9-C11 diacid products with a broad distribution of larger materials, roughly centered around C16. These comprise a small fraction of starting oil with m/z ca. 300-700. It is expected that the balance of material includes overoxidation to small acids that were separated by extraction.

Fungi grow on motor oil digest. When our most prolific asperbenzaldehyde producer strain of A. nidulans (LO10050, vide infra) is grown on the motor oil digest of FIG. 22, we see visible fungal growth, intermediate between glucose (+) and minimal (−) media controls (photo not provided). In liquid we culture detect the formation of asperbenzaldehyde in low quantities, ca. 50 mg/L compared to ca. 1000 mg/L glucose medium, with the oil digest loaded at 3 g/L. We detect zero product with digest loaded at 10 g/L. These are critical preliminary results, because they show that (1) Fungi can grow on motor oil digest and will produce medicinal secondary metabolites from this carbon source. (2) The oil digest has significant toxicity when loaded as highly as we typically load HDPE digest. (3) The process has huge room for improvement, whereas LDPE digest will produce asperbenzaldehyde at >2× the level of glucose medium loaded at 10 g/L. While we see that there is much need to optimize the digest material that we make from motor oil, we are confident that we will find a process, and that catalyst development for selectivity should be part of the process development strategy.

### 3.3.2. Challenges of Motor Oil: Branching

The degree of branching in motor oil digest could be problematic for fungal growth, it is anticipated that this will be tolerated, as in LLDPE. The conversion of postconsumer LL/LDPE samples into fungal metabolites have been shown without observable difference in the efficiency of the fungal upgrading step compared to HDPE. We record higher yields for chemical cleavage of HDPE than LDPEs, which we attribute to branches in the polymer, not because they are not tolerated, but because we do not have analytical standards to quantify them: we pass these fragments on to fungal upgrading without quantification. Together, these observations teach us that branched fragments in LDPE are tolerated in our oxidative cleavage chemistry and that their oxidized products are substrates for fungal metabolism.

Higher branching density challenges the organism's tolerance. When PP is treated with our oxidative conditions (Table 3.1), we see hydroxylation of the polymer's tertiary centers: ¹H NMR shows the absence of carbinol protons, but alcohol carbons are present in ¹³C NMR and HMBC. We suspect that tertiary alcohols are disadvantageous for fungal metabolism. HMBC further shows a preponderance of carbonyl signals appropriate for carboxylates and none for ketones, so we are confident that we are making diacids. LC/MS and 1H DOSY data for PP digest show fragment sizes similar to PEs. We have not yet realized high yields of medicinal products from PP digest, but we have produced triacetic acid lactone, a bulk chemical, and industrially relevant biocontrol agents AF36.2 A high degree of branching reduces the effectiveness of these materials as fungus food, but they are metabolized. The level of branching in motor oil digest should be between those in LDPE and PP.

TABLE 3.1.
Conditions for PP cleavage.

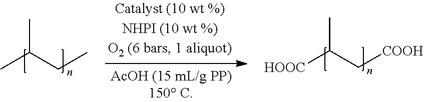

| Metal Catalyst(s) | Unreacted PP (g) |
|---|---|
| Mn(acac)₂ | 3.19 g |
| Cu(acac)₂ | 2.86 g |
| Co(acac)₂ | 2.70 g |
| Co(NO₃)₂ | 2.46 g |
| Mn(NO₃)₂ | 2.31 g |
| CO(NO₃)₂ + Mn(NO₃)₂ | 1.95 g |
| Fe(acac)₃ | Trace |

PP source: waste Falcon tube. PP waste (5 g) and catalysts were stirred in acetic acid under O₂ pressure at 150º C. for 2 hours.

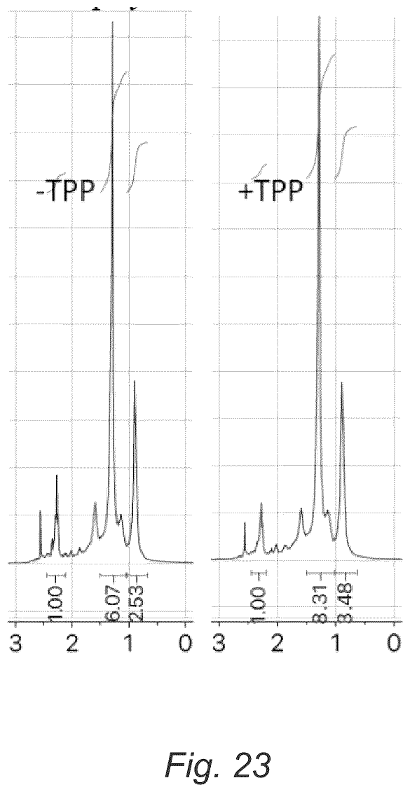

It has been shown that the addition of a bidentate nitrogen ligand (bipy, phenanthroline, TMEDA, or the Tilset MesN═C(Me)—C(Me)═NMes diamine) will halt polyethylene cleavage under our optimized conditions: bidentate N—N ligands kill the catalyst(s). Addition of tetraphenylporphyrin (TPP) does not retard reactivity in this way: in a pair of experiments, we digested 1 g motor oil using Mn(NO₃)₂+Co(NO₃)₂ (2.5 wt % each) with NHPI (10 wt %) under O₂ (6 atm, HOAc, 150° C.) and the presence and absence of TPP (10 wt %). The reactions proceed at similar conversion (per near-identical O₂ consumption), indicating that the porphyrin does not significantly stop or accelerate reactivity, possibly because it does not ligate the metal(s). Our proposal requires that the ligand should modify oxidation selectivity, which is unlikely if it does not bind a metal. We probed this through 1H NMR integration (FIG. 23): if oxidation is directed toward the periphery of the oil molecules, we should observe a higher ratio of methylene (CH₂)/methyl (CH₃) protons per each carboxylate (quantified by its alpha methylene triplet at 2.3 ppm). ¹H NMR integrations on the digest materials show ca. 35% more CH₂/CH₃ signals in the presence of TPP. This shows that the ligand can modify reaction selectivity in the desired direction without shutting off reactivity.

### 4. B. bassiana GHA Biocontrol Agents

To generate spores of B. bassiana GHA biocontrol agents, six-well cell culture plates (VWR; r=1.75 cm) containing 10 mL solid GMM, PPMNI, or MM were prepared. 1.0×10⁶ spores of B. bassiana GHA were inoculated into each well, spread using a plastic spreader, and incubated for seven days at 30° C. Following incubation, 3 mL ST solution (8.5 g L⁻¹ NaCl, 1 mL L⁻¹ Tween 80) was added to each well. A cotton swab was used to gently release spores from the agar. Spores were quantified using a haemocytometer.

Media recipes are based on minimal medium (MM), which contains 12.0 g L⁻¹ NaNO₃, 3.04 g L⁻¹ KH₂PO₄, 1.04 g L⁻¹, KCl, 1.04 g L⁻¹, MgSO₄.7H₂O, and 1 mL L⁻¹ Hutner's trace element solution (S. H. Hutner, L. Provasoli, A. Schatz, C. P. Haskins, Proc. Am. Phil. Soc. 1950, 94, 152-170; the entire disclosure of which is hereby incorporated by reference.). To create GMM and PPMNI, 3 g L⁻¹ of d-glucose or cleaned PP digest were added to MM, respectively. Culture media were adjusted to pH 8.0 with 5.5 M KOH. For solid cultures, 15 g L⁻¹ agar was added to the media. MM and GMM can be used as controls for this experiment.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

[1] U.S. Environmental Protection Agency, (EPA), “Advancing Sustainable Materials Management: 2018 Fact Sheet” (Publication 530-F-20-009, EPA, 2020; epa.gov/sites/default/files/2021-01/documents/2018_ff_fact_sheet_dec_2020_fnl_ 508.pdf).

[2] D. E. MacArthur, Beyond plastic waste. Science. 358, 843-843 (2017). 15 doi:10.1126/science.aao6749

[3] R. Geyer, J. R. Jamback, K. L. Law, Production, use, and fate of all plastics ever made. Sci.Adv. 3, e1700782 (2017). doi:10.1126/sciadv.1700782

[4] M. Eriksen, L. C. Lebreton, H. S. Carson, M. Thiel, C. J. Moore, J. C. Borerro, F. Galgani, P. G. Ryan, J. Reisser, Plastic Pollution in the World's Oceans: More than 5 Trillion Plastic20 Pieces Weighing over 250,000 Tons Afloat at Sea. PloS one. 9, e111913 (2014). doi:10.1371/journal.pone.0111913

[5] A. A. de Souza Machado, W. Kloas, C. Zarfl, S. Hempel, M. C. Rillig, Microplastics as an emerging threat to terrestrial ecosystems. Global Change Biol. 24, 1405-1416 (2018). doi:10.1111/gcb.14020

[6] L. R. Melby, Nitric Acid Oxidation of High-Density Polyethylene. Organic Chemical Aspects. Macromolecules. 5 11, 50-56 (1978). doi:10.1021/ma60061a010

[7] A. Albertsson, S. Karlsson, Environment-adaptable polymers. Polym. Degrad. Stab. 41, 345-349 (1993). doi:10.1016/0141-3910(93)90082-T

[8] A. Pifer, A. Sen, Chemical Recycling of Plastics to Useful Organic Compounds by Oxidative Degradation. Angew. Chem. Int. Ed. 37, 3306-3308 (1998). doi:10.1002/(SICI)1521-3773 (19981217)37:23<3306::AID-ANIE3306>3.0.00,2-B

[9] X. Jia, C. Qin, T. Friedberger, Z. Guan, Z. Huang, Efficient and selective degradation of polyethylenes into liquid fuels and waxes under mild conditions. Sci. Adv. 2, e1501591 (2016). doi:10.1126/sciadv.150159

[10] G. Celik, R. M. Kennedy, R. A. Hackler, M. Ferrandon, A. Tennakoon, S. Patnaik, A. M. LaPointe, S. C. Ammal, A. Heyden, F. A. Perras, M. Pruski, S. L. Scott, K. R. Poeppelmeier, A. D. Sadow, M. Delferro, Upcycling Single-Use Polyethylene into High-Quality Liquid Products. ACS Cent. Sci. 5, 1795-1803 (2019). doi:10.1021/acscentsci.9b00722

[11] F. Kawi, T. Kawabata, M. Oda, Current state and perspectives related to the polyethylene terephthalate hydrolases available for biorecycling. ACS Sustain. Chem. Eng. 8, 8894-8908 (2020). doi:10.1021/acssuschemeng.0c01638

[12] C. M. Carr, D. J. Clarke, A. Dobson, Microbial Polyethylene Terephthalate Hydrolases: Current and Future Perspectives. Front. Microbiol. 11, 571265 (2020). doi:10.3389/fmicb.2020.571265

[13] J. Ru, Y. Huo, Y. Yang, Microbial degradation and valorization of plastic wastes. Front. Microbiol. 11, 442 (2020). doi:10.3389/fmicb.2020.00442

[14] V. Meyer, M. R. Andersen, A. A. Brakhage, G. H. Braus, M. X. Caddick, T. C. Cairns, R. P. de Vries, T. Haarmann, K. Hansen, C. Hertz-Fowler, S. Krappmann, U. H. Mortensen, M. A. Pellalva, A. F. J. Ram, R. M. Head, Fung. Biol. Biotech. 2016, 3, 6.

[15] J. W. Bok, D. Hoffmeister, L. A. Maggio-Hall, R. Murillo, J. D. Glasner, N. P. Keller, Chem. & Biol. 2006, 13, 31-37.

[16] V. Côté, G. Kos, R. Mortazavi, P. A. Ariya, Sci. Total Env. 2008, 390, 530-537.

[17] D. Y. Kim, Y. H. Rhee, Appl. Microbiol. Biotechnol. 2003, 61, 300-308.

[18] Y. Ishii, K. Nakayama, M. Takeno, S. Sakaguchi, T. Iwahama, Y. Nishiyama, J. Org. Chem. 1995, 60, 3934-3935.

[19] H. Gershon, L. Shanks, Can. J. Microbiol. 1976, 8, 1198-1201.

[20] M. T. Zumsteinarno, A. Schintlmeister, T. F. Nelson, R. Baumgartner, D. Woebken, M. Wagner, H. P. Kohler, K. Mcneill, M. Sander, Sci. Adv. 2018, 4, eaas9024.

[21] Y. M. Chiang, E. Szewczyk, A. D. Davidson, N. Keller, B. R. Oakley, C. C. Wang, J. Am. Chem. Soc. 2009, 131, 2965-2970.

[22] S. R. Paranjape, Y. M. Chiang, J. F. Sanchez, R. Entwistle, C. C. C. Wang, B. R. Oakley, T. C. Gamblin, Planta Med. 2014, 80, 77-85.

[43] K. Kaur, X. Wu, J. K. Fields, D. K. Johnson, L. Lan, M. Pratt, A. D. Somoza, C. C. C. Wang, J. Karanicolas, B. R. Oakley, L. Xu, R. N. De Guzman, PLoS One 2017, 12, e0175471.

[24] L. Lan, J. Liu, M. Xing, A. R. Smith, J. Wang, X. Wu, C. Appelman, K. Li, A. Roy, R. Gowthaman, J. Karanicolas, A. D. Somoza, C. C. C. Wang, Y. Miao, R. De Guzman, B. R. Oakley, K. L. Neufeld, L. Xu, Cancers 2020, 12, 2221.

[25] E. Szewczyk, T. Nayak, C. E. Oakley, H. Edgerton, Y. Xiong, N. Taheri-Talesh, S. A. Osmani, B. R. Oakley, Nat. Protoc. 2006, 1, 3111-3120.

[26] E. H. Creaser, R. L. Porter, K. A. Britt, J. A. Pateman, C. H. Doy, Biochem. J. 1985, 225, 449-454.

[27] P. J. Punt, M. A. Dingemanse, B. J. Jacobs-Meij sing, P. H. Pouwels, C. A. van den Hondel, Gene 1988, 69, 49-57.

[28] B. Felenbok, D. Sequeval, M. Mathieu, S. Sibley, D. I. Gwynne, R. W. Davies, Gene 1988, 73, 385-396.

[29] Y. M. Chiang, T. S. Lin, S. L. Chang, G. Ahn, C. C. C. Wang, ACS Synth. Biol. 2021, 10, 173-182.

[30] S. M. Poulsen, M. Karlsson, L. B. Johansson, B. Vester, Mol. Microbio. 2001, 41, 1091-1099.

[31] E. M. Gause, M. A. Buck, M. G. Douglas, J. Biol. Chem. 1981, 256, 557-559.

[32] H. R. Park, I. J. Ryoo, S. J. Choo, J. H. Hwang, J. Y. Kim, M. R. Cha, K. Shin-Ya, I. D. Yoo, Toxicology 2007, 229, 253-261.

[33] J. Sun, H. S. Alper, J. Ind. Microbiol. Biotechnol. 2015, 42, 423-436.

[34] A. Li, N. Pfelzer, R. Zuijderwijk, P. J. Punt, BMC Biotechnol. 2012, 12, 57.

[35] A. Li, N. van Luijk, M. ter Beek, M. Caspers, P. Punt, M. van der Werf, Fung. Gen. Biol. 2011, 48, 6.

[36] S. Krull, A. Hevekerl, A. Kuenz, U. PruBe, App. Microb. Biotech. 2017, 101, 4063-4072.

[37] Minisci, F., Punta, C., & Recupero, F. (2006). Mechanisms of the aerobic oxidations catalyzed by N-hydroxyderivatives. Journal of Molecular Catalysis A: Chemical, 251(1-2), 129-149. https://doi.org/10.1016/j.molcata.2006.02.011

[38] Rabot, C., Chen, Y., Bijlani, S., Chiang, Y.-M., Oakley, C. E., Oakley, B. R., Williams, T. J., & Wang, C. C. C. (2022). Conversion of Polyethylenes into Fungal Secondary Metabolites. Angewandte Chemie International Edition, anie.202214609. https://doi.org/10.1002/anie.202214609

[39] Boschloo, J. G., Moonen, E., van Gorcom, R. F. M., Hermes, H. F. M., & Bos, C. J. (1991). Genetic analysis of Aspergillus niger mutants defective in benzoate-4-hydroxylase function. Current Genetics, 19 (4), 261-264. https://doi.org/10.1007/BF0035505244

[40] Borodina, I., Kenny, L. C., McCarthy, C. M., Parama-sivan, K., Pretorius, E., Roberts, T. J., van der Hoek, S. A., & Kell, D. B. (2020). The biology of ergothioneine, an antioxidant nutraceutical. Nutrition Research Re-views, 33 (2), 190-217.

[41] Cheah, I. K., & Halliwell, B. (2012). Ergothioneine; an-tioxidant potential, physiological function and role in disease. Biochimica et Biophysica Acta (BBA)—Molecu-lar Basis of Disease, 1822 (5), 784-793. https://doi.org/10.1016/j.bbadis.2011.09.01746

[42] Cheah, I. K., & Halliwell, B. (2021). Ergothioneine, re-cent developments. Redox Biology, 42, 101868. https://doi.org/10.1016/j.redox.2021.101868

[43] Seebeck, F. P. (2010). In Vitro Reconstitution of Myco-bacterial Ergothioneine Biosynthesis. Journal of the American Chemical Society, 132 (19), 6632-6633. https://doi.org/10.1021/ja101721e50

[44] Chiang, Y.-M., Szewczyk, E., Davidson, A. D., Keller, N., Oakley, B. R., & Wang, C. C. C. (2009). A Gene Cluster Containing Two Fungal Polyketide Synthases Encodes the Biosynthetic Pathway for a Polyketide, Asperfuranone, in Aspergillus nidulans. Journal of the American Chemical Society, 131(8), 2965-2970. https://doi.org/10.1021/ja8088185

[45] Yoshimi, A., Sano, M., Inaba, A., Kokubun, Y., Fujioka, T., Mizutani, O., Hagiwara, D., Fujikawa, T., Nishimura, M., Yano, S., Kasahara, S., Shimizu, K., Yamaguchi, M., Kawakami, K., & Abe, K. (2013). Functional Analysis of the α-1,3-Glucan Synthase Genes agsA and agsB in Aspergillus nidulans: AgsB Is the Major α-1,3-Glucan Synthase in This Fungus. PLoS ONE, 8 (1), e54893. https://doi.org/10.1371/journal.pone.0054893

[46] Miyazawa, K., Yoshimi, A., Zhang, S., Sano, M., Naka-yama, M., Gomi, K., & Abe, K. (2016). Increased enzyme production under liquid culture conditions in the industrial fungus Aspergillus oryzae by disruption of the genes encoding cell wall α-1,3-glucan synthase. Bioscience, Biotechnology, and Biochemistry, 80 (9), 1853-1863. https://doi.org/10.1080/09168451.2016.1209968

[47] Kavanagh, F., Hervey, A., & Robbins, W. J. (1951). Antibiotic Substances From Basidiomycetes. Proceedings of the National Academy of Sciences, 37 (9), 570-574. https://doi.org/10.1073/pnas.37.9.570

[48] Novak, R., & Shlaes, D. M. (2010). The pleuromutilin antibiotics: A new class for human use. Current Opinion in Investigational Drugs (London, England: 2000), 11 (2), 182-191.

[49] Poulsen, S. M., Karlsson, M., Johansson, L. B., & Vest-er, B. (2008). The pleuromutilin drugs tiamulin and valnemulin bind to the RNA at the peptidyl transferase centre on the ribosome. Molecular Microbiology, 41 (5), 1091-1099. https://doi.org/10.1046/j.1365-2958.2001.02595.x

[50] FDA approves new antibiotic to treat community-acquired bacterial pneumonia. (2019, Aug. 19).

[51] Alberti, F., Khairudin, K., Venegas, E. R., Davies, J. A., Hayes, P. M., Willis, C. L., Bailey, A. M., & Foster, G. D. (2017). Heterologous expression reveals the biosynthesis of the antibiotic pleuromutilin and generates bioactive semi-synthetic derivatives. Nature Communications, 8 (1), 1831. https://doi.org/10.1038/s41467-017-01659-1

[52] Chiang, Y.-M., Lin, T.-S., Chang, S.-L., Ahn, G., & Wang, C. C. C. (2021). An Aspergillus nidulans Plat-form for the Complete Cluster Refactoring and Total Biosynthesis of Fungal Natural Products. ACS Synthetic Biology, 10 (1), 173-182. https://doi.org/10.1021/acssynbio.0c00536

[53] Rabot, C., Chen, Y., Bijlani, S., Chiang, Y.-M., Oakley, C. E., Oakley, B. R., Williams, T. J., & Wang, C. C. C. (2022). Conversion of Polyethylenes into Fungal Sec-ondary Metabolites. Angewandte Chemie International Edition, anie.202214609. https://doi.org/10.1002/anie.202214609

[54] Ritter, S. K. What's That Stuff? Motor Oil. Chem. Eng. News 2006, 84, https://cen.acs.org/articles/84/i11/Motor-Oil.html

[55] (a) Abdalla, N.; Fehrenback, H. LCA for Regeneration of Waste Oil to Base Oil. Institut fur Energie and Umweltforschung Heidelberg (IFEU), Heidelberg, 14 Apr. 2018. https://www.ifeu.de/fileadmin/uploads/ifeu-GEIR-LCA-regeneration-waste-oil_final-version-2018corr.pdf (b) Rerefining Used Lubrication and Industrial Oils' Effects on Virgin Crude Oil Refining. California Department of Resources Recycling and Recovery. Report to the Legislature, 6 Feb. 2015. https://www2.calrecycle.ca.gov/Publications/Details/1518 (c) Geyer, R.; Kuczenski, B.; Henderson, A.; Zink, T. Life Cycle Assessment of Used Oil Management in California. California Department of Resources Recycling and Recovery. Publication # DRRR-2013-1465. 29 Jul. 2013.

[56] Gershon, H.; Shanks, L. Antifungal Properties of a,w-Alkanedicarboxylic Acids and their Dimethyl Esters. Can. J. Microbiol. 1976, 8, 1198-1201.

### Sequences

afoA (AN1029) (Aspergillus nidulans) SEQ ID NO: 1
ATGGCCGGCGAGCTCGATGAGCCACCAGCGGATATCCAGACGATGCTGG
ATGACTTTGATGTACAGGTCGCCGCGCTGAAGCAGGATGCCACGGCAACCACCACAAT
GTCGACGTCGACAGCTCTCATGCCTGCCCCAGCCATCTCATCTAAAGATGCTGCTCCTG
CTGGTGCTGGTTTATCGTGGCCTGACCCAACCTGGCTGGATCGCCAGTGGCAGGATGTC
AGCAGTACCAGCCTCGTCCCTCCATCAGACCTGACAGTCTCGTCGGCCACTACCCTAAC
CGACCCTCTCAGCTTCGACCTTTTGAACGAGACTCCTCCTCCTCCTTCTACGACGACAAC
AACGTCGACGACGAGGCGAGACTCATGTACTAAGGTCATGTTAACTGACCTCATCCGGG
CTGAATTGTACACTACCTAACTGATTTGTCTACCATGACACCTGACTGACAATGTGCAG
AGACCAACTCTACTTCGACCGGGTCCACGCCTTCTGCCCCATCATCCACCGGCGACGGT
ACTTTGCGCGGGTCGCCCGAGATAGCCATACCCCAGCACAGGCATGTCTGCAGTTCGCC
ATGCGAACGCTCGCAGCGGCAATGTCTGCTCACTGCCATCTTAGCGAGCATCTCTATGC
CGAGACCAAGGCCCTCTTGGAGACGCACAGCCAGACGCCCGCCACACCGCGAGACAAG
GTCCCGCTCGAGCACATCCAGGCCTGGCTGTTGTTAAGCCACTACGAGCTGCTGCGGAT
CGGCGTGCACCAGGCTATGCTCACGGCTGGCCGGGCCTTTCGTCTCGTGCAGATGGCAC
GACTGTCAGAGCTGGATGCCGGGTCAGATCGACAGCTCTCGCCGCCGTCTTCGTCGCCG
CCGTCTTCGCTAACCCTATCTCCTTCGGGGGAGAATGCTGAGAACTTCGTCGACGCCGA
AGAAGGCCGGCGGACGTTCTGGCTTGCTTATTGCTTTGATCGTTTGCTTTGCTTGCAGAA
TGAGTGGCCGTTAACGTTACAAGAAGAGATGGTACGTCGCGCTTCTTTTATTCTATTTAC
CTCAGAATTTATATTCAGTTATTTTTTATTCTAACCCTGCTAGATATTAACCCGCCTCCCC
TCCCTCGAACACAACTACCAGAACAATCTCCCCGCACGCACGCCCTTTCTCACTGAAGC
CATGGCCCAGACCGGGCAGAGCACAATGTCCCCGTTTGCCGAATGCATTATCATGGCCA
CCCTTCACGGCCGATGTATGACGCACCGCCGCTTCTACGCAAACAGCAACTCGACTGCG
TCCGGCTCCGAGTTCGAGTCTGGCGCCGCGACGCGAGACTTCTGTATCCGCCAGAATTG
GCTGTCGAATGCAGTGGACCGGCGAGTCCAGATGCTACAGCAGGTCTCCTCGCCCGCTG
TTGACAGCGACCCGATGCTGCTCTTCACGCAGACGCTCGGCTACCGCGCGACCATGCAC
CTGAGCGATACCGTCCAGCAAGTCTCCTGGCGGGCTCTCGCCAGCTCGCCCGTTGACCA
GCAGCTACTGAGCCCGGGCGCGACGATGTCGCTGTCGGCCGCCGCGTACCACCAGATG
GCCAGCCACGCAGCCGGCGAGATCGTCCGCCTGGCGAAGGCCGTCCCCTCGCTGAGTCC
GTTCAAGGCGCACCCGTTCCTACCCGATACGTTGGCGTGCGCCGCCACGTTCCTCTCGA
CGGGCAGTCCCGATCCCACGGGCGGCGAGGGGGTGCAGCATCTGCTACGAGTGTTAAG
CGAGCTGCGCGATACACACAGCCTGGCGCGGGATTATTTGCAGGGGTTGTCGGTGCAGA
CGCAGGACGAAGATCATAGACAGGATACGAGGTGGTATTGTACATAG
afoD (AN1033) (Aspergillus nidulans) SEQ ID NO: 2
ATGGCAGACCACGAGCAGGAACAGGAACCCCTCTCCATCGCCATCATAG
GCGGCGGCATCATCGGCCTTATGACAGCCCTGGGGCTTCTCCACCGCAACATCGGCAAG
GTGACAATCTACGAGCGCGCCTCAGCTTGGCCCGACATTGGGGCCGCATTTGCCTTCAC
CGGAATCGCCCGCGAGTGCATGCAGCGCCTTGATCCCGCAATTCTATCCGCTCTCTCCA
AAGTTGCGCAGCGCAATCCGCATGATAAAGTGCGGTACTGGGACGGATTCCACCCGAA
AAGCAAAGAGGAGGCACAGGATCCAGAGAAGAGCGTGCTGTTTGAGATCGAGGAGAA
GAATATGGCGTACTGGGCTTGCTTGAGGGGTGTTTTTCATGCGGAGATGGCGCGGTTGC
TGCCCGAGAGGGTTGTGAGGTTTGGCAAGAGGCTGGTGGCATATGAGGATGGTGGGGA
CCAGAAAGTGGTGCTGAGGTTCGAGGACGGGGAGGTGGAGGAGGCAGATATTGGTTGG
TTTTCTTCCCTGAGCTGGGTATTGGACGTTTCATAATGCTAATGCGGTGGGTGAAGTGAT
CGCATGCGATGGCGTCCACTCCACGGCCCGCAGAGTCCTCCTCGGCGCAGAACACCCTG
CTGCAAACGCCAGGTACTCTCGCAAAGCTGTCTACCGCGCTCTCGTGCCTATGCCAGCA
GCAATTGATGCGCTGGGGACCGAAAAGGCGCATGTCCAGATCGCACATTGCGGGCCGG
ACGCGCACATTGTCTCTTTTCCGGTACCCTACCTTACCTCCAGGCCTAGCATGCCGCATC
TATTCTGCAAGAAGGCGCTGATTGTGCCTAGGTAAACAACGCCCAGATCTATAATGTTT
TCCTCTTCACGCACGACTCGAACGAGTGGACTCACGGCCACACCATGACAGTGCCCAGC
TCGAAGGAGGAGATACTCTCGGCGGTCGAGAACTGGGGCCCGCATATCAAGGAACTTG
CCAGCCTCTTCCCGGAACAGCTAAGCAAGTACGCCATCTTCGATCAGGCGGACCACCCG
CTTCCTTACTATGCCGCTGGCCGGGTGGCGCTGGCCGGTGATGCCGCGCACGCGTCCAG
TCCGTTCCACGGGGCTGGGGCCTGTATGGGCGTTGAGGACGCGCTTGTTCTCGCTGAGT
TGCTTGAGAAGGTCCAGAACGGCTCCGCGTTCAAGGAGAAGAAGTCTAATATTGAGCTT
GCCCTGAAGACTTACAGCGATGTGAGGATTGAGCGTAGCCAGTGGCTTGTGAAGAGCTC
AAGGGAGATGGGAGATCTCTACGAATGGCGGTATGAAGACATTGGGGGTGACGGGGTA
AAGTGTAAGGCTGAATGGGAGAGGAGGAGCAGGGTGATTTGGGATTTCGATGTGCAGG
GCATGGTCGACCAGGCGAGAGAAGCGTATGAGCGTGCTGTAGTGAAGGTCTAG
alcR (AN8978) (Aspergillus nidulans) SEQ ID NO: 3
ATGGCAGATACGCGCCGACGCCAGAATCATAGCTGCGATCCCTGTCGCAA
GGGCAAGCGACGCTGTGATGCCCCGGTAGGTTGCCGATATCGGCTCCCCAGCGTGTGCA
CTGACAGTCGCTGAGATGTAACACAGGAAAATAGAAACGAGGCCAATGAAAACGGCTG
GGTTTCGTGTTCAAATTGCAAGCGTTGGAACAAGGATTGTACCTTCAATTGGCTCTCATC
CCAACGCTCCAAGGCAAAAGGGGCTGCACCTAGAGCGAGAACAAAGAAAGCCAGGAC
CGCAACAACCACCAGTGAACCATCAACTTCAGCTGCAACAATCCCTACACCGGAAAGT
GACAATCACGATGCGCCTCCAGTCATAAACTCTCACGACGCGCTCCCGAGCTGGACTCA
GGGGCTACTCTCCCACCCCGGCGACCTTTTCGATTTCAGCCACTCTGCTATTCCCGCAAA
TGCAGAAGATGCGGCCAACGTGCAGTCAGACGCACCTTTTCCGTGGGATCTAGCCATCC
CCGGTGATTTCAGCATGGGCCAACAGCTCGAGAAACCTCTCAGTCCGCTCAGTTTTCAA
GCAGTCCTTCTTCCGCCCCATAGCCCGAACACGGATGACCTCATTCGCGAGCTGGAAGA
GCAGACTACGGATCCGGACTCGGTTACCGATACTAATAGTGTACAACAGGTCGCTCAAG
ATGGATCGCTATGGTCTGATCGGCAGTCGCCGCTACTGCCTGAGAACAGTCTGTGCATG
GCCTCAGACAGCACAGCACGGCGATATGCCCGTTCCACAATGACGAAGAATCTGATGC
GAATCTACCACGATAGTATGGAGAATGCACTGTCCTGCTGGCTGACAGAGCACAATTGT
CCATACTCCGACCAGATCAGCTACCTGCCGCCCAAGCAGCGGGCGGAATGGGGCCCGA
ACTGGTCAAACAGGATGTGCATCCGGGTGTGCCGGCTAGATCGCGTATCTACCTCATTA
CGCGGGCGCGCCCTGAGTGCGGAAGAGGACAAAGCCGCAGCCCGAGCCCTGCATCTGG
CGATCGTAGCTTTTGCGTCGCAATGGACGCAGCATGCGCAGAGGGGGGCTGGGCTAAA
TGTTCCTGCAGACATAGCCGCCGATGAGAGGTCCATCCGGAGGAACGCCTGGAATGAA
GCACGCCATGCCTTGCAGCACACGACAGGGATTCCATCATTCCGGGTTATATTTGCGAA
TATCATCTTTTCTCTCACGCAGAGTGTGCTGGATGATGATGAGCAGCACGGTATGGGTG
CACGTCTAGACAAGCTACTCGAAAATGACGGTGCGCCCGTGTTCCTGGAAACCGCGAA
CCGTCAGCTTTATACATTCCGACATAAGTTTGCACGAATGCAACGCCGCGGTAAGGCTT
TCAACAGGCTCCCGGGAGGATCTGTCGCATCGACATTCGCCGGTATTTTCGAGACACCG
ACGCCGTCGTCTGAAAGCCCACAGCTTGACCCGGTTGTGGCCAGTGAGGAGCATCGCA
GTACATTAAGCCTTATGTTCTGGCTAGGGATCATGTTCGATACACTAAGCGCTGCAATG
TACCAGCGACCACTCGTGGTGTCAGATGAGGATAGCCAGATATCATCGGCATCTCCACC
AAGGCGCGGCGCTGAAACGCCGATCAACCTAGACTGCTGGGAGCCCCCGAGACAGGTC
CCGAGCAATCAAGAAAAGAGCGACGTATGGGGCGACCTCTTCCTCCGCACCTCGGACT
CTCTCCCAGATCACGAATCCCACACACAAATCTCTCAGCCAGCGGCTCGATGGCCCTGC
ACCTACGAACAGGCCGCCGCCGCTCTCTCCTCTGCAACGCCCGTCAAAGTCCTCCTCTA
CCGCCGCGTCACGCAGCTCCAAACCCTCCTCTATCGCGGCGCCAGCCCTGCCCGCCTTG
AAGCGGCCATCCAGAGAACGCTCTACGTTTATAATCACTGGACAGCGAAGTACCAACC
ATTTATGCAGGACTGCGTTGCTAACCACGAGCTCCTCCCTTCGCGCATCCAGTCTTGGTA
CGTCATTCTAGACGGTCACTGGCATCTAGCCGCGATGTTGCTAGCGGACGTTTTGGAGA
GCATCGACCGCGATTCGTACTCTGATATCAACCACATCGACCTTGTAACAAAGCTAAGG
CTCGATAATGCACTAGCAGTTAGTGCCCTTGCGCGCTCTTCACTCCGAGGCCAGGAGCT
GGACCCGGGCAAAGCATCTCCGATGTATCGCCATTTCCATGATTCTCTGACCGAGGTGG
CATTCCTGGTAGAACCGTGGACCGTCGTTCTTATTCACTCGTTTGCCAAAGCTGCGTATA
TCTTGCTGGACTGTTTAGATCTGGACGGCCAAGGAAATGCACTAGCGGGGTACCTGCAG
CTGCGGCAAAATTGCAACTACTGCATTCGGGCGCTGCAATTTCTGGGCAGGAAGTCGGA
TATGGCGGCGCTGGTTGCGAAGGATTTAGAGAGAGGTTTGAATGGGAAAGTTGACAGC
TTTTTGTAG
ctvA (Aspergillus terreus) SEQ ID NO: 4
ATGGCACCCATGGAGCCGATTGCCATCGTTGGCACTGCCTGCCGATTTGC
CGGCTCGTCATCCACTCCTTCCAGGCTTTGGGAACTTCTCTTAAACCCCAAGGACGTGG
CATCAGAGCCACCCGCAGATCGATTCAATATCGATGCTTTCTATGACCCGGAAGGCTCC
AACCCCATGGCGACCAATGCCCGCCAGGGGTATTTCCTTTCTGACAACGTCAAAGCCTT
CGATGCCCCGTTCTTCAATATCTCCGCAGCCGAAGCACTGGCACTCGACCCACAGCAGC
GGATGCTGCTGGAAGTCGTCTATGAATCACTGGAGACTGCTGGCCTGCGCTTAGACACT
CTCCGCGGCTCCTCGACGGGGGTCTACTGCGGTGTGATGAACTCCGACTGGGAGGGCAT
ATTCAGCGTCTCATGTGCAGCACCGCAGTATGGGAGTGTTGGGGTTGCCCGGAATAACC
TCGCTAACCGCATCTCCTACTTCTTCGACTGGCAAGGCCCGTCCATGTCCATCGATACCG
CCTGCTCAGCGAGCATGGTAGCATTGCATGATGCCGTCTCCGCACTCACTCGCCACGAC
TGCGACATGGCTGCAGCTCTAGGTGCCAACCTCATGTTGTCTCCCCAGATGTTCATCGCT
GCATCCAATTTGCAGATGTTGTCCCCAACCAGCCGCAGCCGTATGTGGGATGCGCAGGC
TGATGGTTATGCGCGTGGCGAGGGGGTCGCATCCGTGCTCTTGAAACGGCTTTCAGATG
CAGTGGCCGACGGCGACCCTATCGAATGTGTTATCCGAGCTGTCGGCGTGAACCATGAT
GGCCGTAGCATGGGTTTCACCATGCCGTCGAGTGATGCACAAGTGCAACTGATCAGGTC
TACTTATGCAAAAGCCGGATTGGATCCTCGCTGCGCGGAAGATCGACCCCAATATGTCG
AGGCCCATGGTACAGGCACGTTGGCGGGTGATCCCCAGGAAGCATCCGCCCTTCATCAG
GCCTTCTTCAGTTCCTCGGACGAGGACACTGTACTGCATGTCGGTTCCATCAAGACAGT
GGTAGGCCACGCGGAAGGGACTGCTGGTCTCGCGGGTCTCATCAAGGCATCCCTGTGCA
TTCAGCATGGCATAATACCCCCGAATCTTCTTTTCAATCGCTTGAACCCGGCTCTGGAGC
CATATGCACGGCAATTGCGAGTTCCAGTAGACGTGATCCCCTGGCCCTCCCTTCCTCCA
GGCGTTCCCCGACGTGTTTCAGTGAACTCCTTCGGCTTTGGTGGCACCAATGCTCATGTT
ATTCTGGAGAGCTATGAACCTGCTAGAGACCTCACCAAGGACGGCTTCAATCAGAATGC
GGTGCTTCCGTTTGTCTTCTCTGCGGAGTCGGATTATAGTCTTGGGTCGGTTCTGGAGCA
GTATTCCAGATATCTCTCCAGATTTTCTGACGTGGACGTACACGATCTGGCATGGACGCT
AATCGAGCGCCGTTCCGCGCTGATGCACCGTGTCGCTTTTTGGGCGCCAGATATTGCAC
ACCTCAAAAGAAGGATCCAGGATGAGGTCGCCCTCCGGAAAGCAGGGACACCCTCGAC
AGTCATCTGCCGGCCACATGGCAAGACTAGGAAGCACATTCTGGGCGTCTTCACTGGTC
AGGGTGCCCAATGGGCGCAGATGGGACTTGAACTAATCACCGCGTCCACCATTGCGCG
AGGCTGGCTGGATGAGCTGCAACAGTCTCTCGATACTTTGCCGGAGGCGTATCGTCCAG
AGTTCTCGCTCTTTCAAGAGCTTGCTGCGGATCCGGCCGCATCACGACTATCGGAGGCC
CTTCTGTCGCAGACCCTCTGCACAGCAATGCAGATTATCTGGGTGAAGGTGCTCTGGGC
TCTGAACATCCACTTGGAAGCTGTGGTCGGTCACTCATCTGGCGAGATTGCTGCGGCCT
TTGCGGCTGGCTTTCTGACAGCTGAGGATGCCATTCGCATTGCCTACCTTCGAGGTGTGT
TTTGCTCGGCTTCAGGCAGCTCGGGGGAAGGTGCGATGCTGGCCGCTGGTCTTTCGATG
GACGAAGCGACTGCACTCTGTGACGACGTATCCTCGTCTGGGGGGCGAATCAACGTGG
CAGCGTCCAACTCGCCTGAAAGCGTCACGCTCTCTGGAGACCGAGATGCAATTCTGCGA
GCTGAGCAGCAGTTGAAGGATAGGGGAGTCTTTGCCCGTCTACTTCGTGTCAGTACCGC
CTACCACTCCCATCACATGCAGCCATGTTCGCAGCCCTATCAGAACGCATTGAGTAGTT
GCAACATTCAGATTCAGGCCCCGGTGCCCACCACCACCTGGTATTCAAGCGTCTATGCT
GGGTGCCCCCTGGAGGAGCCTTCGGTCATAGAGACGCTCGGTACAGGAGAATACTGGG
CGGAAAATCTAGTCAGTCCTGTGTTGTTCTCGCAGGCACTAACGGCTGCCATATCCACC
ACAAACCCTTCCCTGGTCGTCGAAGTTGGACCTCATCCAGCTCTGAAAGGACCTGCCTT
ACAGACGATCTCAGGAATAACGTCAGGGGAGATCCCTTATATCGGGGTATCAGCCCGG
AACAATTGTGCACTTGAGTCCATAGCCACAGCCATTGGATCTTTCTGGACGCATCTTGGT
CCACAAGTCATCAATCCGCGAGGGTACCTGGCTCTTTTCCGGCCGAATGTGAGGTCTTC
AGTTGTCCGTGGGCTGCCTTTGTATCCCTTTGACCATCGCCAAGAGCACGGTTATCAGAC
CCGCAAGGCTAATGGTTGGCTGTACCGACGGTACACACCACACCCTCTGCTGGGTTCTC
TGAGTGAAGACCTCGGGGAGGGCGAGTTGCGGTGGAATCATTACCTCTCCCCCCGACG
GCTCCCATGGCTCGATGGCCACCGCGTCCAGGGCCAAATCGTGGTCCCTGCCACAGCTT
ATATCGTGATGGCTCTCGAGGCCGCTCGCATACTGACCGCTGAGAAACAAAAGAGCTTG
CATCTAATCCGTATAGACGACCTAGTCATCGGTCAAGCTATCTCCTTCCAGGATGAACG
AGATGAGGTTGAGACTCTGTTCCACCTCGCCCCTATGGTGGAGACCAAGGATGACAACA
CAGCAGTCGGCCGGTTCCGCTGTCAGATGGCTGCTTCCGGGGGTCACGTCAAGACATGT
GCGGAGGGCATCCTCACGGTAACCTGGGGCTCGCCGCTGGATGATGTCCTCCCATACCC
TAGGTCTCCAGCGCCCGCAGGGCTAGCCCATGTAGCCGACATAGACGAGTACTATGCGT
CGCTCCGAAGCTTGGGTTACGAGTACACCGGCGCCTTCCAGGGAATTTTTTCTCTCTCCC
GGAAGATGGGTATCGCCACGGGCCAATTGTGTAACCCTGCATTAAATGGCTTTCTGATC
CATCCAGCAGTTCTCGACACTGGATTACAGGGTCTTCTGGCCGCGGTGGGGGAGGGACA
CCTCACGAGCCTACATGTTCCAACCCGCATTGATGCATTCAGCGTAAACCCTGCAGCCT
GTAGTAGCGGTTCGCTAGCCTTTGAGGCTGCCGTGACTCGGACAGGATTAGACGGTCTC
GTGGGCGACGTGGAGTTGTATACGGATACCAACGGCCCTGGTGCCGTCTTCTTTGAAGG
AGTGCACGTCTCCCCACTAGTGCCGCCATCCGCAGCGGATGATCCGTCAGTATTTTGGG
TGCAGCATTGGACACCCCTTAGCCTGGATGTCAACCGTTCCAAATCTCGACTGTCGCCG
GAATGGATGGCCATGTTAGAAGGGTATGAGCGCCGGGCGTTCCTTGCACTGAAGGACA
TCCTCCAGCAGGTCACACCAGAGCTTCGTGCCACTTTTGACTGGCATCGTGAAAGCGTT
GTCAGTTGGATTGAGCACATTATGGAGGAAACCCGCGTGGGTCGGCACGCCGTCTGCAA
GCCTGAGTGGCTAGACCAAGAGCTAGAGAATCTCGGACACATATGGGGGCGGCCAGAC
GCGCGCATTGAGGATCGAATGATGTATCGAGTTTACCGGAACCTGCTACCCTTCCTCCG
CGGGGAAGCGAAGATGCTAGATGCTCTTCGGCAGGACGAATTGCTTACACAGTTCTATC
GCGACGAGCACGAGCTGCGCGATATCAACCGTCGACTGGGTCAGTTGGTTGGTGACCTA
GCCGTGCGCTTTCCACGTATGAAACTCCTTGAAGTCGGCGCCGGGACAGGCTCTGCCAC
TCGAGAGGTACTCAAACATGTCGGCCGGGCCTACCATTCCTACACGTTCACAGACATCT
CGGTTGGCTTTTTTGAAGACATGTTGGAAACAATTCCCGAGCACGCGGACCGTCTGCTA
TTCCAGAAGCTCGATGTCGGGCAAGACCCATTGCAGCAGGGCTTTGGTGAACACACTTA
CGATGTAATCATCGCCGCTAACGTACTTCATGCCACACCGACGCTGCAAGAGACTCTGC
GAAACGTGCGTCGTCTACTCAAGCCAGGAGGGTATCTGATCGCTCTGGAGATCACTAAC
ATTGATACAATCCGCATCGGCTTCTTGATGTGTGCCTTTGACGGCTGGTGGCTTGGCCGG
GAGGATGGCCGTCCATGGGGTCCGGTGGTCTCTGCATCACAGTGGGATAGCCTACTCCG
GGAGACGGGATTCGGTGGCATAGACACTATCACTGATCGCGCCGCTGACCAGCTCACC
ATGTACTCTGTCTTTGCCGCCCAAGCGGTGGACGACCAGATCACTCGATGTCGAGAACC
TCTGACGCCGCTCCCTCCTCAACCTCCTTTCTGCCGGGGAGTGATCATCGGAGGCTCGCC
TAGTCTGGTGACAGGCATAAGAGTCATTATTCATCCTTTCTTCTCGACTGTTGAACATGT
TTCTACCATCGAGAACCTGACGGAGGGAGCACCAGCTGTTGTGTTGATGTTGGCTGACC
TGAGCGACATCCCCTGCTTCGAAAATCTCACCGAGTCAAGACTGGCCGGACTCAAAGCA
CTGGTGCAAATGGCCGAGAAGACGCTCTGGGTGACCACGGGCTCTGAAGCGGACAACC
CTTATCTCTGCCTCAGCAAGGGCTTTCTCACTTCGATGAATTATGAACATCCAGCTATCT
TCCAATATCTGAACATCATCGACTCGGCTGACGTCCAACCCGTGGTCTTGGCCGAGCAT
CTTCTGCGATTGGCCTATACCAACCAAAACAATGACTTCGCCCTCACGAATTGCGTCCA
CAGCACAGAGCTTGAGCTGCGTCTCTACCAGGGCGGGATTCTGAAGTTCCCACGCATTA
ACGCGAGCGATGTCCTGAACAGTCGGTACGCGGCAGCTCGGCGCCCAGTCACCCATTCT
GTCACCAACATGCAGGACAGCGTGGTTGTACTTGACCAAAGCCCAAGTGGGAAGCTTC
GACTCGTGTTTGGGGAGGAGCTTGCAGGTGATCGCGCAACCGTCACCATTAACGTCCGA
TACTCGACCTCTCGTGCAATCCGCATCAATGGTGCTGGATATCTGGTCCTTGTTCTCGGG
CAGGATAAAGTTACCAAAGCGCGTCTGGTGGCTCTGGCAGGTCAGTCTGCGAGCGTCGT
CTCGTCCTCCTGTTATTGGGAGGTCCCAGCAGATATCTTCGAGGAGCAGGAGCCCGCGT
ATCTGTACGCCACAGCAACAGCTTTGCTCGCTGCCAGTTTGGTGCAGTCCAACGGCACC
ACAATCCTGGTACATGGCGCTGACATGGTCCTACGCCATGCAATCGCCATAGAGGCCGC
TTCACGGGTCATTCAGCCTATATTCACTACCACATCTCCCTCCGCAGCATCATCCGCGGG
TCTTGGGAAGAGCATCCTCGTGCATGAGAACGACACCCGGCGACAACTGGTTCATCTTC
TCCCTCGATATTTCACAGCTGCTGTGAATTTCGACCCTAGTGCCCGCCGACTCTTCGACC
GAATGATGACAGTCGGTCATCAATCGGGTGTCACAGAAGAACACCTTCTTACCACTTTG
ACAGCTGCCCTCCCTCGTCCGTCAGCATCTCTGCTGCCGGCCCAGCCTCAGGCTGCCAT
GGACACTCTTCGCAAAGCCTCATTGACTGCTTATCAGTTCACCGTCCAGTTGACAGCAC
CAGGACCCATCATCGCACCAATCGCCGACATCCAATCCTGTTCACAACAGTTAGCAGTC
GTAGACTGGAAACCATCTTGCGGCTCGGTTCCAGTACACCTCCAACCAGCCACTGAGCT
GGTTCGTCTCTCTGCTCAAAAGACATATCTCCTGGTGGGTATGACTGGTGCCCTCGGCCA
ATCCATCACGCAATGGCTGGTCACCCGCGGCGCTCGCAATATCGTCCTCACCAGCCGCA
AGCCATCAGTGGACCCCGCATGGATCGCAGAGATGCAGACCACAACAAGCGCGCGTGT
CCTCGTTACGCCAATGGATGTGACAAGCCGCGACTCGATCCTTGTGGTGGCACACGCCC
TGAAGGCCGACTGGCCGCCGCTCGGCGGCGTCGTCAACGGTGCCATGGTGCTCTGGGAC
CGTCTCTTCGTCGACGCACCCCTGTCCGTTCTGACGGGACAGCTCGCCCCAAAAGTCCA
GGGGAGCCTTCTCCTCGATGAGATTTTTGGCCATGAACCGGGCCTTGATTTCTTTATCCT
CTTCGGTAGCGCTATCGCCACTATTGGAAATCTGGGTCAGTCTGCCTACACAGCCGCCA
GTAACTTCATGGTCGCGCTTGCGGCGCAACGCCGCGCCCGAGGGCTTGTCGCAAGCGTC
CTCCAGCCGGCGCAGGTCGCCGGTGCCATGGGTTATCTCAGGGATAAAGACGACAGCTT
CTGGGCTCGGATGTTTGATATGATTGGGCGACATCTCGTCTCCGAACCAGATCTGCACG
AACTTTTGGCCCATGCTATCTTGTCGGGTCGTGGCCCTCCAGCTGACGTTGGATACGGAC
CAGGCGAGGATGAGTGCATCATTGGCGGACTCCGCGTCCAAGACCCTGCTGTATACCCA
GATATCCTCTGGTTCCGTACGCCCAAAGTCTGGCCATTCATCCACTATCACCACGAGGG
AACTGGCCCTTCATCTGGGGCGGCTGGTTCGATATCGCTGGTCGATCAGCTGAAGTGTG
CGACTAGCTTAGCCCAAGTTGGGGACATGGTGGAAGCTGGCGTTGCGGCCAAACTGCA
CCATCGACTCCATCTCCCAGGCGAGGTTGGAGGCGTCACTGGCGACACGCGTTTGACCG
AGCTGGGGGTGGACTCGTTAATTGCGGTGGACTTGCGTCGGTGGTTTGCGCAGGAGTTG
GAGGTTGATATTCCCGTTCTGCAGATGCTGAGTGGGTGTTCAGTAAAGGAGCTGGCTGC
TTCCGCGACGGCGTTGTTGCATCCGAAATTCTATCCGGAGGTGGTGGCCGATTCTGACG
TGGGGAGTGAGAGGGATGGTTCCTCGGACTCCCGTGGTGATACCTCTTCCTCCTCGTAT
CAGCTGATCACTCCGGAGGAGGGGGACCATGACTGA
ctvB (Aspergillus terreus) SEQ ID NO: 5
ATGACCTCCTACCAGCTTTCCGATGCCGAAGGCGCCGACNACTACTACAA
TCCACTTCTCTTGTGGTGGTACGACTTCTGGGTTCATTGGGTCAGCGCGCTCTTTGCCTG
GAAGTGTTCGTCTAAGGACATTCTTCTCCCTTTTTTCCTGTCCAACATTGGATCTCGGCA
CTGTGATGTTGGCGTGGGCACGGGCTACTATCTCTCTGCTGTGCGGAAGCGTCGGCCGT
CCTGGCCGGAGAAGCTGACGCTAGTGGATTTTCACATCCGATGCCTGAGGAAGGCTGCC
AACCGCGTTGGAGTAGCGGATCGCACGGAGTGTGTGCTGGCCAATATCCTGGAGCCCAT
CCCTATACAACCTGAACGGCAGTTCGACTCCATATCCCTTATGTACGTCCTGCACTGTCT
CCCGGGGACTTCTAAGGACAAAGGACGCGTGTTTGCCAACTTGAAGCCGCTCCTCAAGG
ACAATGGCACTCTCTTCGGGTCTACGCTGCTGTGCCGTGGAGTTCGGCAGAATTGGTTC
AGTTGGCTCCTTCAGCGCATCTACAATGCTGTGGACATGTTTCAGAATCGGTCAGACTA
TCCAGACGACTTTGTCCGTGCGCTGGAGGACGAGTTCGAGGAGGTGGAGAGCGTGATT
ATTGGAACGGTCCTTATGTTCAAGGCGCGGAAGCCTCGTCGCTAG
ctvC (Aspergillus terreus) SEQ ID NO: 6
ATGGAAGGAAAGCACCCTCAGTTCAAGGTCGTCATCGTCGGGGCATCCGT
CACGGGTCTGACCCTCGCCCACTGTCTGCATCGGGCCGGAATAGACTACGTGGTGCTGG
AGAAGCATCACGAAGTCCATCCGCCGATTGGCGCCGCGGTGGCCATCCTGCCGAATGG
AGGGCGGATCATGGAGCAGCTGGGGATCTTCCGGCACATTGAAGACCGCTGCCAGCCA
TTTCAAAGGGTGCATCTTTGCTTTCAGGATGGGTTCTACTATGATAGCCTTTCGCCTAGT
GTCGTCTTGAAGAGGTCGGTGCTTCCTTGGTCCCCGAGAGGGTGGAACAAAGTCGTAAG
AAGACGCTAACACGGCGGGTACGCAGGTTTGGCTTGAAATTCGCGTGCCTGGAACGGA
CTCAGCTACTCGAGATCCTGTACGCCCATTTGCCTGACAAGTCCAGGGTCCTCACGAGC
AAAGGCGTCGTCCGGATCACTCCCCATGGCAGCAAGATGACGGTCACCACCGCAGATG
GGGACGAGTTCCAGGGAGATCTGGTTGTCGGTGCGGACGGCGTGCACAGTGTGACGCG
GCGAGAGATGTGGCGCATTGCTAATATAGAGCAGCCGGGGCTGATTCCGCTCAAAGAA
CAGGCCAGTATGTTGCTACACTCCTTATCGATTCCGTCCAATATTAATAGATGATGCTCT
GCGGCTAGGCATGTCCGTCGAGTTCAGCTGCGTCTTCGGCATGTCCAACCCCATCCCCG
GACGGAAGCGTTGGCAGCATGTGATCCGCATCGGGCCCGGTTTCACGATCCTGATATTC
CCTGCGACCGGGGAGAGCCTTTTCTGGGTGCTGATTGAAAAGCTACCACACAAGTATAT
CTACCCTGACGTTCCGCGATTCTCGCAGGAGGACGCCATTGCGCGCTGTGAGGCAGCCG
CCAGCCAGCCTATCTGGGAGGAAGTACAATTCCGGGATATCTGGGCTCAACGCCGGGG
ATTCCGGATGGTTGCGTTGGAGGAGAATCTCTTCCGTACGTGGCATCATGGCCGGATCA
TCTGCATCGGGGATAGTATCAGCAAGGTATGTCTTGCATGGAGCCTTTTTTTCCCCTTCA
TATACTGATAGCTGGGACTCTAGATGACCCCCAACATCGGTCAAGGCGCCAATACAGCC
ATTGAAGCGGCGGCTGGACTGGCCAACGTGATATATGCGATTGCACAAAACCATGACC
AGCCGTCAAGTGACACCATTCACCAGGCGCTCGCTGCCTTCAGCGAGAAAAACCGCAA
ACGACTGGATGCTATCCATCTAGAGTCGCGCTGGATCACGCGATTGGAGGCTTGCCAGG
GACGGATGGTCACGGCCTTCACGCGATATGTGGCGCCACACTGTGGCGATCTTTTTGCG
TTGGGGGTGGTGCGTAACTCATACAACGGTGAGGTACTGCAGTTCTTGCCGTTGACTGA
ACGTTCGGGGAAGTACTGGCCGAAGTTGGAGTGGTGGAATACTTGGGGTCTGTCCAAGT
GGCAGGAATTTGGGGAGAGGGTGATGTATTGCTCTGGACTTGTGATAGTTTTGTGGATT
AGCTGGGTGGTGTTCAATGTCAAGGAAGTATGA
ctvD (Aspergillus terreus) SEQ ID NO: 7
ATGGCCCTTTCAGCCTACATACTTCTCTGCTTGTCCGTACTTGGACTAGAC
GCCATCTATGGCTTCNGATTCCGGAATGGCTTCCTCGAGCTCATGGCCAACAGCTACCG
AGAGCGAAAGCTATCCGGCACGGCAGAGCCCTTGCAAGGCAACATCACCGGAACGGGA
TTTGACGAACTCTTGGGAAATCTAATTGTCTTCTACTGGCCCGTGCTGGACGGACGCCAT
CCTGGTCTCAGTGTCCAGGCCTTTCACTTCTCTGGTGCAATCGTGGCCGTGTGGGTGGCG
ATCCAGGTCCAGAGCTGGAGATCGCCAACCCGCACCAGCGTTCTGCTATCGTGAGTGTC
TCTACCCTCGTATCCCTATTCTTCATCTGTCGGCCATGTTCCGAACCAACCGTCATAGTC
CAACCCTTTTCGCCATGCTATCCCAGGTCGTGGCCATCGCCGTCGTCGTACCCCTCTGGT
GCGCGATCCACATCTGGTCGTCGTCGTCGTCGTCGTCATCATCATCATCACGCCCAGTG
GCGCGCGTTATCTCGGCATCAGCAGCTCATTCCATACGCCTCATCCCCATCAGCATGAT
CCTGGGATTTGGAGTCCCGACTATCGCCATGGTCCTTCCGGAATCCGCGCACCAGGACT
TGTTCGGCAAACAGATGGCCATTGCCGCATGGCAGCTCTGGCCAATCTATGCAGCTCTG
TGTCACTGGGGTCTACGGGCCTTTTTCCGACCAAGAGCGAGCACGGGTATCTCCACGAG
GAGTGCATGCCGGACCGCGTATACCTTTGCGTTTGTATGCGCGATTATCCCCCATGTAAT
CAGCTGGGGTCTTTCCCTGACCTTGGCTCCTACAAATCTCTTGGCGGATGTATCCCCTTG
GCAGTTTGCTGGGGGTCGTACGGTCCAGGTTCAATCCATGGCCCAAGGGGCGCTGTGGT
TCCTACAGTGGGATCACCTCATTGGAATGGGGAGCTTTCTCCTGTGGGCACTGCATATG
CGGCGGACAGTTGAGAGGCAGTCGTCATTCACACAGACTTGCTATCTAGCGCTGAAAGT
TGGAGGTCTTTGTCTGATCTCAGGGCCCTGCGGTGCGGCTGTTTGGTTGCTGCGGGAGG
AATCTCAATTCTGA
pl-ggs (Clitopilus passeckerianus) SEQ ID NO: 8
ATGAGAATACCTAACGTCTTTCTCTCTTACCTGCGACAAGTCGCCGTCGAC
GCCACTCTGTCATCTTGCTCTGGAGTGAAGTCACGAAAGCCGGTCATTGCCTATGGCTTT
GACGACTCGCAAGACTCTCGCGTCGATGAGAATGACGAAAAAATATTGGAGCCCTTTG
GCTACTATCGTCATCTTCTGAAAGGCAAGAGCGCCAGGACGGTGTTGATGCACTGCTTC
AACGCGTTCCTTGGACTGCCCGAAGATTGGGTCATTGGCGTAACAAAGGCCATTGAAGA
CCTTCATAATGCATCCCTACTAATTGATGACATCGAAGACGAGTCTGCCCTCCGTCGTG
GTTCACCAGCTGCCCACATGAAGTACGGGATTGCGCTCACCATGAACGCGGGGAATCTT
GTCTACTTCACGGTCCTTCAAGACGTCTATGACCTTGGCATGAAGACAGGTGGCACACA
GGTTGCCAACGCAATGGCTCGCATCTACACTGAAGAGATGATTGAGCTCCATCGCGGTC
AGGGCATCGAAATCTGGTGGCGTGACCAGCGGTCCCCTCCCTCCGTCGATCAATACATT
CACATGCTCGAGCAGAAAACCGGCGGCCTGCTCAGGCTTGGCGTACGGCTCTTGCAATG
CCATCCCGGTGTCAATAGCAGGGCCGACCTCTCCGACATTGCGCTCCGTATTGGTGTCT
ACTACCAACTTCGCGACGACTACATCAACCTCATGTCCACAAGCTACCACGACGAGCGT
GGATTTGCTGAGGACATTACCGAAGGAAAGTATACCTTCCCGATGTTGCACTCTCTCAA
GAGGTCACCCGACTCTGGACTGCGTGAAATCTTGGACCTTAAGCCGGCCGACATCGCCC
TGAAAAAGAAAGCTATCGCTATCATGCAAGAGACAGGATCGCTTGTTGCAACCCGGAA
CCTTCTCGGTGCAGTCAGGAATGATCTCAGTGGATTGGTTGCTGAACAGCGTGGAGACG
ACTACGCTATGAGCGCGGGTCTTGAACGATTCTTGGAAAAGTTGTACATCGCAGAGTAG
pl-cyc (Clitopilus passeckerianus) SEQ ID NO: 9
ATGGGTCTATCTGAAGATCTTCATGCACGCGCCCGAACCCTCATGCAGAC
TCTCGAGTCTGCGCTCAATACGCCAGGTTCTAGGGGTATTGGCACCGCGAATCCGACTA
TCTACGACACTGCTTGGGTAGCCATGGTCTCCCGTGAGATCGACGGCAAGCAAGTCTTC
GTCTTCCCGGAGACCTTCACCTACATCTACGAGCACCAGGAGGCCGACGGCAGTTGGTC
AGGGGATGGGTCACTCATCGACTCCATCGTCAACACTCTGGCCTGCCTTGTCGCTCTCA
AGATGCACGAGAGCAACGCCTCAAAACCCGACATACCTGCCCGTGCCAGAGCCGCTCA
AAATTATCTCGACGATGCCCTAAAGCGCTGGGACATCATGGAGACTGAGCGTGTCGCGT
ACGAGATGATCGTACCCTGCCTCCTCAAACAACTCGATGCCTTTGGCGTATCCTTCAGCT
TCCCCCATCATGACCTTCTGTACAACATGTACGCCGGAAAACTGGCGAAGCTTAACTGG
GAGGCTATCTACGCCAAGAACAGCTCCTTGCTTCACTGCATGGAGGCATTCGTTGGTGT
CTGCGACTTCGATCGCATGCCTCATCTCCTACGTGATGGTAACTTCATGGCTACGCCATC
TACCACCGCTGCATACCTCATGAAGGCCACCAAGTGGGATGACCGAGCGGAGGATTAC
CTTCGCCACGTTATCGAGGTCTACGCACCCCATGGCCGAGATGTTGTTCCTAACCTCTGG
CCGATGACCTTCTTCGAGATCGTATGGTCGCTCAGCTCCCTTTATGACAACAACCTGGA
GTTTGCACAAATGGATCCGGAATGCTTGGATCGCATTGCCCTCAAACTACGTGAATTCC
TTGTGGCAGGAAAAGGTGTCTTAGGCTTCGTTCCCGGCACCACTCACGACGCTGACATG
AGCTCGAAGACCCTGATGCTCTTGCAAGTTCTCAACCACCCATATGCCCATGACGAATT
CGTCACAGAGTTTGAGGCACCTACCTACTTCCGTTGCTACTCTTTCGAAAGGAACGCAA
GCGTGACCGTCAACTCCAACTGCCTTATGTCGCTCCTCCACGCCCCTGATGTCAACATGT
ACGAATCCCAAATCGTCAAGATCGCCACCTACGTCGCCGATGTCTGGTGGACATCAGCA
GGTGTCGTCAAAGACAAATGGAATGTATCAGAATGGTACTCCTCTATGCTGTCTTCACA
GGCGCTTGTCCGTCTCCTTTTCGAGCACGGAAAGGGCAACCTTAAATCCATATCTGAGG
AGCTTCTGTCCAGGGTGTCCATCGCCTGCTTCACAATGATCAGTCGTATTCTCCAGAGCC
AGAAGCCCGATGGCTCTTGGGGATGCGCTGAAGAAACCTCATACGCTCTCATTACACTC
GCCAACGTCGCTTCTCTTCCCACTTGCGACCTCATCCGCGACCACCTGTACAAAGTCATT
GAATCCGCGAAGGCATACCTCACCTCCATCTTCTACGCCCGCCCTGCTGCCAAACCGGA
GGACCGTGTCTGGATTGACAAGGTTACATATAGCGTCGAGTCATTCCGCGATGCCTACC
TCGTTTCTGCTCTCAACGTACCCATCCCCCGCTTCGATCCATCTTCCATCAGCACTCTTCC
TACTATCTCGCAAACCTTGCCAAAGGAACTCTCTAAGTTCTTCGGGCGTCTTGACATGTT
CAAGCCTGCTCCCGAATGGCGCAAGCTTACGTGGGGCATTGAGGCCACTCTCATGGGCC
CCGAGCTCAACCGTGTCCCATCGTCCACGTTCGCCAAGGTAGAGAAGGGAGCGGCGGG
CAAATGGTTCGAGTTCTTGCCATACATGACCATCGCTCCGAGCAGCTTGGAAGGCACTC
CTATCAGTTCACAAGGGATGCTGGACGTGCTCGTTCTCATCCGCGGTCTTTACAACACC
GACGACTACCTCGATATGACCCTCATCAAGGCCACCAATGACGACTTGAACGACCTCAA
GAAGAAGATCCGCGACCTGTTCGCGGATCCGAAGTCGTTCTCGACCCTCAGCGAGGTCC
CGGATGACCGGATGCCTACGCACATCGAGGTCATTGAGCGCTTTGCCTATTCCCTGTTG
AACCATCCCCGTGCACAGCTCGCCAGCGATAACGATAAGGCTCTCCTCCGCTCCGAAAT
CGAGCACTATTTCCTGGCAGGTATTGGTCAGTGCGAAGAAAACATTCTCCTTCGTGAAC
GTGGACTCGACAAGGAGCGCATCGGAACCTCTCACTATCGCTGGACACATGTCGTTGGC
GCTGACAACGTCGCCGGGACCATCGCCCTCGTCTTCGCCCTTTGTCTTCTTGGTCATCAG
ATCAATGAAGAACGAGGCTCTCGCGATTTGGTGGACGTTTTCCCCTCCCCAGTCCTGAA
GTACTTGTTCAACGACTGCGTCATGCACTTCGGTACATTCTCAAGGCTCGCCAACGATCT
TCACAGTATCTCCCGCGACTTCAACGAAGTCAATCTCAACTCCATCATGTTCTCCGAATT
CACAGGACCAAAGTCTGGTACAGATACAGAGAAGGCTCGTGAAGCTGCTCTGCTTGAA
TTGACCAAATTCGAACGCAAGGCCACCGACGATGGGTTCGAGTACTTGGTCAAGCAACT
CACTCCACATGTCGGTGCCAAACGTGCACGGGATTATATCAATATCATCCGGGTCACCT
ACCTGCACACGGCACTCTACGATGACCTTGGTCGTCTCACTCGCGCTGATATCAGCAAC
GCCAACCAGGAGGTTTCCAAAGGTACCAATGGGGTTAAGAAAGCTAATGGGTCGGCGA
CAAATGGGATCAAGGTCACAGCAAACGGGAGCAATGGAATCCACCATTGA
pl-p450-1 (Clitopilus passeckerianus) SEQ ID NO: 10
ATGCTGTCCGTCGACCTCCCGTCTGTTGCGAACTTGGATCCCGTGATCGTG
GCTGCTGCTGCAGGATCCGCTGTTGCCGTCTATAAGCTCCTTCAGCTAGGCTCCAGGGA
GAACTTCTTGCCTCCCGGGCCACCTACCAAGCCTGTTCTCGGAAATGCTCATCTCATGAC
GAAGATGTGGCTTCCAATGCAATTGACACAGTGGGCCAGGGAGTATGGCGAAGTGTAC
TCTCTCAAATTGATGAATCGCACTGTGATTGTTTTGAACAGTCCAAAGGCTGTTCGGACT
ATTCTTGACAAGCAGGGTAATATCACAGGAGACCGGCCATTTTCGCCCATGATTGCCCG
GTATACTGAAGGCTTGAATCTCACGGTGGAAAGCATGGACACTTCTGTATGGAAGACTG
GTCGCAAAGGTATCCACAATTACCTAACGCCAAGTGCCTTGAGTGGCTACATAGCGCGA
CAAGAAGAGGAATCTGTGAACCTCATGCGCGATCTATTGATGGACGCTCCTAATCGGCC
GATCCATATTAGGCGCGCTATGATGTCGCTGCTCCTACACATTGTGTATGGCCAGCCAC
GCTGCGAGAGTTACTATGGCACAATTATCGAGAATGCATACGAAGCTGCCACCAGAATT
GGTCAAATCGCTCACAATGGTGCAGCGGTCGACGCTTTCCCCTTCTTAGACTACATTCCT
CGCGGTTTCCCCGGGGCCGGCTGGAAGACTATTGTGGACGAATTCAAGGATTTTCGTAA
TGGTGTCTACAATTCTCTCTTGGACGGTGCCAAGAAGGCGATGGATTCCGGTGTCAGGA
CCGGATGTTTTGCAGAGTCCGTGATTGACCATCCGGATGGTCGTAGCTGGATTGAGTTA
TCAAACCTTAGCGGTGGCTTCTTGGATGCCGGCGCGAAGACCACGATATCGTACATCGA
ATCGTGTATTCTTGCTCTTATCGCCCACCCGGACTGCCAGCGCAAGATACAGGACGAGC
TGGATAATGTTTTGGGGACCGAAACCATGCCGTGCTTCAATGATTTAGAGCGGTTGCCT
TATCTCAAGGCGTTCCTACAGGAGGTCCTTCGGATTCGGCCAGTCGGCCCTGTAGCCCT
TCCCCACGTCTCGCGGGAGAGCTTGTCTTATGGCGGTTACGTACTACCAGAGGGAAGTA
TGATCTTCATGAACATCTGGGGAATGGGCCACGACCCCGAGCTCTTCGACGAACCTGAG
GCCTTCAAGCCTGAACGCTATTTCTTGTCGCCAAACGGCACGAAGCCAGGCTTATCTGA
AGACGTCAATCCCGATTTCCTTTTCGGTGCTGGACGTAGAGTCTGCCCAGGCGATAAGC
TCGCGAAAAGATCAACTGGTCTCTTCATCATGAGGCTCTGTTGGGCATTCAATTTTTACC
CAGATTCTTCAAACAAGGACACTGTGAAGAATATGAACATGGAGGACTGTTACGACAA
GTCGGTTTCTCTTGAGACTCTTCCACTTCCGTTCGCATGCAAAATTGAACCTCGAGATAA
GATGAAGGAAGACTTGATTAAGGAAGCGTTCGCTGCGTTGTAG
pl-p450-2 (Clitopilus passeckerianus) SEQ ID NO: 11
ATGAATCTTTCTGCTCTGAAGGCTGCTCTGCTTGACAGCAACATGATCGCA
CCTGTGGCCATCCCTTTGGCATGCTACTTGGTCTACAAGCTGCTTCGTATGGGGTCGAGG
GAGAAGACGTTACCTCCTGGGCCACCTACGAAGCCGGTGTTGGGTAATCTCCACCAGAT
GCCAGCAATGGACGACATGCACCTTCAGCTTAGCCGATGGGCACAAGAATATGGAGGA
ATATACAGCTTGAAGATCTTCTTCAAGAACGTTATCGTCCTAACAGACTCAGCCTCCGTT
ACTGGCATTCTTGACAAGCTGAATGCCAAGACTGCTGAAAGACCCACTGGTTTCCTCCC
TGCTCCTATCAAAGACGACCGTTTCCTTCCTATCGCCTCCTACAAATCCGACGAATTCCG
AATCAACCACAAGGCCTTTAAGTTGCTCATTAGCAACGACAGTATTGATCGATATGCAG
AGAACATTGAGACGGAGACCATCGTGCTGATGAAGGAGCTGTTGGCTGAGCCCAAGGA
ATTCTTTAGGCATCTCGTCCGCACCAGCATGTCCAGTATTGTTGCTATCGCTTATGGTGA
ACGCGTCCTCACCTCCTCAGACCCATTCATTCCCTACCACGAAGAATATCTTCACGACTT
CGAAAACATGATGGGTCTCCGAGGTGTTCACTTCACCGCTCTAATTCCTTGGCTCGCCA
AGTGGCTTCCTGATAGTCTGGCCGGCTGGAGGGTCATGGCTCAAGGTATCAAGGACAAG
CAACTTGGTATCTTTAATGATTTCCTCGGAAGGGTTGAGAAGAGAATGGAAGCTGGCGT
CTTCGACGGGTCTCACATGCAGACCATTCTTCAGAGGAAGGATGAGTTTGGATTCAAGG
ATAGGGATCTTATTGCCTATCACGGAGGCGTCATGATTGACGGAGGAACTGATACCCTC
GCTATGTTCACTCGTGTCTTCGTGCTCATGATGACGATGCACCCCGAATGCCAGCAGAA
GATTCGTGATGAGCTGAAGGAGGTCATGGGCGATGAATACGACTCGCGTTTGCCAACTT
ATCAAGATGCATTGAAGATGAAATACTTCAATTGCGTCGTCAGAGAGGTAACTCGCATC
TGGCCTCCGAGTCCCATCGTACCGCCTCATTACTCGACAGAGGATTTCGAATACAATGG
CTACTTCATCCCGAAGGGTACCGTCATCGTGATGAACCTTTATGGCATCCAACGAGACC
CAAATGTTTTCGAGGCCCCAGACGATTTCCGCCCCGAACGGTACATGGAGTCTGAATTT
GGCACAAAACCAAGCGTTGACCTGACTGGCTACCGTCATACCTTCACTTTCGGCGCTGG
GCGCAGGCTCTGTCCTGGACTCAAGATGGCTGAAATTTTCAAGCGCACTGTATCTTTGA
ACATCATCTGGGGATTCGACATCAAGCCCCTGCCTAACAGCCCCAAGTCAATGAAGGAC
GATGTCGTTGTACCCGGTCCGGTCTCGATGCCAAAACCGTTTGAATGCGAGATGGTACC
ACGTAGTCAGTCAGTTGTGCAGGTGATCCACGATGTTGCAGACTATTAG
pl-sdr (Clitopilus passeckerianus) SEQ ID NO: 12
ATGGAAGGCAAGGTCGCAATCGTCACTGGCGCATCCAATGGTATTGGACT
CGCCACCGTCAATCTCCTCCTCGCAGCAGGAGCGTCTGTCTTTGGTGTAGACCTCGCTCC
AGCACCGCCCTCGGTGACCTCCGAGAAATTCAAATTCCTACAACTCAACATCTGCGACA
AGGATGCACCCGCTAGGATCGTATCCGGCTCCAAAGAGGCCTTTGGCATCGAGAGGATT
GATGCCCTCTTGAATGTCGCTGGTATTTCGGACTACTTCCAGACTGCGTTGACCTTCGAG
GACGATGTATGGGACCGAGTCCTCGATGTCAACCTGGCTGCACAAGTGAGGTTGATGAG
AGAGGTATTAAAGGTCATGAAGGTGCAGAAATCGGGGAGTATCGTGAATGTCGTCAGC
AAGCTGGCCCTCAGCGGTGCTTGTGGTGGTGTTGCATACGTTGCGAGTAAACATGCCTT
GCTTGGCGTGACGAAGAACACAGCCTGGATGTTCAAGGATGACGGCATTCGATGCAAT
GCAGTCGCACCTGGTTCGACTGACACCAACATCCGAAACACGACAGACCCGTCCAAAA
TAGATTACGACGCCTTCTCTCGAGCCATGCCTGTTATCGGCGTACACTGCAACTTGCAA
ACAGGTGAGGGCATGATGAGCCCTGAGCCTGCAGCCCAAGCGATCTTCTTCCTAGCTTC
AGACTTGAGTAAGGGCACGAACGGTGTCGTTATTCCAGTCGATAACGGGTGGAGTGTCA
TTTAG
Pl-p450-3 (Clitopilus passeckerianus) SEQ ID NO: 13
ATGGCTCCGTCAACGGAACGTGCTCTACCAGTCCTTGTAATATGGACTGCT
ATAGGCTTGGCCTACTGGATAGATTCTCAGAAGAAGAAAAAGCAGCACCTGCCGCCTG
GGCCAAAGAAACTTCCAATTATTGGCAACGTCATGGACCTACCAGCGAAGGTCGAATG
GGAAACCTATGCTCGCTGGGGTAAAGAGTACAACTCTGATATCATACATGTTAGCGCCA
TGGGAACCTCGATCGTAATACTGAATTCTGCCAACGCCGCCAATGACTTGTTGCTGAAG
AGGTCGGCGATCTACTCGAGCAGACCACACAGCACGATGCACCACGAGCTGTCAGGAT
GGGGCTTTACGTGGGCCTTAATGCCATACGGCGAGTCATGGCGGGCTGGTCGAAGAAG
CTTCACCAAGCACTTCAACTCTTCAAACCCCGGTATAAACCAACCTCGTGAGTTGCGAT
ATGTGAAACGGTTCCTCAAGCAGCTTTACGAGAAGCCCGACGACGTTCTCGATCATGTA
CGGAACTTGGTCGGCTCTACGACGCTTTCAATGACCTATGGCCTTGAGACTGAACCTTA
TAACGACCCCTATGTTGACCTGGTCGAGAAAGCTGTCCTTGCAGCGTCTGAGATTATGA
CGTCTGGCGCCTTTCTTGTTGACATCATCCCTGCGATGAAACACATTCCTCCATGGGTCC
CAGGGACTATCTTCCATCAAAAGGCTGCCTTAATGCGAGGTCATGCGTACTATGTTCGT
GAACAGCCATTCAAAGTTGCCCAGGAGATGATTAAAACTGGCGATTATGAGCCCTCCTT
TGNATCTGACGCTCTCCGAGATCTTCAGAACTCGGAAAACCAGGAGGCAGATTTGGAGC
ACCTCAAGGATGTTGCTGGTCAAGTCTACATTGCTGGTGCTGATACGACTGCATCCGCC
TTGGGGACTTTCTTCCTCGCCATGGTCTGTTTCCCCGAAGTACAGAAGAAAGCACAACG
AGAATTAGATAGTGTTCTCAATGGAAGGATGCCCGAGCACGCCGACTTCCCCTCTTTCC
CATACCTCAACGCTGTGATCAAGGAGGTTTACCGCTGGAGACCTGTGACTCCTATGGGC
GTACCTCATCAAACCATCTCAGATGACGTTTACAGGGAATACCACATCCCTAAGGGATC
CATCGTGTTTGCCAACCAATGGGCGATGTCCAACGACGAGACCGATTACCCCCAGCCAG
ACGAATTCCGGCCTGAGCGATACTTGACCGAAGACGGTAAGCCTAACAAGGCTGTCAG
AGACCCCTTTGATATCGCATTCGGCTTCGGTAGAAGAATTTGCGCTGGTCGTTACCTCGC
TCATTCCACCATCACCTTGGCTGCGGCCTCTGTTCTGTCGCTGTTTGATCTCTTAAAAGC
AGTTGACGAAAATGGCAAAGAAATTGAGCCTACTAGAGAGTATCACCAGGCTATGATC
TCACGTCCACTAGATTTCCCTTGCCGCATCAAGCCAAGAAGTAAGGAAGCTGAGGAGGT
CATCCGTGCTTGCCCGTTGACGTTCACGAAGCCTGCTAGTGGCTAG
Pl-atf (Clitopilus passeckerianus) SEQ ID NO: 14
ATGAAGCCCTTCTCACCAGAACTTCTGGTTCTATCTTTCATTCTATTGGTA
CTATCTTGTGCCATCCGGCCTGCTAGAGGACGATGGGTTCTCTGGGTCATTATTGTTGGG
CTCAACACCTACCTCACCCTGACTCCGACCGGCGATTCGACCTTGGATTATGACATTGC
CAATAACCTCTTCGTTATTACCCTCACGGCCACAGATTATATTCTCTTGACGGACGTCCA
GAGAGAGTTACAATTCCGCAACCAGAAAGGTGTCGAGCAAGCCTCGTTGCTTGAACGC
ATCAAGTGGGCGACCTGGCTGGTGCAAAGTCGGCGTGGTGTGGGCTGGAATTGGGAGC
CGAAGATTTTCGTCCACAAGTTTGACCCAAAGACTTCACGCCTTTCATTCCTCCTCCAGC
AACTCGTCACAGGTTTTCGGCATTACCTTATTTGCGATCTAGTCTCGCTATATAGCCGCA
GTCCAGTCGCCTTCATCGAACCTCTTGCTTCTCGCCCTCTGATCTGGCGGTGTGCAGATA
TTACCGCATGGCTCCTGTTCACGACGAACCAAGTATCAATTCTTCTTACGGCATTGAGTG
TCATGCAAGTTCTCTCAGGTTACTCAGAACCACAGGACTGGGTCCCCGTGTTTGGCCGC
TGGAGAGATGCTTATACCGTTAGGCGGTTCTGGGGTCGATCGTGGCATCAATTGGTTCG
CAGATGCCTATCAGCCCCAGGAAAACATCTTTCCACGAAGATTCTAGGCTTGAAGTCTG
GCTCTAACCCGGCGCTTTACGTACAACTGTACACCGCATTCTTCCTCTCGGGAGTTTTGC
ATGCGATTGGGGACTTCAAGGTTCACGCAGATTGGTACAAAGCCGGGACTATGGAGTTC
TTCTGTGTTCAAGCGGCGATCATACAGATGGAGGATGGGGTTCTCTGGGTCGGAAGGAA
GCTTGGTATCAAGCCGACTTCGTACTGGAAGGCCCTTGGACATCTTTGGACTGTGGCAT
GGTTCGTCTACAGCTGCCCGAATTGGCTGGGGGCAACTGTCTCGGGAAGGGGAAAGGC
CTCAATGTCGTTGGAGAGTAGTCTCATTCTTGGTCTGTACCGGGGGGAATGGAATCCCC
CTCGTGTAGCACAGTAG
AN7620 (egt1) (Aspergillus nidulans) SEQ ID NO: 15
ATGGGAACAGATTTACATCTCCTGCAGCCGGCAGCTTTAGACTTACCGTT
GCGGCCCTCCCAGTATGCTGCTGGTCCTATTCCCAGTCTGAAAGATTTTCAGGCACAGT
GGACCGCATGGGATATAGTCACCAAAGCAATGATCCCACAAGAAGAGCTGCTTTCAAA
GCCGATTAAACTACGAAATTCCCTGATATTTTACCTAGGACATATTCCCACGTTCGCTGG
TAAGTTCTATCGCAGACGGACTTCAATTGGGCTATTGCTTACTCAAGCTTTCAGATATTC
ACTTGACCCGTGCTTTGGGTGGCAAGCCTACAGACCCTAAATCATATCAACTGATATTT
GAGCGCGGCATCGACCCCGACGTTGACGATCCGGAAAAGTGCCATTCTCACAGTGAAA
TTCCGGACGAGTGGCCATCTCGTTCAGAAATCCTAGATTACCAAGACCGGGTCAGAAAC
AGGATACGATCCACACTCGAGATACCCGGCCTGTCCAAGAACAGAACGCTGGGTGAAG
CCCTTTGGATCGGGTTTGAGCACGAGGCAATGCACTTGGAGACCTTCCTCTACATGCTG
ATCCAGAGTGAGAGAGTCCTTCCTCCGCCGGGTATCCAGGCGCCGGATTTCAAGAAGAT
ATTTCTTGACGCAAGAGAAAATGCGAAGCCGAACGAATGGTTCGCTATACCAGAACAA
ACCCTATCTATAGGGCTTGACTCTTCAGAGGACTTGATGCCAACGTCAACATTCGGTTG
GGATAACGAAATACCTAAGCGCAATGTAACGGTACAACCATTTGAAGCGCAAGGGAGG
CCCGTTACCAATGGTGAATATGCCCGGTACCTTCAGAAGAACCAAATTCACAGGTGGCC
TGCCTCCTGGATTATGACCAATCAAGCAGATGGAGTTGCGAACGGTAGCACCGCAACTG
AGGATTTTCTAACTGACTTCTCTGTTCGCACGGTCTTCGGCCCCGTTCCACTTGAGCTAG
CTCAGGACTGGCCAGTCATTGCATCCTACGACGAGCTTCAACAATACGCTTCATGGATG
AATTGCCGCCTGCCGACGTTCGAGGAGGCCAAAAGCATCTATGCACACTCAGCTCGTCT
AAAGAAAACTCTTACAGACGGCTCCGCGGATGCAGCTAGCAACGGACTCAAAAACGGC
TACCACAAACCGCATCTTAGAAACGGCACCGTCAAAATTGCTCCTTCTACAGCCCAACC
CGTCCTCCCGCCCTCTGCATCCGCATCCTCTCCACCTGTTTTTCTCGACCTCACGACTTGC
AATGTCGGTTTCAAGAACTGGCATCCCATGCCCGTGATCCAGAATGGCGATAAACTGGC
TGGCCACGCAGAGCTCGGCGGCGTTTGGGAGTGGACTTCGTCACCTTTGGAGCCCCATG
ATGGGTTCAAAGCCATGGAGATTTACCCAGGCTATACTTGTTAGTTTTCCTTCTAGGCAC
ACTCGTCTGCATCATTCACTGACTTATTCTCTTCCTAGCCGACTTCTTCGACGGAAAGCA
TAATATAATCCTCGGTGGTTCGTGGGCGACTCATCCGCGGATTGTTGGGAGGACGACGT
TGTAAGTCCCATTATCGTATTCAGTTCCTGGAAAAGGCTAATACAGATGTAGTGTGAAC
TGGTATCAACGAAATTATCCTTACGCATGGGCTGGAGCGCGACTTGTACGGGACGTTTG
A
AN6227 (egt2) (Aspergillus nidulans) SEQ ID NO: 16
ATGGCCTCTCCAACTCCGTTTGGCGCCCCCATGAAAGCGCACTTCCTCCTC
GATCCTAATTACAACAATCTCAACCACGGTCTTTCCTCTCCTTCAACGTCGCTTACAGCA
ATTGCTGACACAACCAGGCTCTTTCGGCACCTACCCTTCCCAGGTCCTTGAAAAGCAAC
AGTCCATCCAGAAATCTCTCGAATCTAGGCCCGACATCTTCATCCGTTACATTCAGCCC
GGCCTTATCGACACTTCTCGGGCCGCCCTCGCGCCCCTCCTTAACGTCCCCGTCTCAGAC
CTCGTTTTGGTCAAAAATGCCACGACAGGTGTCAACACTGTCCTGCACAATCTCGCACT
GACCCGTACTCTGACTGCAGATGACGTTATCTTCTACTTTGACACCGTCTACGGCGCCGT
CGAGCGCGCCCTCTTTGCCCTAAAGGAATCCTGGGGCGTCAAGCTGAGGAAGGTTAAGT
ATGTTTTCCCCCTTGAAGAGGGGGGAATGGTCAAGAGGTTTAGGGAGGCACTGAAGAG
CGTGAGAAAAGAGGGCTTGACGCCGAAGCTCGCGGTCTTTGAGACGGTTGTGTCAAAC
CCAGGCATCAGGTTTCCCTTTGAAGAAATTACGAGGGCTTGTAAGGAAGAGGGTGTGCT
GAGTCTTATTGATGGAGCACATGCGGTTGGTATGATCAAGCTGGATCTGGCCGCGTTAG
GGGTGGACTTCTTCACGAGTAATTGTCATAAGTATGCCCTGTCTCTCCCTTTTCATTACC
CTAGCGCAGGCTTGATGGTTTATGCTTAGGTGGCTCTACACTCCGCGCTCCTGCGCAGTC
TTATACGTTCCCGAGCGCAACCAGAAGTTCATCCGGACAAGTCTCCCTACGTCATGGGG
GTATGTGCCGCCACAGGTTCCACCTTCTGAATCAGGCGAAGACAAGGATATCCCTCCGT
CCACGCTCCCAAATACTGGAAAATCCCCGTTCGTCGCGCTCTTTGAGTTCACAGGGACA
ACGGACGACAGCGCGTATGCGTGTGTCCCTGCAGCACTCAACTTCAGAGATGAGGTCTG
CGGTGGCGAGGAGAGGATCTATGCTTACCTTGAACGACTCGCTGGGGAGGCGGGTGAA
CTCCTTGCGTCTGCATTAGGGACGGATGTGTTGAGGGCTGTGAAAGATGCAAGCGGTGC
AGGGAAAGGTGGAAAGGGGCTGGGTTGCTCAATGGTCAATGTGCGGTTGCCTATTCGG
ATCACTAATCTGGACTTAAATTCTGGAACGAAGGATAAGGCGATAAATGTACGGCCGG
AGGACGTCAGTCCCCTGTCGCACTGGCTACACGAGCAGTTGATAGCCAGAGGGACATTC
GTACCGTGTTTTCCGCATGGGCAGTGGATGTTTGTCAGACTCAGCGCGCAAGTTTACCT
GGAAAGGAGTGATTTTGTTTGGTTGGGTGATGTCTTCAAGCATATAATGGAGGGTGTAC
CGGGCTTTTTGGAGGCGAACAAGAATGGAACTCAGGCCAAAGCGAAGATTTAG
Afu2g15650 (Aspergillus fumigatus) SEQ ID NO: 17
ATGTCCCCGTTGCCGTGTCCTTCGAAAAAGGTCGAGATTGTCGATATACAT
CGAAATGACGTGGAATTCTCGCTCGTGAATGAGATTCGCAAGGGCCTCAACCCTCCCGA
GGGTACTCCAAGATCATTGCCCACCATGCTGCTCTACGACGCTCAAGGGCTTAAGCTCT
TCGAGGAGATCACCTACGTGGATGAGTACTACCTGACCAACGCGGAAATCGAAGTGTT
GCAGAACCACTCCAAGAAGATTGTGGAACGCGTCCCCGAGAACGCACAATTATTGGAG
CTGGGTAGTGGGTATGATCTCTCCTGAAGAATGCTCTGACGGTTCTGTATCTAATCATCC
GTGCAGGAACCTTCGTAAGATCAAGATCCTTCTTCAAGAGTTCGAGCGGACAGGAAAG
CACGTCGACTACTATGCGTTGGATCTATCCCTGTCAGAGCTACAGCGGACCTTTGTCGA
GGTATCATCTGACGAGTACTCTCACGTTGATCTCCACGGTCTTCACGGGACCTATGATG
ATGCCCTTGCCTGGCTGAGCAATCCCCAGAACCTACAGCGTCCCACTGTGGTTATGTCT
ATGGGATCCTCAATTGGGAATTTCAGTCGTGAAGGTGCAGCAGAGTTTCTTGCCCAATT
TGCTCGGCTGCTCAAGCCATCCGATCTGATGATCATTGGGCTAGATGCTTGTACAGACC
CGGACAAGGTCTACAAGGCATACAACGATTCAAAGGGCATTACTCAGAGGTTCTACGA
GAATGGGCTATTACATGCGAATGCAGTCCTTGGATACGAGGCATTCCAACTTTCGGAAT
GGGAGGTGGTGACAGACTACGACGTTGCCGGGGGCCGACATCGAGCATTCTACTCACC
GAAGCAGAATGTTACAATCGATGGCGTGCTTCTGCAAAAAGGCGAGAAATTGGTTTTTG
AAGAGGCAACAAAGTATAGCCCTCAACAACGGGAGCAACTCTGGCGGGATGCAAACCT
CGTCCTTTGTGAGGAGCTTGGAATTAGCTCTGAGGAATACCGTAAGTTGGTCCTTGCCTC
CCGCAGCGCTGTTGTAGCAGAGATGTATGGGAGAAGCGTCGACTAATGAACGACCCTTC
ACTGACAGATATCCATCTGCTGTCACCGCCAACCCTCAGCCTTCCCAGTCAACCGTCCG
AATATGCTGCCAATCCAGTTCCGAGCTTCAAAGAATTCCAGTCATTGTGGACCGCTTGG
GACACTGTGACAAAGGCCATGGTGCCCCGCGAAGAACTCCTCGCGAAACCTATCAAGC
TTCGCAATGCATTGATCTTCTACTTTGGACACATCCCCACATTCACGGGTCAGTAATCCA
GAGACTTTTATTCAACGCCGTGAGCATGATACTGATGCTCTATTAGACATCCACTTGAC
ACGGGCTCTTGGTGGCTCTCCAACAGAACCACGAAACTATCGACAGATTTTCGAGCGTG
GGATTGACCCCGACGTAGACAATCCCGAGCACTGTCACTCCCATAGTGAGATTCCGGAT
GAATGGCCTCCCCTTGCTGAGATACTGGATTATCAGGATAGAGTCAGAAGTAGGGTTGA
TTCGGTTTTGCAGAGGGATGACATAACACAGAACCGCTGCCTGGGGGAAGCACTCTGG
ATTGGCTTCGAACACGAAGCGATGCACCTAGAGACCTTTCTCTATATGCTTTTGCAGAG
TGACAAGACCCTCCCACCGCCCCTTGCAGACAGACCAGATTTTGAGAAGTTGTTCCATC
AGGCCAGAGCGAACGCGAAACCAAATGAATGGTTTGCTATACCAGAACAGACATTATC
CATCGGTTTTGACGATACTGACGAACAATCTTTGCCTGATGTGTCTTTTGGATGGGACAA
TGAGAAGCCTCAGCGAACGATCACAGTTCGTGCTTTCGAAGCACAGGCTCATGCTATAA
CAAACGGCGAATATGCCAAATATCTGCAAGCGACTCGTCAGCGCCGGCGGCCAGAGTC
GTGGGTTCTAACTCATTCGGATGAGAATTATCCAATCTCCAAGGGGGTGACTCTGGAGA
GCAGCCAAGCCACAAAGGATTTCATGGACAACTTCGCTGTCCGCACCGTCTTTGGACCT
GTTCCGCTTGAATTCGCGCAGGATTGGCCTGTTATGGCTTCCTATGACGAACTGGCCCTA
TACGCCGAATGGGTGGGCTGCAGACTTCCCACTTATGAGGAGGTCAAGAGTATCTACAA
CTACTCCGCTCAGCTGAAGGAGACAAGACAACATGAGCCATCAGATCACGAGAGGTTC
GTAAATTCCCCTCGGTCTTTAAGTTTAGCTATGTCTGACAGAAGGCAGCAACGGTGTGA
AGGGCATTAACCGTGATATGGTGACGAACGGGCACTCGAAGGTTCACCAGGATAAGCC
CCGGACCCCAGAACGTCAGCCAATCCAACCTCCTTCCCAAAGCACAATGCCAGTATTTG
TTGACCTCCACGGTTGCAACGTTGGGTTCAAGCACTGGCACCCTACCCCGGTCATCCAG
AACGGCGATCGACTCGCCGGTCACGGTGAGCTGGGAGGAGTCTGGGAATGGACCAGCA
CGCCACTGACGCCACATGATGGCTTCAAGGCCATGGATATTTATCCAGGTTATACAGGT
AAGCTGTTGACCTACTTGGTGGATTTCTATAAACCCAACTAACCAGCAATAGCGGACTT
CTTTGATGGAAAGCACAACATTGTCCTGGGTGGTTCATGGGCCACGCATCCTAGAATTG
CGGGACGGACAACATTGTAAGTTATCACCTGTGATGTCGACCTGAATATGCACTAACTG
TCTCAAGCGTCAACTGGTATCAGCACAACTATCCTTACACTTGGGCGGGAGCACGTCTC
GTGCGCAGCCAGTAG
Afu2g13295 (Aspergillus fumigatus) SEQ ID NO: 18
ATGGGGAGGCTCCAGCTCTCAAGCGGGCTGAAGGCCATAGCCCTGCTCAC
ATTCGCAGCGACAGCAACATGCTGGCCATACGACGAGTCCCTCGTTGACTACAACGTCA
ACACGAATAAGTCGGCCACTAACCCCGCCGACTACTGGGGAGAATGGTCGGATCACAA
GTACCATCCGTCGCCAGAGAACTGGCGGTTTCCGTTTTACACACTCTTCATGGACAGATT
CGTGAATGGGGATCCAACAAATGATAACATCAACGGGACCACGTTTGAGCACGATCTC
AATTCAAATCAGATGCGTCATGGCGGTGATGTTGCTGGGCTGGTTGATACGCTGGATTA
CTTGCAAGGGATGGGGATCAAGGTGCGTTGCCGCTATTATTCGTCAATTTCTGGTAAAG
ATATAGTTGATGCTAACGATGCGATAGGGACTGTACCTTGCTGGAACTCCGCTCATGAA
CCAGCCCTGGGGCTCGGATGGGTATTCGGCGCTCGACACCACCCTTCTCGACCAGCACT
TTGGCACGATCCAGGTCTGGCGCGACGCCATCACCGAAATCCACAAACGGGGGATGTA
CGTTCTGTTCGACAATACAATCGCTACGTGAGTACCTCGCCTCCCTGCGTTTTGCCATCG
GTTTGTAAAAAACTTGGGCCTGACACTAGATTACTGCAGCCTCGGTGATCTCATTGGTTT
TGAAGGTCATCTCAACGACACCACGCCCTTCTCCGAGAAAGAACACAAGGCCCTTTGGA
AGTCCAACAGACGGTACGTCGATTTCGATATTGGCAACACCTACAACGCCACCTGTGAT
TATCCGCGCTTCTGGTACGAGGACGGGATGCCAGTCAATGAGTCCCTGACCGCGGGCCT
GGTCGGGTGTTATGATAGTGACTTTGACCAGTACGGTGACATCGAAGCGTTCGGTGTCT
GGCCAGACTGGAAGCGTCAACTGGCGAAGTTTGCGTCCGTCCAGGATCGGTTGAGAGA
ATGGTACCCACCTGTACGGGAACGGCTGATCCGACACACATGCATGATCATTGCCTCCT
TTGATATCGACGGTATTCGGTACGACAAGGCGACCCAGGCGACTGTCGATGCGTTGGGG
GACATGTCCAAAGCGTATCGGGAATGCGCGCGGGCTGTCGGCAAGGAGAACTTTTTCAT
TGCGGGTGAGATCACAGGAGGCAATACTTTTGGGTCTATCTATCTCGGACGAGGACGGC
AGTCGAACCAGGTCGATTCGGTGGGGAATATCTACGACGCCATGAAACTGACAAACGA
GTCGGATCCGCAGCTTTTCCTGCGCGAGGTCGGCCACGAGGCTATCGACGCCGGTGCCT
TCCATTACTCGACTTACCGTGCCCTGACCCGCTTCCTGGGAATGGACGGGCAGCTGGAG
GCCGGTTATGACGTCCCTCTTGACTGGGTGCAGGCATGGGGAAACATGACCGTGACCAA
CGACCTGATTAACGCCAACACGGGCAAGTTCGATCCCCGGCACATGTACGGCGTGACA
AACCAGGATGTTTTCCGTTGGCCGGCAATCGAGTGGGGTGTTGAGAGGCAGATGCTGGG
CTCGTTTATCACCACGCTGATGCTGCCGGGCATCCCGCTGCTGCTTTGGGGAGAGGAGC
AAGCGTTCTACGTGCTCGATGCGACGGCGTCAAACTACATATATGGACGACAGGCCATG
TCGTCTGCGACCGCGTGGAAGACGCACGGCTGCTTCTCACTCGAATCGAGCCAGTACTA
CCAGTGGCCCCTGGTGGCCGCACTCGACGGTTGCAACGACGAGACGGTCACCTACGAC
CACCGGGACCCGAGCCACCCGGTGCGCAACATCATCAAGCACATGTACCAGATGCGCG
AGCAGTACCCAATGCTCAACGATGGGTTCATCATCGAGACCCTTTCTAACCAGACGGAA
CCTGTCTACTACCCCGGTTCCAACGGGACCGAGACCGAGACAGGCATGTGGTCGGTCCG
CCGTGACCGGAACGAAGAGACCCAGGACTTTGGCTCGAGTGACGACAACGAACCCATC
TGGCTAGTCTATAGCAACATGAACCGCACGCACGACTACACATTTGATTGCTCTGACAA
TGAGACGGCACTCATTGCCGCCTTCCCCTCAGGCACCAAGGTCAGGAACCTCTTTCACC
CGTACGACACGCTGACCCTAGGCGACGGGCCAAAGGAAATGGTCTATGGCAACTCGAC
TGAGCTGGTAGGCTGCTTACCGAACCTGACGCTTAGCCGGTACGAGTTCCGTGCCTATG
TCAAGAACGAGCTCTGGAAAAAGCCCCGGCCGATGATCACCAAGTTCCAGCCCGGCGA
CGATGAGGCCAACGGCCACGACAGCCCGCTGCGTTCGACCGTCGCACCAGATGCGTCC
GAGACGGTGCGACTGACGCTGCAGTTTTCTGAGGCGATGGGATGCGATTCTGTCACAGA
CTCCATCTCATTTAACTCTTCCACGGAAACCGGCAAGATTCCATCCATCGACGCCTCCAC
CGTCCAGTGTGGGAACATCACTGAAGTCGCCAACAGCAACGCTACCGGGCACATCCCC
GGTAAGTGGCAGTGGGCTGCAGATCTGAGAGGGGTGTACAACGGCATCCACCGGGTCA
CTGTCAACAACGCCAGCAACGCCGACGGGGACGACTCCACCCACGCAGTCGACCACTT
CCTCTTCCGGATTGGGCAGATTGACAACCCCATGGTCTTTACCTCTGCCAACTACTCCAG
CAGTTTGCTGCATGAGAAGGAGGACGGCACGTTCTATATCCAACACCACGCTGCCGGTG
CTGACAAGTACCGCTATTCGACGAATTGGGGCACCACGTTCTCTGACTGGAAAACGTAC
AAGGGAGGTAACGACACGATTACCATGCTTCCATGGAATGGCACTAAGGCGCAGGAAT
GGGAGGGACATCACATCCGCGTCGAGTACTGGTCCCGCTGGACTGGCAGCAGTAGCCA
CGTCCAGGAAGGCGATTCCGGCTGGAAGTACAAGACGCCACGTCGCTTCCCACATGCCT
TTTTCAATGGGCCCTACAACCAGTACGGGTACGACGGCGGTCTGGACAACCAGATCAA
GCTGGACGCGGGCGCCGGCGGCGACGGATACTGGAAGTACCATTTCACCTCGGAGTGG
CCTGCCGTCGGGCAGGTGAATGTCTGGGGCATCAATCCGGATGGCGAGCCCGATCAGA
GCTGGGTGATGGGCGACGTGGACGGGGACAAGGTCCTCGACCGCATGCCACCAAGCGC
GCTCTCTGCAACGCTGATTAACATCACTGATCACCCAACGCACCCTTATATCTCGTGGA
AGCTGTACATAAACGACGCGACGATGCGGTACTATCTCATCCCCGCTGGCCACCAGAGC
GGGCAGATCGCCATGTTCGTCCTCTTCTGGATCATCCCTCTCCTGTCCGGCTCCGCCTGC
GTCTATATCTTCATGAAGTCCTTCTACAAGGTCAAATTCAACGAAATCGGCGCTGCCGG
CGCAAGCACGGAAATGAAGTCACTTGTCCCGCTTGCTCTCCGTCGACGCATGAAGCAGC
TCGCCTCTGGGAATGGCAAGAACGGTCCGTCATTCAACCCGCTCATGCGCCTTGCTGAA
AAGTCCGGCTTTATGCAGAGCACGACTGCGTTGGCCGGTGCGGCCTCGGGCAAGAGAC
GCATGGTCCTGATCGCGACCATGGAATACGATATTGAAGACTGGGGGATCAAGATCAA
GATCGGTGGTCTGGGAGTGATGGCCCAGCTGATGGGTAAGACACTCGGCCACCAGGAC
CTTATCTGGGTGGTGCCTTGTGTCGGGGGCGTGGACTATCCTGTTGATACCCCTGCGGA
ACCCATGACGGTCACTATCCTGGGTCAGGCATACCAGGTGAACGTGCAGTACCACGTTC
TGAAGAACATCACGTACGTCTTGCTTGACGCGCCCGTCTTCCGCCAGCAGACCAAGTCG
GAGCCGTACCCAGCGCGCATGGATGACTTGGACTCTGCAGTGTATTACTCCGCATGGAA
TCAGTGTATCGCAGAGGCGATCAAACGGTTCCCTGTTGATTTGTACCATATTAACGATT
ACCACGGCTCTGTCGCGCCGCTGTACCTCCTGCCAGGTACCATCCCCGCCTGTCTGTCAC
TTCACAATGCCGAGTTTCAGGGTCTATGGCCGATGCGAACACAGAAGGAGAAAGAGGA
GGTCTGTTCCGTGTTCAACCTGGATGTTGAGGTTGTCCGCAACTACGTCCAGTTTGGTGA
GGTGTTCAACCTCCTCCACGCAGGAGCGAGCTATCTCCGCGTTCACCAACAGGGCTTTG
GTGCCGTCGGTGTCTCGAAGAAGTACGGCAAGCGTTCATATGCTCGGTACCCGATCTTC
TGGGGCCTGCGCAAGATCGGAAACCTGCCGAACCCCGATCCGTCTGATGTGGGCGAGT
GGACGAAGGAGGACAGCCTGATCAAGGACGAGGATATCAAGGTGGATCCAGAGTTTGA
AGCAGGCCGCGCTGAGCTCAAGAGACAGGCGCAGGAGTGGGCTGGGCTGGACCAGAA
CCCTGATGCTGACCTCCTGGTTTTCGTCGGTCGTTGGTCCATGCAGAAGGGTGTTGACCT
TATCGCAGACGTTATGCCCGCAGTCCTTGAAGCACGACCAAACGTACAGCTCATCTGTG
TTGGACCTGTTATCGATCTCTATGGCCGCTTTGCGGCGCTCAAGCTCGACCGCATGATGA
AGGTGTATCCCGGCCGCGTCTTCTCACGCCCTGAATTCACTGCCCTGCCGCCGTATATCT
TCTCCGGCGCGGAATTCGCGCTTATTCCCTCCCGTGACGAACCCTTTGGTCTTGTCGCCG
TCGAGTTTGGCCGCAAAGGTGCGCTCGGCATTGGAGCCCGTGTTGGTGGACTGGGACAG
ATGCCTGGATGGTGGTACAATGTCGAATCGGTCTCGACATCCCATCTCCTCATGCAGTTC
AAGCTCGCTATTGAGGCTGCCCTTTCGTCAAAGACTGAGACACGTGCTATGATGCGTGC
TCGCTCTGCTAAACAACGCTTCCCCGTTGCGCAGTGGGTGGAAGATCTGGAGATCCTAC
AGTCGACTGCTATCCAGGTTCATGAGAAGGAGGTTTCCCGTGGTCATGCAGGTGGCCGC
CCCATGACACCGATGACGCCCTCTGGAGCTACGACGCCCAGCGGAATGATGACGCCTA
CTACAGGATCCCGTGGTCTTAAGCCTCTGTCCCAGGGCGTCGGCATGGGCCTCTCGGTG
CCACACTCCCGGGAGAGCAGCTATTCGAACCTCAACCGATTGAGCGAGTACGTTGCGCA
AAAGACTCCGGGCGAGTCTCAACCGCGAGAATCGTCCGGTCTGCAGCGCTCGCTCTCGC
TCGGTGTCCGGTCCGGTCCTGGCCACCGTGGTCGTGCGCGCAAGCAGAAACCCGGCGCG
GACAACATCCCGGAGGGCAACGAGGACGGCAGCAGCAGTGATACTGAGTCTATCCCTG
ACTACTACGACGACGAGTACACCCTCACCCCAGCTCAGATTGAAGAAAGCAGACGGGC
TCAGGCCACCCGTTCCATATCCTTCAGCCCCGAGACACTCCAGCCACCTCGATCTCCCCT
GCCCGCACCTCCCATGAGTCCCGGGACGCCACCGTCGGTTGAGCAGACTCTGCTGCCTC
CACCAAAGCCCTTCGCTGCCGCTGACGCTGGAAACAGACTTAGTAGTGCATCAGTGTTA
TCCCTAGACTCTGTCGTGGGTGGGAAGAAAGACTTCAAGCTCCAAAAGGTCGACCCCTT
CTTTACTGACAGCAACGGCGAGTATGCTCGGAACTTCGAACAGCAGCTCGAGAACCTCA
ACGGCTCCAACTCCGAGTCACAGCTGTGTATCGAGGAGTTCCTTGTCAAATCGGAACGC
CGCTGGTTCAACAAGTTCCGCGATGCAAGGCTGGGTCGTCTGCGCTCGCCCACACCGTC
TGTCTTCCGCGACAACCACAGCCACGGCCGGGGCTCGCCCGATGGATCTATGTATGTGG
ATGAGGCAGGCCACCGTAATAGTGGCGATGCCGTGCACGACAACGGCTCAGATGACAC
AGACGATGAGTTCCTCCTAGGCAAAGACTACGTGCCTCCCACGGGCCTGAAGAAATGG
ATGCAGATCAAGATCGGCGACTGGCCGGTGTACACGCTCTTCCTCGCACTGGGCCAGAT
CATCGCTGCAAACTCCTACCAGATCACGCTGCTGACAGGCGAAGTCGGCCAAACAGCC
GAGAAACTGTACGGAATTGCCACAACCTATGCCATAACCTCAGCCCTCTGGTGGCTCGT
CTTCCGGTACTTCAAATCCATCGTCTGCCTCTCCACCCCCTGGTTCTTGTATGGAATTGC
GTTCCTCTTCATTGGCTCCGCGCACTTTGAGTCCGACTCATTCACCCGCGGCTGGATCCA
GAATGTCGGCAGCGGCTTCTATGCTGCGGCTTCCTCCAGCGGATCCATCTTCTTTGCGCT
GAACTTCGGTGATGAGGGCGGTGCACCAGTGAGCAAGTGGATTTTCCGTGCATGTGTGA
TTCAGGGCATCCAGCAGGTCTATGTCATTGTGCTCTGGTATTGGGGTTCCACGATGGCA
CACCAGTCCAGCCAGGGTCTGCTCACGGCGGATAATACGATCTCAAATACGTGGAAGA
TGACGTACGTCCAAAACGACCCTAAATTACCCCCAATTAATATACAGAACTCACTAACA
AACTGATACAGCGCCATTTGCTATCCGATCGCCATGTTACTGTGGGCCATCGGCCTCCT
GCTGATCTTCGGCCTACCAAACTACTACCGCCAGAAACCCGGCAAAGTACCCTCCTTCT
ACAAATCCCTTTTCAGACGCAAGATCGTGCTCTGGAACTTTGTCGCCGTCATCCTACAG
AACTTCTTCCTGTCGGCTCCCTATGGCCGCAACTGGAGTTTCCTCTGGACATCCTCCCAC
ACAAAGCCCTGGCAGATTGTCATCCTGTGTGTCATATTCTTTGGTCTTCTGTGGTGCGCG
TTCCTTTACATTGTCGCTGTTCTTTCGAAGCAGCACTCCTGGTTTTTGCCAGTCTTCGCAT
GCGGGCTCGGCGCCCCCCGCTTCCTGCAAATCTGGTGGGGTGTTTCTGGCATCGGACAC
TACCTTCCCTGGGTTGCCGGCGGGTATACTGGCGGCGCACTAGTTAGTCGGAGTATCTG
GCTGTGGCTAGGTGTGCTAGATTCAATCCAGGGGTTAGGGTTCGGAATCATTCTCCTCC
AAACCCTGACCCGGATGCATATGTGCTTTACGTTGATCGTCAGCCAAGTCCTGGGATCT
ATAGCAACCATCGTTGCGAGGGCATGTGCTCCAAATAATGTTGGTCCGGGACCTGTGAG
TCCAGATATCACGAAGGGAGCGGGCGAACTGGCGAACGCCTGGTTCTGGGTTGCATTGT
TCTGTCAGTTGCTTGTCTGTGCTGGGTTCTTACTGTTTTTCCGCAAGGAGCAACTTGCGA
AGCCTTAA
NCU04343 (Neurospora crassa) SEQ ID NO: 19
ATGCCGAGTGCCGAATCCATGACCCCAAGCAGTGCCCTCGGACAGCTCAA
AGCAACTGGACAACATGTGCTATCCAAGCTTCAGCAGCAGACATCAAACGCCGATATC
ATCGACATCCGCCGCGTTGCTGTAGAGATCAACCTCAAGACCGAGATAACCTCCATGTT
CCGACCTAAAGATGGCCCTAGACAGCTACCCACCTTGCTTCTCTACAACGAGAGAGGCC
TGCAGCTGTTCGAGCGTGTAAGTTTATCCAAGTTGTGGGACTGACATGCTGGTTCATACT
GACCCTTATCAGATCACATACCTTGAAGAGTACTATCTTACCAATGACGAGATCAAAAT
CCTCACCAAACATGCGACCGAAATGGCTAGCTTCATCCCGTCAGGTGCCATGATCATTG
AGCTCGGAAGCGGGTAAACATCTGACAATTCGACAATGGGGTCCTGGATTTCGAAGAG
CTGACCATATCCCCAGAAATCTGCGCAAAGTAAACCTTCTATTGGAAGCCCTAGACAAC
GCCGGCAAGGCAATTGACTATTATGCCCTTGACCTGTCTCGGGAGGAGCTGGAGCGCAC
TCTCGCTCAGGTACCATCCTACAAGCACGTCAAGTGCCACGGTCTTCTGGGTACATATG
ACGATGGACGTGACTGGCTCAAGGCCCCAGAGAACATCAATAAACAGAAATGCATCTT
GCACCTCGGGTCAAGCATTGGTAAGCTTGAATACGTATTGAATTGGTCAAAGAAAATCC
TGACATGATCGTGTCTCTAGGCAACTTTAACCGCAGTGACGCCGCTACCTTTCTCAAGG
GCTTTACGGACGTCCTTGGACCCAATGACAAGATGCTCATTGGGGTTGACGCTTGCAAT
GACCCGGCGAGGGTATAGTACGTGTTCGACAAACATCTTGGGACGTCATCAGGAACATT
GCTAACCATATACTAGCCACGCTTACAACGGTTTGTGCTTACTCATGTGACATGGTATA
AGACCACCTTCAAGCTGACAATAACCAAACAGACAAGGTTGGTATTACTCACGAGTATG
TCATACCCCTTTTTCTGTCGCTCATGGCGGCTCCCGCTTTCCAGCCCATTACTGATACTG
ATGATTCAGGTTCATCTTGAATGGTCTTCGCAACGCCAATGAAATTATCGGAGAGACGG
CCTTCATCGAGGGCGATTGGAGAGTCATTGGCGAATATGTGTATGACGAAGAGGGCGG
CAGACACCAGGCCTTTTACGCCCCCACTCGCGACACCATGGTTATGGGGGAGTTGATTA
GGTCACACGACAGGATCCAGATCGAACAGAGCCTAAAGTACTCGAAAGAGGAGTCAGA
GAGGCTCTGGAGCACGGCGGGATTGGAACAAGTCTCGGAATGGACGTACGGCAACGAA
TATGGTAAGTTGCTTTCACAGCTTTTCCTTGCTTGAGGTTTCCAGCTTTTTCTGAGACTAC
CCGTTTGAGCCGTCCGGCTGGTGTTACCACACCTTCCCCACACCCTACGGACCAGTCGT
ATGATGATATGATGTTGCGGTGTCTCCTGATGGGCACAGCTCCTACACTGGCACAAGTT
TACCAATGTGTAGGCTGGTGTTGGGCTGCAGTGTTCGGGTAGCGCGCACGGGCTGGGGA
GGCTCGAATATTTGCGCCGTCAAAACCATTGACACATGCTGACATCCCTCCGTCGTACT
GCAGGACTCCATCTGCTTGCCAAGTCAAGGATGTCTTTCAGTCTCATCCCTTCGGTGTAC
GCTCGCAGCGCACTCCCAACTCTGGACGACTGGGAGGCCCTTTGGGCGACATGGGATGT
CGTCACACGTCAGATGCTTCCCCAGGAAGAGCTTCTGGAGAAGCCCATCAAGCTCCGAA
ACGCCTGCATCTTTTACCTCGGTCACATCCCGACCTTCCTCGACATCCAGCTCACAAAGA
CCACCAAGCAGGCTCCGTCAGAGCCCGCTCACTTTTGCAAGATCTTCGAGCGAGGCATT
GATCCTGATGTCGACAACCCGGAGCTGTGTCATGCGCACTCGGAGATTCCTGATGAATG
GCCGCCGGTGGAAGAAATCCTGACCTACCAGGAGACGGTACGGTCCCGGTTACGCGGC
CTCTATGCGCATGGCATCGCGAATATTCCGCGGAATGTGGGTCGGGCCATTTGGGTTGG
GTTTGAGCACGAGCTTATGCATATCGAGACGCTGTTGTACATGATGCTACAGAGCGACA
AGACGCTGATCCCAACCCATATTCCACGGCCCGACTTTGACAAGCTCGCGAGGAAGGC
AGAGTCCGAGAGGGTTCCCAATCAGTGGTTTAAGATTCCGGCACAGGAGATCACCATCG
GTTTGGATGATCCTGAGGATGGATCTGATATCAACAAGCATTATGGCTGGTAAGTGGAC
CTTTTTTCCGTCAAAGTCGACGGTGGATTAACTTGCAAATAGGGACAACGAGAAGCCTC
CAAGGCGCGTTCAAGTTGCTGCCTTTCAGGCTCAAGGGAGGCCGATCACCAACGAAGA
GGTACGTTTGCCTAGCAACTATTATTGCACGATACTAACAACAACCAGTACGCGCAATA
TCTGCTTGAAAAGAACATCGACAAGCTCCCTGCCTCTTGGGCCCGCCTGGACAACGAGA
ACATTAGCAATGGAACAACAAACAGCGTGAGCGGTCACCACAGCAACAGAACCTCCAA
GCAGCAGCTCCCTTCATCTTTCCTCGAGAAGACAGCAGTCCGCACAGTCTACGGTCTCG
TGCCTCTCAAGCACGCTCTCGACTGGCCCGTGTTTGCCTCTTACGACGAACTTGCCGGTT
GCGCAGCTTACATGGGCGGCCGTATTCCCACCTTCGAAGAGACCCGGAGCATTTACGCT
TACGCCGATGCTCTCAAGAAGAAGAAGGAAGCTGAGAGACAATTGGGAAGGACGGTTC
CGGCTGTTAATGCGTATGTCCCTTCGACTTACTCTCCCGTGTCCAGCTCGCACTAACAAT
ATCTTTCCCATTGATAAACAGCCACCTAACCAACAACGGCGTGGAAATCACTCCCCCAT
CCTCTCCCTCTTCCGAGACCCCCGCCGAGTCTTCCTCCCCCTCCGACAGCAACACCACCC
TCATCACCACCGAAGACCTCTTCTCTGACCTAGACGGTGCCAATGTCGGTTTTCACAACT
GGCACCCTATGCCCATCACCTCCAAAGGCAACACCCTTGTCGGGCAAGGCGAGCTCGGC
GGCGTGTGGGAATGGACTTCATCGGTCCTCCGCAAGTGGGAGGGGTTCGAGCCGATGG
AGCTGTACCCCGGCTATACGGCGGATTTTTTCGATGAGAAGCACAACATTGTGCTGGGA
GGGAGCTGGGCTACGCATCCGAGGATTGCGGGGAGGAAGAGCTTTGTGAATTGGTACC
AGAGGAATTATCCTTATGCTTGGGTGGGGGCGAGAGTTGTTAGGGATTTGTGA
NCU11365 (Neurospora crassa) SEQ ID NO: 20
ATGGTCGCCACCACCGTCGAGCTGCCTCTGCAGCAAAAGGCCGACGCCGC
CCAAACTGTTACTGGCCCCCTCCCATTCGGCAATTCCCTCCTCAAGGAATTCGTCCTCGA
CCCTGCCTACCGGAACCTCAACCATGGTCTGTACAACATCCCCCATGCATGACCCCAAT
GTAATCACTAACGAAAAGCAGGCTCCTTCGGCACCATCCCCTCCGCCATCCAACAAAAA
CTCCGCAGTTACCAAACCGCCGCCGAAGCCCGCCCCTGCCCCTTCCTCCGCTACCAAAC
CCCCGTACTCCTCGACGAATCCCGCGCCGCCGTCGCCAACCTCCTCAAAGTCCCCGTCG
AAACCGTCGTCTTCGTCGCCAACGCCACTATGGGCGTCAACACTGTCCTGCGCAACATC
GTCTGGTCCGCCGACGGCAAGGACGAGATCCTCTACTTCGACACCATCTACGGCGCCTG
CGGCAAGACCATCGACTACGTCATCGAAGACAAGCGAGGGATCGTTTCTTCTCGCTGTA
TCCCATTGATCTACCCCGCCGAAGACGACGATGTCGTCGCTGCCTTCCGGGACGCCATC
AAGAAGAGCCGCGAAGAAGGCAAGCGACCCCGTCTGGCTGTTATCGACGTCGTCTCCT
CCATGCCTGGCGTACGGTTCCCGTTCGAGGACATCGTCAAGATCTGCAAAGAAGAAGA
GATCATCTCGTGCGTGGACGGCGCCCAAGGCATCGGCATGGTGGACCTCAAGATCACC
GAGACCGACCCGGATTTTTTGATTAGTAACTGCCACAAGTGGCTGTTTACTCCGCGCGG
ATGTGCCGTGTTCTACGTGCCTGTGCGTAACCAGCACTTGATCCGCTCGACGCTGCCTAC
TAGCCATGGGTTCGTGCCGCAGGTCGGGAATAGGTTCAACCCGCTGGTGCCGGCGGGG
AACAAGTCAGCGTTTGTTAGCAACTTTGAGTTTGTGGGCACGGTGGATAACTCGCCGTT
CTTTTGTGTTAAGGACGCGATCAAGTGGCGCGAGGAGGTGCTCGGTGGGGAGGAGAGG
ATCATGGAGTACATGACTAAATTGGCGAGGGAAGGTGGACAGAAAGTGGCGGAGATTC
TGGGGACGAGGGTGTTGGAGAATAGCACGGGAACGCTGATCAGGTGCGCCATGGTCAA
TATTGCGTTGCCGTTCGTTGTGGGAGAGGATCCCAAGGCGCCGGTCAAGTTGACGGAGA
AGGAGGAGAAGGATGTTGAAGGGTTGTATGAGATTCCCCATGAGGAGGCAAACATGGC
GTTCAAGTGGATGTACAACGTGCTGCAGGACGAGTTTAACACGTTTGTACCCATGACCT
TCCACAGGAGGAGGTTCTGGGCCAGATTGAGCGCGCAGGTGTATTTGGAGATGAGCGA
TTTCGAGTGGGCGGGGAAGACGTTGAAGGAGTTGTGTGAGAGGGTGGCTAAGGGGGAG
TACAAGGAGAGCGCCTGA
AN3307 (agsB) (Aspergillus nidulans) SEQ ID NO: 21
ATGGGGAGGCTCCAGCTCTCAAGCGGGCTGAAGGCCATAGCCCTGCTCAC
ATTCGCAGCGACAGCAACATGCTGGCCATACGACGAGTCCCTCGTTGACTACAACGTCA
ACACGAATAAGTCGGCCACTAACCCCGCCGACTACTGGGGAGAATGGTCGGATCACAA
GTACCATCCGTCGCCAGAGAACTGGCGGTTTCCGTTTTACACACTCTTCATGGACAGATT
CGTGAATGGGGATCCAACAAATGATAACATCAACGGGACCACGTTTGAGCACGATCTC
AATTCAAATCAGATGCGTCATGGCGGTGATGTTGCTGGGCTGGTTGATACGCTGGATTA
CTTGCAAGGGATGGGGATCAAGGTGCGTTGCCGCTATTATTCGTCAATTTCTGGTAAAG
ATATAGTTGATGCTAACGATGCGATAGGGACTGTACCTTGCTGGAACTCCGCTCATGAA
CCAGCCCTGGGGCTCGGATGGGTATTCGGCGCTCGACACCACCCTTCTCGACCAGCACT
TTGGCACGATCCAGGTCTGGCGCGACGCCATCACCGAAATCCACAAACGGGGGATGTA
CGTTCTGTTCGACAATACAATCGCTACGTGAGTACCTCGCCTCCCTGCGTTTTGCCATCG
GTTTGTAAAAAACTTGGGCCTGACACTAGATTACTGCAGCCTCGGTGATCTCATTGGTTT
TGAAGGTCATCTCAACGACACCACGCCCTTCTCCGAGAAAGAACACAAGGCCCTTTGGA
AGTCCAACAGACGGTACGTCGATTTCGATATTGGCAACACCTACAACGCCACCTGTGAT
TATCCGCGCTTCTGGTACGAGGACGGGATGCCAGTCAATGAGTCCCTGACCGCGGGCCT
GGTCGGGTGTTATGATAGTGACTTTGACCAGTACGGTGACATCGAAGCGTTCGGTGTCT
GGCCAGACTGGAAGCGTCAACTGGCGAAGTTTGCGTCCGTCCAGGATCGGTTGAGAGA
ATGGTACCCACCTGTACGGGAACGGCTGATCCGACACACATGCATGATCATTGCCTCCT
TTGATATCGACGGTATTCGGTACGACAAGGCGACCCAGGCGACTGTCGATGCGTTGGGG
GACATGTCCAAAGCGTATCGGGAATGCGCGCGGGCTGTCGGCAAGGAGAACTTTTTCAT
TGCGGGTGAGATCACAGGAGGCAATACTTTTGGGTCTATCTATCTCGGACGAGGACGGC
AGTCGAACCAGGTCGATTCGGTGGGGAATATCTACGACGCCATGAAACTGACAAACGA
GTCGGATCCGCAGCTTTTCCTGCGCGAGGTCGGCCACGAGGCTATCGACGCCGGTGCCT
TCCATTACTCGACTTACCGTGCCCTGACCCGCTTCCTGGGAATGGACGGGCAGCTGGAG
GCCGGTTATGACGTCCCTCTTGACTGGGTGCAGGCATGGGGAAACATGACCGTGACCAA
CGACCTGATTAACGCCAACACGGGCAAGTTCGATCCCCGGCACATGTACGGCGTGACA
AACCAGGATGTTTTCCGTTGGCCGGCAATCGAGTGGGGTGTTGAGAGGCAGATGCTGGG
CTCGTTTATCACCACGCTGATGCTGCCGGGCATCCCGCTGCTGCTTTGGGGAGAGGAGC
AAGCGTTCTACGTGCTCGATGCGACGGCGTCAAACTACATATATGGACGACAGGCCATG
TCGTCTGCGACCGCGTGGAAGACGCACGGCTGCTTCTCACTCGAATCGAGCCAGTACTA
CCAGTGGCCCCTGGTGGCCGCACTCGACGGTTGCAACGACGAGACGGTCACCTACGAC
CACCGGGACCCGAGCCACCCGGTGCGCAACATCATCAAGCACATGTACCAGATGCGCG
AGCAGTACCCAATGCTCAACGATGGGTTCATCATCGAGACCCTTTCTAACCAGACGGAA
CCTGTCTACTACCCCGGTTCCAACGGGACCGAGACCGAGACAGGCATGTGGTCGGTCCG
CCGTGACCGGAACGAAGAGACCCAGGACTTTGGCTCGAGTGACGACAACGAACCCATC
TGGCTAGTCTATAGCAACATGAACCGCACGCACGACTACACATTTGATTGCTCTGACAA
TGAGACGGCACTCATTGCCGCCTTCCCCTCAGGCACCAAGGTCAGGAACCTCTTTCACC
CGTACGACACGCTGACCCTAGGCGACGGGCCAAAGGAAATGGTCTATGGCAACTCGAC
TGAGCTGGTAGGCTGCTTACCGAACCTGACGCTTAGCCGGTACGAGTTCCGTGCCTATG
TCAAGAACGAGCTCTGGAAAAAGCCCCGGCCGATGATCACCAAGTTCCAGCCCGGCGA
CGATGAGGCCAACGGCCACGACAGCCCGCTGCGTTCGACCGTCGCACCAGATGCGTCC
GAGACGGTGCGACTGACGCTGCAGTTTTCTGAGGCGATGGGATGCGATTCTGTCACAGA
CTCCATCTCATTTAACTCTTCCACGGAAACCGGCAAGATTCCATCCATCGACGCCTCCAC
CGTCCAGTGTGGGAACATCACTGAAGTCGCCAACAGCAACGCTACCGGGCACATCCCC
GGTAAGTGGCAGTGGGCTGCAGATCTGAGAGGGGTGTACAACGGCATCCACCGGGTCA
CTGTCAACAACGCCAGCAACGCCGACGGGGACGACTCCACCCACGCAGTCGACCACTT
CCTCTTCCGGATTGGGCAGATTGACAACCCCATGGTCTTTACCTCTGCCAACTACTCCAG
CAGTTTGCTGCATGAGAAGGAGGACGGCACGTTCTATATCCAACACCACGCTGCCGGTG
CTGACAAGTACCGCTATTCGACGAATTGGGGCACCACGTTCTCTGACTGGAAAACGTAC
AAGGGAGGTAACGACACGATTACCATGCTTCCATGGAATGGCACTAAGGCGCAGGAAT
GGGAGGGACATCACATCCGCGTCGAGTACTGGTCCCGCTGGACTGGCAGCAGTAGCCA
CGTCCAGGAAGGCGATTCCGGCTGGAAGTACAAGACGCCACGTCGCTTCCCACATGCCT
TTTTCAATGGGCCCTACAACCAGTACGGGTACGACGGCGGTCTGGACAACCAGATCAA
GCTGGACGCGGGCGCCGGCGGCGACGGATACTGGAAGTACCATTTCACCTCGGAGTGG
CCTGCCGTCGGGCAGGTGAATGTCTGGGGCATCAATCCGGATGGCGAGCCCGATCAGA
GCTGGGTGATGGGCGACGTGGACGGGGACAAGGTCCTCGACCGCATGCCACCAAGCGC
GCTCTCTGCAACGCTGATTAACATCACTGATCACCCAACGCACCCTTATATCTCGTGGA
AGCTGTACATAAACGACGCGACGATGCGGTACTATCTCATCCCCGCTGGCCACCAGAGC
GGGCAGATCGCCATGTTCGTCCTCTTCTGGATCATCCCTCTCCTGTCCGGCTCCGCCTGC
GTCTATATCTTCATGAAGTCCTTCTACAAGGTCAAATTCAACGAAATCGGCGCTGCCGG
CGCAAGCACGGAAATGAAGTCACTTGTCCCGCTTGCTCTCCGTCGACGCATGAAGCAGC
TCGCCTCTGGGAATGGCAAGAACGGTCCGTCATTCAACCCGCTCATGCGCCTTGCTGAA
AAGTCCGGCTTTATGCAGAGCACGACTGCGTTGGCCGGTGCGGCCTCGGGCAAGAGAC
GCATGGTCCTGATCGCGACCATGGAATACGATATTGAAGACTGGGGGATCAAGATCAA
GATCGGTGGTCTGGGAGTGATGGCCCAGCTGATGGGTAAGACACTCGGCCACCAGGAC
CTTATCTGGGTGGTGCCTTGTGTCGGGGGCGTGGACTATCCTGTTGATACCCCTGCGGA
ACCCATGACGGTCACTATCCTGGGTCAGGCATACCAGGTGAACGTGCAGTACCACGTTC
TGAAGAACATCACGTACGTCTTGCTTGACGCGCCCGTCTTCCGCCAGCAGACCAAGTCG
GAGCCGTACCCAGCGCGCATGGATGACTTGGACTCTGCAGTGTATTACTCCGCATGGAA
TCAGTGTATCGCAGAGGCGATCAAACGGTTCCCTGTTGATTTGTACCATATTAACGATT
ACCACGGCTCTGTCGCGCCGCTGTACCTCCTGCCAGGTACCATCCCCGCCTGTCTGTCAC
TTCACAATGCCGAGTTTCAGGGTCTATGGCCGATGCGAACACAGAAGGAGAAAGAGGA
GGTCTGTTCCGTGTTCAACCTGGATGTTGAGGTTGTCCGCAACTACGTCCAGTTTGGTGA
GGTGTTCAACCTCCTCCACGCAGGAGCGAGCTATCTCCGCGTTCACCAACAGGGCTTTG
GTGCCGTCGGTGTCTCGAAGAAGTACGGCAAGCGTTCATATGCTCGGTACCCGATCTTC
TGGGGCCTGCGCAAGATCGGAAACCTGCCGAACCCCGATCCGTCTGATGTGGGCGAGT
GGACGAAGGAGGACAGCCTGATCAAGGACGAGGATATCAAGGTGGATCCAGAGTTTGA
AGCAGGCCGCGCTGAGCTCAAGAGACAGGCGCAGGAGTGGGCTGGGCTGGACCAGAA
CCCTGATGCTGACCTCCTGGTTTTCGTCGGTCGTTGGTCCATGCAGAAGGGTGTTGACCT
TATCGCAGACGTTATGCCCGCAGTCCTTGAAGCACGACCAAACGTACAGCTCATCTGTG
TTGGACCTGTTATCGATCTCTATGGCCGCTTTGCGGCGCTCAAGCTCGACCGCATGATGA
AGGTGTATCCCGGCCGCGTCTTCTCACGCCCTGAATTCACTGCCCTGCCGCCGTATATCT
TCTCCGGCGCGGAATTCGCGCTTATTCCCTCCCGTGACGAACCCTTTGGTCTTGTCGCCG
TCGAGTTTGGCCGCAAAGGTGCGCTCGGCATTGGAGCCCGTGTTGGTGGACTGGGACAG
ATGCCTGGATGGTGGTACAATGTCGAATCGGTCTCGACATCCCATCTCCTCATGCAGTTC
AAGCTCGCTATTGAGGCTGCCCTTTCGTCAAAGACTGAGACACGTGCTATGATGCGTGC
TCGCTCTGCTAAACAACGCTTCCCCGTTGCGCAGTGGGTGGAAGATCTGGAGATCCTAC
AGTCGACTGCTATCCAGGTTCATGAGAAGGAGGTTTCCCGTGGTCATGCAGGTGGCCGC
CCCATGACACCGATGACGCCCTCTGGAGCTACGACGCCCAGCGGAATGATGACGCCTA
CTACAGGATCCCGTGGTCTTAAGCCTCTGTCCCAGGGCGTCGGCATGGGCCTCTCGGTG
CCACACTCCCGGGAGAGCAGCTATTCGAACCTCAACCGATTGAGCGAGTACGTTGCGCA
AAAGACTCCGGGCGAGTCTCAACCGCGAGAATCGTCCGGTCTGCAGCGCTCGCTCTCGC
TCGGTGTCCGGTCCGGTCCTGGCCACCGTGGTCGTGCGCGCAAGCAGAAACCCGGCGCG
GACAACATCCCGGAGGGCAACGAGGACGGCAGCAGCAGTGATACTGAGTCTATCCCTG
ACTACTACGACGACGAGTACACCCTCACCCCAGCTCAGATTGAAGAAAGCAGACGGGC
TCAGGCCACCCGTTCCATATCCTTCAGCCCCGAGACACTCCAGCCACCTCGATCTCCCCT
GCCCGCACCTCCCATGAGTCCCGGGACGCCACCGTCGGTTGAGCAGACTCTGCTGCCTC
CACCAAAGCCCTTCGCTGCCGCTGACGCTGGAAACAGACTTAGTAGTGCATCAGTGTTA
TCCCTAGACTCTGTCGTGGGTGGGAAGAAAGACTTCAAGCTCCAAAAGGTCGACCCCTT
CTTTACTGACAGCAACGGCGAGTATGCTCGGAACTTCGAACAGCAGCTCGAGAACCTCA
ACGGCTCCAACTCCGAGTCACAGCTGTGTATCGAGGAGTTCCTTGTCAAATCGGAACGC
CGCTGGTTCAACAAGTTCCGCGATGCAAGGCTGGGTCGTCTGCGCTCGCCCACACCGTC
TGTCTTCCGCGACAACCACAGCCACGGCCGGGGCTCGCCCGATGGATCTATGTATGTGG
ATGAGGCAGGCCACCGTAATAGTGGCGATGCCGTGCACGACAACGGCTCAGATGACAC
AGACGATGAGTTCCTCCTAGGCAAAGACTACGTGCCTCCCACGGGCCTGAAGAAATGG
ATGCAGATCAAGATCGGCGACTGGCCGGTGTACACGCTCTTCCTCGCACTGGGCCAGAT
CATCGCTGCAAACTCCTACCAGATCACGCTGCTGACAGGCGAAGTCGGCCAAACAGCC
GAGAAACTGTACGGAATTGCCACAACCTATGCCATAACCTCAGCCCTCTGGTGGCTCGT
CTTCCGGTACTTCAAATCCATCGTCTGCCTCTCCACCCCCTGGTTCTTGTATGGAATTGC
GTTCCTCTTCATTGGCTCCGCGCACTTTGAGTCCGACTCATTCACCCGCGGCTGGATCCA
GAATGTCGGCAGCGGCTTCTATGCTGCGGCTTCCTCCAGCGGATCCATCTTCTTTGCGCT
GAACTTCGGTGATGAGGGCGGTGCACCAGTGAGCAAGTGGATTTTCCGTGCATGTGTGA
TTCAGGGCATCCAGCAGGTCTATGTCATTGTGCTCTGGTATTGGGGTTCCACGATGGCA
CACCAGTCCAGCCAGGGTCTGCTCACGGCGGATAATACGATCTCAAATACGTGGAAGA
TGACGTACGTCCAAAACGACCCTAAATTACCCCCAATTAATATACAGAACTCACTAACA
AACTGATACAGCGCCATTTGCTATCCGATCGCCATGTTACTGTGGGCCATCGGCCTCCT
GCTGATCTTCGGCCTACCAAACTACTACCGCCAGAAACCCGGCAAAGTACCCTCCTTCT
ACAAATCCCTTTTCAGACGCAAGATCGTGCTCTGGAACTTTGTCGCCGTCATCCTACAG
AACTTCTTCCTGTCGGCTCCCTATGGCCGCAACTGGAGTTTCCTCTGGACATCCTCCCAC
ACAAAGCCCTGGCAGATTGTCATCCTGTGTGTCATATTCTTTGGTCTTCTGTGGTGCGCG
TTCCTTTACATTGTCGCTGTTCTTTCGAAGCAGCACTCCTGGTTTTTGCCAGTCTTCGCAT
GCGGGCTCGGCGCCCCCCGCTTCCTGCAAATCTGGTGGGGTGTTTCTGGCATCGGACAC
TACCTTCCCTGGGTTGCCGGCGGGTATACTGGCGGCGCACTAGTTAGTCGGAGTATCTG
GCTGTGGCTAGGTGTGCTAGATTCAATCCAGGGGTTAGGGTTCGGAATCATTCTCCTCC
AAACCCTGACCCGGATGCATATGTGCTTTACGTTGATCGTCAGCCAAGTCCTGGGATCT
ATAGCAACCATCGTTGCGAGGGCATGTGCTCCAAATAATGTTGGTCCGGGACCTGTGAG
TCCAGATATCACGAAGGGAGCGGGCGAACTGGCGAACGCCTGGTTCTGGGTTGCATTGT
TCTGTCAGTTGCTTGTCTGTGCTGGGTTCTTACTGTTTTTCCGCAAGGAGCAACTTGCGA
AGCCTTAA

What is claimed is:

1. A non-human organism for upgrading intermediate oxidation products formed by catalytic degradation of linear and/or branched alkanes, polystyrenes, or mixtures thereof, wherein the non-human organism is genetically modified to convert the intermediate oxidation products to secondary metabolites.

2. The non-human organism of claim 1, wherein the intermediate oxidation products include C₁₀₋₂₅ aldehydes, C₁₀₋₂₅ esters, C₁₀₋₂₅ compounds having ring systems, C₄₋₂₀ Carboxylic acids or dicarboxylic acids, or combinations thereof.

3. The non-human organism of claim 1, wherein the linear and/or branched alkanes are polyethylenes or polypropylenes, or mixtures thereof.

4. The non-human organism of claim 1, wherein the linear and/or branched alkanes are provided as new or used motor oil-based materials.

5. The non-human organism of claim 1, wherein the secondary metabolites include ergothionine.

6. The non-human organism of claim 1, wherein the secondary metabolites include asperbenzaldehyde.

7. The non-human organism of claim 1, wherein the secondary metabolites include citreoviridin and mutilin.

8. The non-human organism of claim 1, wherein the non-human organism is a fungus.

9. The non-human organism of claim 1, wherein the non-human organism is Aspergillus nidulans.

10. The non-human organism of claim 9, wherein the non-human organism is genetically modified by removing the afoD gene.

11. The non-human organism of claim 9, wherein the non-human organism is genetically modified by replacing the promoter of the afoA promoter with the alcA promoter (alcA(p)) in the nuclear genome.

12. The non-human organism of claim 9, wherein the non-human organism is genetically modified by replacing the promoter of the alcR gene with the constitutive gpdA promoter in the nuclear genome.

13. The non-human organism of claim 9, wherein the non-human organism is genetically modified by removing the afoD gene, replacing the promoter of the afoA gene with the gpdA promoter and inserting an additional copy of the afoA gene under control of the afoE promoter in the nuclear genome thereby forming a positive feedback loop that generates high levels of both AfoA and asperbenzaldehyde.

14. The non-human organism of claim 9, wherein the non-human organism is genetically modified by deleting the entire sterigmatocystin biosynthetic gene cluster (genes stcA-stcW) and the emericellamide biosynthetic gene cluster (genes easA-easD) in the nuclear genome.

15. The non-human organism of claim 9, wherein each heterologously expressed gene in the non-human organism is placed under control of alcA(p) in the nuclear genome.

16. The non-human organism of claim 9, wherein the non-human organism is genetically modified by amplifying genes AN7620 and AN6227 using A. nidulans genomic DNA and inserting AN7620 and AN6227 replacing the coding regions of afoG and afoF, respectively and replacing the native promoter of afoA with alcA(p) to create strain YM267 and wherein maintenance of the native promoters of each of these genes allows for the protein AfoA to bind to native promoters and drive expression of the genes AN7620 and AN6227.

17. The non-human organism of claim 16 further comprising replacing the coding regions of afoE and afoD with the A. fumigatus egt1 (Afu2g15650) and egt2 (Afu2g13295) homologs, respectively, to yield strain YM812.

18. The non-human organism of claim 17 further comprising inserting a third pair of ergothioneine biosynthetic genes into the regulon by replacing the coding regions of afoC and afoB with the N. crassa egt1 (NCU04343) and egt2 (NCU11365) genes to yield strain YM820.

19. The non-human organism of claim 18 further comprising deleting the agsB gene encoding an α-1,3-glucan synthase to create strain YM847.

20. A method comprising:
catalytically degrading linear and/or branched alkanes or polypropylene in an oxidizing environment to form intermediate oxidation products with a catalyst system that includes one or more catalysts; and
contacting the intermediate oxidation products with a non-human organism, wherein the non-human organism is genetically modified to convert the intermediate oxidation products to secondary metabolites.

21. The method of claim 20, wherein the one or more catalysts include a transition metal-containing catalyst.

22. The method of claim 20, wherein the one or more catalysts include MeReO₃ and oxides and halides of Co, Mn, Cu, and Re.

23. The method of claim 20, wherein the one or more catalysts include Fe(acac)₂ or Fe(acac)₃

24. The method of claim 20, wherein the catalyst system includes a cocatalyst.

25. The method of claim 20, wherein the cocatalyst includes hydroxylated amines.

26. The method of claim 20, wherein the cocatalyst includes hydroxysuccinamide (NHS) or hydroxylamine.

27. The method of claim 20, wherein the co-catalyst includes N-hydroxyphthalimide (NHPI).

28. The method of claim 20, wherein the co-catalyst includes include NO.

29. The method of claim 20, wherein the intermediate oxidation products include C₁₀₋₂₅ aldehydes, C₁₀₋₂₅ esters, C₁₀₋₂₅ compounds having imbedded ring systems, benzoic acid, C₄₋₂₀ Carboxylic acids or dicarboxylic acids, thereof.

30. The method of claim 20, wherein the linear and/or branched alkanes are polyethylenes or polypropylenes, or mixtures thereof.

31. The method of claim 20, wherein the linear and/or branched alkanes are provided as motor oil.

32. The method of claim 20, wherein the secondary metabolites include ergothionine.

33. The method of claim 20, wherein the secondary metabolites include asperbenzaldehyde.

34. The method of claim 20, wherein the secondary metabolites include citreoviridin and mutilin.

35. The method of claim 20, wherein the non-human organism is a fungus.

36. The method of claim 20, wherein the non-human organism is Aspergillus nidulans.

37. The method of claim 36, wherein the non-human organism is genetically modified by removing the afoD gene.

38. The method of claim 36, wherein the non-human organism is genetically modified by replacing the promoter of the afoA promoter with the alcA promoter (alcA(p)) in the nuclear genome.

39. The method of claim 36, wherein the non-human organism is genetically modified by replacing the promoter of the alcR gene with the constitutive gpdA promoter in the nuclear genome.

40. The method of claim 36, wherein the non-human organism is genetically modified by removing the afoD gene, replacing the promoter of the afoA gene with the gpdA promoter and inserting an additional copy of the afoA gene under control of the afoE promoter in the nuclear genome thereby forming a positive feedback loop that generates high levels of both AfoA and asperbenzaldehyde.

41. The method of claim 36, wherein the non-human organism is genetically modified by deleting the entire sterigmatocystin biosynthetic gene cluster (genes stcA-stcW) and the emericellamide biosynthetic gene cluster (genes easA-easD) in the nuclear genome.

42. The method of claim 36, wherein each heterologously expressed gene in the non-human organism is placed under control of alcA(p) in the nuclear genome.

43. The method of claim 36, wherein the non-human organism is genetically modified by amplifying genes AN7620 and AN6227 using A. nidulans genomic DNA and inserting AN7620 and AN6227 replacing the coding regions of afoG and afoF, respectively and replacing the native promoter of afoA with alcA(p) to create strain YM267 and wherein maintenance of the native promoters of each of these genes allows for the protein AfoA to bind to native promoters and drive expression of the genes AN7620 and AN6227.

44. The method of claim 43 further comprising replacing the coding regions of afoE and afoD with the A. fumigatus egt1 (Afu2g15650) and egt2 (Afu2g13295) homologs, respectively, to yield strain YM812.

45. The method of claim 44 further comprising inserting a third pair of ergothioneine biosynthetic genes into the regulon by replacing the coding regions of afoC and afoB with the N. crassa egt1 (NCU04343) and egt2 (NCU11365) to yield strain YM820.

46. The method of claim 45 further comprising deleting the agsB gene encoding an α-1,3-glucan synthase to create strain YM847.

47. A method for making a biocontrol agent comprising:
culturing a strain of Aspergillus flavus with benzoic acid in a culture medium; and
collecting spores from the strain of Aspergillus flavus therefrom.

48. The method of claim 47, wherein the strain of Aspergillus flavus is A. flavus Af36.

49. The method of claim 47, wherein the benzoic acid is formed by catalytically degrading a polystyrene in an oxidizing environment to form intermediate products with a catalyst system that includes one or more catalysts.

50. The method of claim 49, wherein the one or more catalysts include a transition metal-containing catalyst.

51. The method of claim 49, wherein the one or more catalysts include MeReO₃ and oxides and halides of Co, Mn, Cu, and Re.

52. The method of claim 49, wherein the one or more catalysts include Fe(acac)₂ or Fe(acac)₃

53. The method of claim 49, wherein the catalyst system includes a cocatalyst.

54. The method of claim 53, wherein the cocatalyst includes hydroxylated amines.

55. The method of claim 53, wherein the cocatalyst includes hydroxysuccinamide (NHS) or hydroxylamine.

56. The method of claim 53, wherein the co-catalyst includes N-hydroxyphthalimide (NHPI).

57. The method of claim 54, wherein the co-catalyst includes include NO.

58. A method for making a biocontrol agent comprising:
culturing B. bassiana GHA in a culture medium that includes polypropylene digestion products; and
collecting spores from the B. bassiana GHA therefrom.

59. The method of claim 58 wherein the culture medium is GMM and/or MM are used as controls.
